(19) United States
(12) Patent Application Publication
(10) Pub. No.: US 2026/0308687 A1
(43) Pub. Date: Oct. 8, 2026

(54) COMPOSITION FOR DETECTING LIVER CANCER, AND USE THEREOF

(71) Applicant: BIOCHAIN (BEIJING) SCIENCE & TECHNOLOGY, INC., Beijing (CN)

(72) Inventors: Shuangzhen GU, Beijing (CN); Haizhen NIE, Beijing (CN); Yalin WANG, Beijing (CN); Zhen WU, Beijing (CN); Jinxia GUAN, Beijing (CN); Ming LIAN, Beijing (CN); Xiuxiu WANG, Beijing (CN); Ningning WU, Beijing (CN); Yadong YANG, Beijing (CN); Yongjun LI, Beijing (CN); Huanyue ZHANG, Beijing (CN); Shuanping LIU, Beijing (CN); Qi LI, Beijing (CN); Yuanyuan GUO, Beijing (CN); Chuang WEI, Beijing (CN); Guangpeng ZHOU, Beijing (CN)

(73) Assignee: BIOCHAIN (BEIJING) SCIENCE & TECHNOLOGY, INC., Beijing (CN)

(21) Appl. No.: 19/479,136

(22) PCT Filed: Apr. 16, 2024
(86) PCT No.: PCT/CN2024/087941

§ 371 Date: Oct. 27, 2025

(30) Foreign Application Priority Data
Apr. 26, 2023 (CN) 202310463247.6
Apr. 26, 2023 (CN) 202310463252.7
Apr. 26, 2023 (CN) 202310465237.6
Apr. 26, 2023 (CN) 202310467788.6

(51) Int. Cl. C12Q 1/6886; C12Q 1/6806; C12Q 1/6827; C12Q 1/6851

(52) U.S. Cl. CPC C12Q 1/6886; C12Q 1/6806; C12Q 1/6827; C12Q 1/6851; C12Q 2600/154

(57) ABSTRACT

Provided are a composition for detecting liver cancer and a use thereof. The composition comprises: a nucleic acid used for detecting the methylation status of a target gene, wherein the target gene is one or more of: a CHFR gene, an SPINT2 gene, an RNF135 gene, a CHST2 gene, an SPDYA gene, a PAX5 gene, a VASH2 gene, a TSPYL5 gene, and an EMX1 gene. Further provided are a reagent test kit comprising the composition, and a use of the composition in the preparation of a reagent test kit for the in vitro detection of liver cancer.

### TECHNICAL FIELD

The present application belongs to the field of molecular biology and relates to gene detection, and in particular to a nucleic acid composition for detecting methylation of liver cancer-related genes and a corresponding kit and use thereof.

### BACKGROUND ART

Liver cancer is a highly malignant cancer. An important factor leading to the high mortality rate of liver cancer is the low diagnosis rate of early-stage liver cancer. The cure rate of early-stage liver cancer is much higher than that of middle- and late-stage liver cancer. However, due to the lack of obvious and specific symptoms in early-stage liver cancer, most patients have already developed into the middle and late stages when they are diagnosed. Clinical studies have found that it takes an average of several years a course from the formation of cancer lesions to patients developing clinical symptoms; this provides an effective window period for detecting early-stage liver cancer and improving the diagnosis rate of early-stage liver cancer. Making full use of this window period is expected to improve the treatment effect of liver cancer and reduce the mortality rate of liver cancer. Currently, the technologies used in clinical practice for the diagnosis and screening of early-stage liver cancer are mainly imaging detection technologies, including chest X-rays and low-dose spiral CT (LD-CT). However, it has been found based on large-scale clinical data analysis that chest X-ray examinations have no significant effect on reducing liver cancer mortality; although LD-CT has a significant effect on reducing liver cancer mortality, it has an extremely high false positive rate (90%), so there is a risk of overdiagnosis leading to overtreatment. In addition, the limitations on imaging examinations in terms of equipment cost, operating techniques, and radioactivity make it difficult to promote them on a large scale as a liver cancer screening technology.

Traditional serum tumor markers (such as Cyfra21-SCC, CEA, etc.) have low sensitivity for liver cancer detection, especially for early-stage liver cancer detection, and cannot fully meet the screening requirements for early-stage cancer.

Recent studies have shown that epigenetics plays an important role in the occurrence and development of cancer. As an important mechanism of epigenetics, the regulation of DNA methylation in various cancers has been intensively studied. Research data show that the regulation of gene methylation is related to biological mechanisms such as chromatin structure and gene expression regulation; changes in cellular gene methylation occur in the early stages of tumor formation and throughout the occurrence and development of cancer; methylation of tumor suppressor genes is an important molecular mechanism for the transformation of precancerous lesions into malignant tumor cells. However, there is currently a lack of detection technologies, methods and products for liver cancer methylation gene detection. Therefore, there is a need for methylation gene markers with high sensitivity and high specificity for liver cancer detection.

### SUMMARY OF THE INVENTION

Currently, the technologies used in clinical practice for the diagnosis and screening of early-stage liver cancer are mainly imaging detection technologies, including chest X-rays and low-dose spiral CT (LD-CT). It has been found based on large-scale clinical data analysis that chest X-ray examinations have no significant effect on reducing liver cancer mortality; LD-CT has a significant effect on reducing liver cancer mortality, but has an extremely high false positive rate (90%), so there is a risk of overdiagnosis leading to overtreatment. In addition, the limitations on imaging examinations in terms of equipment cost, operating techniques, and radioactivity make it difficult to promote them on a large scale as a liver cancer screening technology.

However, traditional serum tumor markers (such as Cyfra21-SCC, CEA, etc.) have low sensitivity for liver cancer detection, especially for early-stage liver cancer detection, and cannot fully meet the requirements of early-stage cancer screening.

Based on the problems present in the existing liver cancer detection, the purpose of the present application is to provide a composition for detecting liver cancer in vitro and a kit and use thereof, a method for performing detection based on the kit, and its use for detecting liver cancer.

The specific technical solutions of the present application are as follows:
1. A composition for detecting liver cancer in vitro, comprising:
a nucleic acid used for detecting the methylation status of a target gene,
wherein, the methylation status of the target gene is characterized by the methylation of the target sequence of the target gene,
wherein, the target gene is one or more of: a CHFR gene, an SPINT2 gene, an RNF135 gene, a CHST2 gene, an SPDYA gene, a PAX5 gene, a VASH2 gene, a TSPYL5 gene and an EMX1 gene.
2. The composition according to item 1, wherein the target gene is one or more of: an RNF135 gene, a CHFR gene, an SPINT2 gene, an SPDYA gene, a PAX5 gene and a VASH2 gene.
3. The composition according to item 1, wherein the target gene is one, two or three of: an RNF135 gene, a CHFR gene and a PAX5 gene.
4. The composition according to item 1, wherein the target sequence of the CHFR gene is shown as SEQ ID NO: 1 or SEQ ID NO: 2 or SEQ ID NO: 3 or SEQ ID NO: 4, or comprises the sequence shown as SEQ ID NO: 1 or SEQ ID NO: 2 or SEQ ID NO: 3 or SEQ ID NO: 4.
5. The composition according to item 1, wherein the target sequence of the SPINT2 gene is shown as SEQ ID NO: 5 or SEQ ID NO: 6 or SEQ ID NO: 7 or SEQ ID NO: 8, or comprises the sequence shown as SEQ ID NO: 5 or SEQ ID NO: 6 or SEQ ID NO: 7 or SEQ ID NO: 8.
6. The composition according to item 1, wherein the target sequence of the RNF135 gene is shown as SEQ ID NO: 9 or SEQ ID NO: 10 or SEQ ID NO: 11 or SEQ ID NO: 12, or comprises the sequence shown as SEQ ID NO: 9 or SEQ ID NO: 10 or SEQ ID NO: 11 or SEQ ID NO: 12.
7. The composition according to item 1, wherein the target sequence of the CHST2 gene is shown as SEQ ID NO: 13 or SEQ ID NO: 14 or SEQ ID NO: 15 or SEQ ID NO: 16, or comprises the sequence shown as SEQ ID NO: 13 or SEQ ID NO: 14 or SEQ ID NO: 15 or SEQ ID NO: 16.
8. The composition according to item 1, wherein the target sequence of the SPDYA gene is shown as SEQ ID NO: 17 or SEQ ID NO: 18 or SEQ ID NO: 19 or SEQ ID NO: 20, or comprises the sequence shown as SEQ ID NO: 17 or SEQ ID NO: 18 or SEQ ID NO: 19 or SEQ ID NO: 20.
9. The composition according to item 1, wherein the target sequence of the PAX5 gene is shown as SEQ ID NO: 21 or SEQ ID NO: 22 or SEQ ID NO: 23 or SEQ ID NO: 24, or comprises the sequence shown as SEQ ID NO: 21 or SEQ ID NO: 22 or SEQ ID NO: 23 or SEQ ID NO: 24.
10. The composition according to item 1, wherein the target sequence of the VASH2 gene is shown as SEQ ID NO: 25 or SEQ ID NO: 26 or SEQ ID NO: 27 or SEQ ID NO: 28, or comprises the sequence shown as SEQ ID NO: 25 or SEQ ID NO: 26 or SEQ ID NO: 27 or SEQ ID NO: 28.
11. The composition according to item 1, wherein the target sequence of the TSPYL5 gene is shown as SEQ ID NO: 29 or SEQ ID NO: 30 or SEQ ID NO: 31 or SEQ ID NO: 32, or comprises the sequence shown as SEQ ID NO: 29 or SEQ ID NO: 30 or SEQ ID NO: 31 or SEQ ID NO: 32.
12. The composition according to item 1, wherein the target sequence of the EMX1 gene is shown as SEQ ID NO: 33 or SEQ ID NO: 34 or SEQ ID NO: 35 or SEQ ID NO: 36, or comprises the sequence shown as SEQ ID NO: 33 or SEQ ID NO: 34 or SEQ ID NO: 35 or SEQ ID NO: 36.
13. The composition according to any one of items 1 to 12, wherein the nucleic acid for detecting the methylation status of the target gene comprises:
a primer, wherein the primer is a fragment of at least 9 nucleotides in the target sequence of the target gene, the fragment comprises at least one CpG dinucleotide sequence.
14. The composition according to any one of items 1 to 13, wherein the nucleic acid for detecting the methylation status of the target gene comprises:
a probe, wherein the probe is a fragment that hybridizes to at least 15 nucleotides of the target sequence of the target gene under moderately stringent or stringent conditions, the fragment comprises at least one CpG dinucleotide sequence.
15. The composition according to any one of items 1 to 14, further comprising:
a reagent that converts 5-unmethylated cytosine base of the target sequence of the target gene to uracil.
16. The composition according to any one of items 1 to 15, wherein the nucleic acid for detecting the methylation status of the target gene further comprises:
a blocker that preferentially bind to the target sequence in its unmethylated state.
17. The composition according to item 16, wherein the fragment of at least 9 nucleotides is a sequence of SEQ ID NO:37 and SEQ ID NO:38, or a sequence of SEQ ID NO:40 and SEQ ID NO:41, or a sequence of SEQ ID NO:43 and SEQ ID NO: 44, or a sequence of SEQ ID NO:46 and SEQ ID NO:47, or a sequence of SEQ ID NO:49 and SEQ ID NO:50, or a sequence of SEQ ID NO:52 and SEQ ID NO:53, a sequence of SEQ ID NO:55 and SEQ ID NO:56, or a sequence of SEQ ID NO:58 and SEQ ID NO:59, or a sequence of SEQ ID NO: 61 and SEQ ID NO:62;
the fragment of at least 15 nucleotides is a sequence of SEQ ID NO:39, or a sequence of SEQ ID NO: 42, or a sequence of SEQ ID NO:45, or a sequence of SEQ ID NO:48, or a sequence of SEQ ID NO: 51, or a sequence of SEQ ID NO:54, or a sequence of SEQ ID NO:57, or a sequence of SEQ ID NO: 60, or a sequence of SEQ ID NO:63.
18. An oligonucleotide for detecting liver cancer in vitro, comprising:
a fragment of at least 9 nucleotides of SEQ ID NO: 1 or SEQ ID NO: 2 or SEQ ID NO: 3 or SEQ ID NO: 4 or a complementary sequence thereof and comprising at least one CpG dinucleotide sequence; and/or
a fragment of at least 9 nucleotides of SEQ ID NO: 5 or SEQ ID NO: 6 or SEQ ID NO: 7 or SEQ ID NO: 8 or a complementary sequence thereof and comprising at least one CpG dinucleotide sequence; and/or
a fragment of at least 9 nucleotides of SEQ ID NO: 9 or SEQ ID NO: 10 or SEQ ID NO: 11 or SEQ ID NO: 12 or a complementary sequence thereof and comprising at least one CpG dinucleotide sequence; and/or
a fragment of at least 9 nucleotides of SEQ ID NO: 13 or SEQ ID NO: 14 or SEQ ID NO: 15 or SEQ ID NO: 16 or a complementary sequence thereof and comprising at least one CpG dinucleotide sequence; and/or
a fragment of at least 9 nucleotides of SEQ ID NO: 17 or SEQ ID NO: 18 or SEQ ID NO: 19 or SEQ ID NO: 20 or a complementary sequence thereof and comprising at least one CpG dinucleotide sequence; and/or
a fragment of at least 9 nucleotides of SEQ ID NO: 21 or SEQ ID NO: 22 or SEQ ID NO: 23 or SEQ ID NO: 24 or a complementary sequence thereof and comprising at least one CpG dinucleotide sequence; and/or
a fragment of at least 9 nucleotides of SEQ ID NO: 25 or SEQ ID NO: 26 or SEQ ID NO: 27 or SEQ ID NO: 28 or a complementary sequence thereof and comprising at least one CpG dinucleotide sequence; and/or
a fragment of at least 9 nucleotides of SEQ ID NO: 29 or SEQ ID NO: 30 or SEQ ID NO: 31 or SEQ ID NO: 32 or a complementary sequence thereof and comprising at least one CpG dinucleotide sequence; and/or
a fragment of at least 9 nucleotides of SEQ ID NO: 33 or SEQ ID NO: 34 or SEQ ID NO: 35 or SEQ ID NO: 36 or a complementary sequence thereof and comprising at least one CpG dinucleotide sequence.
19. The oligonucleotide according to item 18, further comprising:
a fragment that hybridizes to at least 15 nucleotides of SEQ ID NO: 1 or SEQ ID NO: 2 or SEQ ID NO: 3 or SEQ ID NO: 4 or a complementary sequence thereof under moderately stringent or stringent conditions and comprises at least one CpG dinucleotide sequence; and/or
a fragment that hybridizes to at least 15 nucleotides of SEQ ID NO: 5 or SEQ ID NO: 6 or SEQ ID NO: 7 or SEQ ID NO: 8 or a complementary sequence thereof under moderately stringent or stringent conditions and comprises at least one CpG dinucleotide sequence; and/or
a fragment that hybridizes to at least 15 nucleotides of SEQ ID NO: 9 or SEQ ID NO: 10 or SEQ ID NO: 11 or SEQ ID NO: 12 or a complementary sequence thereof under moderately stringent or stringent conditions and comprises at least one CpG dinucleotide sequence; and/or
a fragment that hybridizes to at least 15 nucleotides of SEQ ID NO: 13 or SEQ ID NO: 14 or SEQ ID NO: 15 or SEQ ID NO: 16 or a complementary sequence thereof under moderately stringent or stringent conditions and comprises at least one CpG dinucleotide sequence; and/or
a fragment that hybridizes to at least 15 nucleotides of SEQ ID NO: 17 or SEQ ID NO: 18 or SEQ ID NO: 19 or SEQ ID NO: 20 or a complementary sequence thereof under moderately stringent or stringent conditions and comprises at least one CpG dinucleotide sequence; and/or
a fragment that hybridizes to at least 15 nucleotides of SEQ ID NO: 21 or SEQ ID NO: 22 or SEQ ID NO: 23 or SEQ ID NO: 24 or a complementary sequence thereof under moderately stringent or stringent conditions and comprises at least one CpG dinucleotide sequence; and/or
a fragment that hybridizes to at least 15 nucleotides of SEQ ID NO: 25 or SEQ ID NO: 26 or SEQ ID NO: 27 or SEQ ID NO: 28 or a complementary sequence thereof under moderately stringent or stringent conditions and comprises at least one CpG dinucleotide sequence; and/or
a fragment that hybridizes to at least 15 nucleotides of SEQ ID NO: 29 or SEQ ID NO: 30 or SEQ ID NO: 31 or SEQ ID NO: 32 or a complementary sequence thereof under moderately stringent or stringent conditions and comprises at least one CpG dinucleotide sequence; and/or
a fragment that hybridizes to at least 15 nucleotides of SEQ ID NO: 33, SEQ ID NO: 34, SEQ ID NO: 35, or SEQ ID NO: 36 or a complementary sequence thereof under moderately stringent or stringent conditions and comprises at least one CpG dinucleotide sequence.
20. The oligonucleotide according to item 19, further comprising:
a blocker that preferentially bind to the target sequence in its unmethylated state.
21. An oligonucleotide for detecting liver cancer in vitro, comprising:
the sequences of SEQ ID NO: 37 and SEQ ID NO: 38; or the sequences of SEQ ID NO: 40 and SEQ ID NO: 41; or the sequences of SEQ ID NO: 43 and SEQ ID NO: 44; or the sequences of SEQ ID NO: 46 and SEQ ID NO: 47; or the sequences of SEQ ID NO: 49 and SEQ ID NO: 50; or the sequences of SEQ ID NO: 52 and SEQ ID NO: 53; or the sequences of SEQ ID NO: 55 and SEQ ID NO: 56; or the sequences of SEQ ID NO: 58 and SEQ ID NO: 59; or the sequences of SEQ ID NO: 61 and SEQ ID NO: 62.
22. The oligonucleotide according to item 21, further comprising:
the sequence of SEQ ID NO: 39; or the sequence of SEQ ID NO: 42; or the sequence of SEQ ID NO: 45; or the sequence of SEQ ID NO: 48; or the sequence of SEQ ID NO: 51; or the sequence of SEQ ID NO: 54; or the sequence of SEQ ID NO: 57; or the sequence of SEQ ID NO: 60; or the sequence of SEQ ID NO: 63.
23. Use of the composition of any one of items 1 to 17 or the oligonucleotide of any one of items 18 to 22 in detecting liver cancer in vitro.
24. Use of the composition of any one of items 1 to 17 or the oligonucleotide of any one of items 18 to 22 in the preparation of a kit for detecting liver cancer in vitro.
25. A kit comprising the composition of any one of items 1 to 17 or the oligonucleotide of any one of items 18 to 22.
26. The kit according to item 25, further comprising at least one other component selected from the group consisting of:
a nucleoside triphosphate, a DNA polymerase, and a buffer required for the function of the DNA polymerase.
27. The kit according to item 25 or 26, wherein the samples detected by the kit comprises: cell lines, histological sections, tissue biopsies/paraffin-embedded tissues, body fluid, feces, colon effluent, urine, plasma, serum, whole blood, isolated blood cells, cells isolated from blood, or a combination thereof.
28. A method for detecting liver cancer, comprising the following steps:
isolating a DNA sample comprising a target sequence of a target gene or a fragment thereof from a biological sample to be tested;
determining the methylation status of the target sequence of the target gene; and
determining the state of the biological sample based on the detection result of the methylation status of the target sequence of the target gene, thereby realizing in vitro detection of liver cancer.
29. A method for detecting liver cancer, comprising the following steps:
extracting genomic DNA from the biological sample to be tested;
treating the extracted genomic DNA with a reagent to convert the 5-unmethylated cytosine base to uracil or other bases;
contacting the DNA sample treated with the reagent with a DNA polymerase and a primer of a target sequence of a target gene to initiate a DNA polymerization reaction;
detecting the amplified product with a probe; and
determining the methylation status of at least one CpG dinucleotide of the target sequence of the target gene based on whether the amplified product is present or not.
30. The method according to item 28 or 29, wherein the target gene is one or more of: a CHFR gene, an SPINT2 gene, an RNF135 gene, a CHST2 gene, an SPDYA gene, a PAX5 gene, a VASH2 gene, a TSPYL5 gene and an EMX1 gene.
31. The method according to item 28 or 29, wherein the target gene is one or more of: an RNF135 gene, a CHFR gene, an SPINT2 gene, an SPDYA gene, a PAX5 gene and a VASH2 gene.
32. The method according to item 28 or 29, wherein the target gene is one, two or three of: an RNF135 gene, a CHFR gene and a PAX5 gene.
33. The method according to any one of items 30 to 32, wherein the target sequence of the CHFR gene is shown as SEQ ID NO: 1 or SEQ ID NO: 2 or SEQ ID NO: 3 or SEQ ID NO: 4, or comprises the sequence shown as SEQ ID NO: 1 or SEQ ID NO: 2 or SEQ ID NO: 3 or SEQ ID NO: 4.
34. The method according to any one of items 30 to 32, wherein the target sequence of the SPINT2 gene is shown as SEQ ID NO: 5 or SEQ ID NO: 6 or SEQ ID NO: 7 or SEQ ID NO: 8, or comprises the sequence shown as SEQ ID NO: 5 or SEQ ID NO: 6 or SEQ ID NO: 7 or SEQ ID NO: 8.
35. The method according to any one of items 30 to 32, wherein the target sequence of the RNF135 gene is shown as SEQ ID NO: 9 or SEQ ID NO: 10 or SEQ ID NO: 11 or SEQ ID NO: 12, or comprises the sequence shown as SEQ ID NO: 9 or SEQ ID NO: 10 or SEQ ID NO: 11 or SEQ ID NO: 12.
36. The method according to any one of items 30 to 32, wherein the target sequence of the CHST2 gene is shown as SEQ ID NO: 13 or SEQ ID NO: 14 or SEQ ID NO: 15 or SEQ ID NO: 16, or comprises the sequence shown as SEQ ID NO: 13 or SEQ ID NO: 14 or SEQ ID NO: 15 or SEQ ID NO: 16.
37. The method according to any one of items 30 to 32, wherein the target sequence of the SPDYA gene is shown as SEQ ID NO: 17 or SEQ ID NO: 18 or SEQ ID NO: 19 or SEQ ID NO: 20, or comprises the sequence shown as SEQ ID NO: 17 or SEQ ID NO: 18 or SEQ ID NO: 19 or SEQ ID NO: 20.
38. The method according to any one of items 30 to 32, wherein the target sequence of the PAX5 gene is shown as SEQ ID NO: 21 or SEQ ID NO: 22 or SEQ ID NO: 23 or SEQ ID NO: 24, or comprises the sequence shown as SEQ ID NO: 21 or SEQ ID NO: 22 or SEQ ID NO: 23 or SEQ ID NO: 24.
39. The method according to any one of items 30 to 32, wherein the target sequence of the VASH2 gene is shown as SEQ ID NO: 25 or SEQ ID NO: 26 or SEQ ID NO: 27 or SEQ ID NO: 28, or comprises the sequence shown as SEQ ID NO: 25 or SEQ ID NO: 26 or SEQ ID NO: 27 or SEQ ID NO: 28.
40. The method according to any one of items 30 to 32, wherein the target sequence of the TSPYL5 gene is shown as SEQ ID NO: 29 or SEQ ID NO: 30 or SEQ ID NO: 31 or SEQ ID NO: 32, or comprises the sequence shown as SEQ ID NO: 29 or SEQ ID NO: 30 or SEQ ID NO: 31 or SEQ ID NO: 32.
41. The method according to any one of items 30 to 32, wherein the target sequence of the EMX1 gene is shown as SEQ ID NO: 33 or SEQ ID NO: 34 or SEQ ID NO: 35 or SEQ ID NO: 36, or comprises the sequence shown as SEQ ID NO: 33 or SEQ ID NO: 34 or SEQ ID NO: 35 or SEQ ID NO: 36.
42. The method according to item 29, wherein the reagent is a bisulfite reagent.
43. The method according to item 29, wherein the primer is:
a fragment of at least 9 nucleotides of SEQ ID NO: 1 or SEQ ID NO: 2 or SEQ ID NO: 3 or SEQ ID NO: 4 or a complementary sequence thereof and comprising at least one CpG dinucleotide sequence; and/or
a fragment of at least 9 nucleotides of SEQ ID NO: 5 or SEQ ID NO: 6 or SEQ ID NO: 7 or SEQ ID NO: 8 or a complementary sequence thereof and comprising at least one CpG dinucleotide sequence; and/or
a fragment of at least 9 nucleotides of SEQ ID NO: 9 or SEQ ID NO: 10 or SEQ ID NO: 11 or SEQ ID NO: 12 or a complementary sequence thereof and comprising at least one CpG dinucleotide sequence; and/or
a fragment of at least 9 nucleotides of SEQ ID NO: 13 or SEQ ID NO: 14 or SEQ ID NO: 15 or SEQ ID NO: 16 or a complementary sequence thereof and comprising at least one CpG dinucleotide sequence; and/or
a fragment of at least 9 nucleotides of SEQ ID NO: 17 or SEQ ID NO: 18 or SEQ ID NO: 19 or SEQ ID NO: 20 or a complementary sequence thereof and comprising at least one CpG dinucleotide sequence; and/or
a fragment of at least 9 nucleotides of SEQ ID NO: 21 or SEQ ID NO: 22 or SEQ ID NO: 23 or SEQ ID NO: 24 or a complementary sequence thereof and comprising at least one CpG dinucleotide sequence; and/or
a fragment of at least 9 nucleotides of SEQ ID NO: 25 or SEQ ID NO: 26 or SEQ ID NO: 27 or SEQ ID NO: 28 or a complementary sequence thereof and comprising at least one CpG dinucleotide sequence; and/or
a fragment of at least 9 nucleotides of SEQ ID NO: 29 or SEQ ID NO: 30 or SEQ ID NO: 31 or SEQ ID NO: 32 or a complementary sequence thereof and comprising at least one CpG dinucleotide sequence; and/or
a fragment of at least 9 nucleotides of SEQ ID NO: 33 or SEQ ID NO: 34 or SEQ ID NO: 35 or SEQ ID NO: 36 or a complementary sequence thereof and comprising at least one CpG dinucleotide sequence.
44. The method according to item 29, wherein the probe is:
a fragment that hybridizes to at least 15 nucleotides of SEQ ID NO: 1 or SEQ ID NO: 2 or SEQ ID NO: 3 or SEQ ID NO: 4 or a complementary sequence thereof under moderately stringent or stringent conditions and comprises at least one CpG dinucleotide sequence; and/or
a fragment that hybridizes to at least 15 nucleotides of SEQ ID NO: 5 or SEQ ID NO: 6 or SEQ ID NO: 7 or SEQ ID NO: 8 or a complementary sequence thereof under moderately stringent or stringent conditions and comprises at least one CpG dinucleotide sequence; and/or
a fragment that hybridizes to at least 15 nucleotides of SEQ ID NO: 9 or SEQ ID NO: 10 or SEQ ID NO: 11 or SEQ ID NO: 12 or a complementary sequence thereof under moderately stringent or stringent conditions and comprises at least one CpG dinucleotide sequence; and/or
a fragment that hybridizes to at least 15 nucleotides of SEQ ID NO: 13 or SEQ ID NO: 14 or SEQ ID NO: 15 or SEQ ID NO: 16 or a complementary sequence thereof under moderately stringent or stringent conditions and comprises at least one CpG dinucleotide sequence; and/or
a fragment that hybridizes to at least 15 nucleotides of SEQ ID NO: 17 or SEQ ID NO: 18 or SEQ ID NO: 19 or SEQ ID NO: 20 or a complementary sequence thereof under moderately stringent or stringent conditions and comprises at least one CpG dinucleotide sequence; and/or
a fragment that hybridizes to at least 15 nucleotides of SEQ ID NO: 21 or SEQ ID NO: 22 or SEQ ID NO: 23 or SEQ ID NO: 24 or a complementary sequence thereof under moderately stringent or stringent conditions and comprises at least one CpG dinucleotide sequence; and/or
a fragment that hybridizes to at least 15 nucleotides of SEQ ID NO: 25 or SEQ ID NO: 26 or SEQ ID NO: 27 or SEQ ID NO: 28 or a complementary sequence thereof under moderately stringent or stringent conditions and comprises at least one CpG dinucleotide sequence; and/or
a fragment that hybridizes to at least 15 nucleotides of SEQ ID NO: 29 or SEQ ID NO: 30 or SEQ ID NO: 31 or SEQ ID NO: 32 or a complementary sequence thereof under moderately stringent or stringent conditions and comprises at least one CpG dinucleotide sequence; and/or
a fragment that hybridizes to at least 15 nucleotides of SEQ ID NO: 33, SEQ ID NO: 34, SEQ ID NO: 35, or SEQ ID NO: 36 or a complementary sequence thereof under moderately stringent or stringent conditions and comprises at least one CpG dinucleotide sequence.
45. The method according to item 43, wherein the primers are of sequences of SEQ ID NO: 37 and SEQ ID NO: 38, or sequences of SEQ ID NO: 40 and SEQ ID NO: 41, or sequences of SEQ ID NO: 43 and SEQ ID NO: 44, or sequences of SEQ ID NO: 46 and SEQ ID NO: 47, or sequences of SEQ ID NO: 49 and SEQ ID NO: 50, or sequences of SEQ ID NO: 52 and SEQ ID NO: 53, sequences of SEQ ID NO: 55 and SEQ ID NO: 56, or sequences of SEQ ID NO: 58 and SEQ ID NO: 59, or sequences of SEQ ID NO: 61 and SEQ ID NO: 62.
46. The method according to item 44, wherein the probe is a sequence of SEQ ID NO: 39, or a sequence of SEQ ID NO: 42, or a sequence of SEQ ID NO: 45, or a sequence of SEQ ID NO: 48, or a sequence of SEQ ID NO: 51, or a sequence of SEQ ID NO: 54, or a sequence of SEQ ID NO: 57, or a sequence of SEQ ID NO: 60, or a sequence of SEQ ID NO: 63.
47. A system for detecting liver cancer, wherein the system comprises:
a data acquisition module, for acquiring data on the methylation marker levels in a subject sample; and
a calculation module of the probability of suffering from liver cancer, for calculating the data obtained in the data acquisition module, thereby calculating the probability of the subject suffering from liver cancer,
wherein the methylation marker(s) is/are one, two or three of an RNF135 gene, a CHFR gene and a PAX5 gene.
48. The system according to item 47, wherein the target sequence of the RNF135 gene is shown as SEQ ID NO: 1 or SEQ ID NO: 2 or SEQ ID NO: 3 or SEQ ID NO: 4, or comprises the sequence shown as SEQ ID NO: 1 or SEQ ID NO: 2 or SEQ ID NO: 3 or SEQ ID NO: 4.
49. The system according to item 47, wherein the target sequence of the CHFR gene is shown as SEQ ID NO: 5 or SEQ ID NO: 6 or SEQ ID NO: 7 or SEQ ID NO: 8, or comprises the sequence shown as SEQ ID NO: 5 or SEQ ID NO: 6 or SEQ ID NO: 7 or SEQ ID NO: 8.
50. The system according to item 47, wherein the target sequence of the PAX5 gene is shown as SEQ ID NO:9 or SEQ ID NO:10 or SEQ ID NO:11 or SEQ ID NO:12, or comprises the sequence shown as SEQ ID NO:9 or SEQ ID NO: 10 or SEQ ID NO: 11 or SEQ ID NO:12.
51. The system according to any one of items 47 to 50, wherein the data acquisition module comprises the following submodules:
an extraction module, for extracting genomic DNA from a sample;
a reagent treatment module, for treating the extracted genomic DNA with a bisulfite reagent to convert the 5-unmethylated cytosine base to uracil or other bases;
a detection module, for detecting the genomic DNA treated with a bisulfite reagent to obtain the methylation marker level of the sample.
52. The system according to item 51, wherein the genomic DNA treated with a bisulfite reagent is detected using real-time fluorescent quantitative PCR.
53. The system according to item 52, wherein the primers used are of sequences of SEQ ID NO: 13 and SEQ ID NO: 14, or sequences of SEQ ID NO: 16 and SEQ ID NO: 17, or sequences of SEQ ID NO: 19 and SEQ ID NO: 20.
54. The system according to item 52, wherein the probe used is of the sequence of SEQ ID NO: 15, or the sequence of SEQ ID NO: 18, or the sequence of SEQ ID NO: 21.
55. The system according to any one of items 47 to 54, wherein in the calculation module for the probability of suffering from liver cancer, a pre-constructed formula for calculating the probability of a subject suffering from liver cancer based on data fitting of methylation marker levels is pre-stored.
56. The system according to item 55, wherein the formula is obtained by fitting the data of the methylation level of RNF135 in an existing database.
57. The system according to item 55 or 56, wherein the formula is obtained by fitting the data of the methylation level of RNF135 and the methylation level of PAX5 in an existing database.
58. The system according to any one of items 55-57, wherein the formula is obtained by fitting the data of the methylation level of RNF135, the methylation level of PAX5, and the methylation level of CHFR in an existing database.
59. The system according to any one of items 55-58, wherein the formula is obtained by fitting the data of the methylation level of RNF135, the methylation level of PAX5, and the methylation level of CHFR in the existing database using generalized linear regression.
60. The system according to any one of items 55-59, wherein the formula is:

$$P = {e}^{( a * M 1 + b * M 2 + c * M 3 + 81 )} / ( {e}^{( a * M 1 + b * M 2 + c * M 3 + 81 )} + 1 ) ,$$

wherein P represents the probability of the subject suffering from liver cancer, M1 represents the Ct value of RNF135 determined by real-time fluorescence quantitative PCR, M2 represents the Ct value of PAX5 determined by real-time fluorescence quantitative PCR, and M3 represents the Ct value of CHFR determined by real-time fluorescence quantitative PCR,
a is selected from any value between 0.8 and 1.2, preferably 0.9;
b is selected from any value between 0.6 and 0.9, preferably 0.7;
c is selected from any value between 0.1 and 0.5, preferably 0.3.
61. The system according to any one of items 47 to 60, wherein when the probability of the subject suffering from liver cancer is greater than or equal to 0.24, the subject is determined to have liver cancer.
62. The system of any one of items 47-61, wherein the sample comprises: cell lines, histological sections, tissue biopsies/paraffin-embedded tissues, body fluid, feces, colon effluents, urine, plasma, serum, whole blood, isolated blood cells, cells isolated from blood, or a combination thereof.
63. A method for detecting liver cancer, comprising:
a data acquisition step, for acquiring data on the methylation marker levels in a subject sample; and
a step of calculating the probability of suffering from liver cancer, for calculating the data obtained in the data acquisition step, thereby calculating the probability of the subject suffering from liver cancer,
wherein the methylation marker(s) is/are one, two or three of an RNF135 gene, a CHFR gene and a PAX5 gene.
64. The method according to item 63, wherein the target sequence of the RNF135 gene is shown as SEQ ID NO: 1 or SEQ ID NO: 2 or SEQ ID NO: 3 or SEQ ID NO: 4, or comprises the sequence shown as SEQ ID NO: 1 or SEQ ID NO: 2 or SEQ ID NO: 3 or SEQ ID NO: 4.
65. The method according to item 63, wherein the target sequence of the CHFR gene is shown as SEQ ID NO: 5 or SEQ ID NO: 6 or SEQ ID NO: 7 or SEQ ID NO: 8, or comprises the sequence shown as SEQ ID NO: 5 or SEQ ID NO: 6 or SEQ ID NO: 7 or SEQ ID NO: 8.
66. The method according to item 63, wherein the target sequence of the PAX5 gene is shown as SEQ ID NO: 9 or SEQ ID NO: 10 or SEQ ID NO: 11 or SEQ ID NO: 12, or comprises the sequence shown as SEQ ID NO: 9 or SEQ ID NO: 10 or SEQ ID NO: 11 or SEQ ID NO: 12.
67. The method according to any one of items 63 to 66, wherein the data acquisition step comprises the following sub-steps:
an extraction step, for extracting genomic DNA from a sample;
a reagent treatment step, for treating the extracted genomic DNA with a bisulfite reagent to convert the 5-unmethylated cytosine base to uracil or other bases;
a detection step, for detecting the genomic DNA treated with a bisulfite reagent to obtain the methylation marker level of the sample.
68. The method according to item 67, wherein the genomic DNA treated with a bisulfite reagent is detected using real-time fluorescent quantitative PCR.
69. The method according to item 68, wherein the primers used are of sequences of SEQ ID NO: 13 and SEQ ID NO: 14, or sequences of SEQ ID NO: 16 and SEQ ID NO: 17, or sequences of SEQ ID NO: 19 and SEQ ID NO: 20.
70. The method according to item 68, wherein the probe used is of the sequence of SEQ ID NO: 15, or the sequence of SEQ ID NO: 18, or the sequence of SEQ ID NO: 21.
71. The method according to any one of items 63 to 70, wherein in the step of calculating the probability of suffering from liver cancer, a pre-constructed formula for calculating the probability of a subject suffering from liver cancer based on data fitting of methylation marker levels is pre-stored.
72. The method according to item 71, wherein the formula is obtained by fitting the data of the methylation level of RNF135 in an existing database.
73. The method according to item 71 or 72, wherein the formula is obtained by fitting the data of the methylation level of RNF135 and the methylation level of PAX5 in an existing database.
74. The method according to any one of items 71-73, wherein the formula is obtained by fitting the data of the methylation level of RNF135, the methylation level of PAX5, and the methylation level of CHFR in an existing database.
75. The method according to any one of items 71-74, wherein the formula is obtained by fitting the data of the methylation level of RNF135, the methylation level of PAX5, and the methylation level of CHFR in the existing database using generalized linear regression.
76. The method according to any one of items 71-75, wherein the formula is:

$$P = {e}^{( a * M 1 + b * M 2 + c * M 3 + 81 )} / ( {e}^{( a * M 1 + b * M 2 + c * M 3 + 81 )} + 1 ) ,$$

wherein P represents the probability of the subject suffering from liver cancer, M1 represents the Ct value of RNF135 determined by real-time fluorescence quantitative PCR, M2 represents the Ct value of PAX5 determined by real-time fluorescence quantitative PCR, and M3 represents the Ct value of CHFR determined by real-time fluorescence quantitative PCR,
a is selected from any value between 0.8 and 1.2, preferably 0.9;
b is selected from any value between 0.6 and 0.9, preferably 0.7;
c is selected from any value between 0.1 and 0.5, preferably 0.3.
77. The method according to any one of items 63 to 76, wherein when the probability of the subject suffering from liver cancer is greater than or equal to 0.24, the subject is determined to have liver cancer.
78. The method of any one of items 63-77, wherein the sample comprises: cell lines, histological sections, tissue biopsies/paraffin-embedded tissues, body fluid, feces, colon effluents, urine, plasma, serum, whole blood, isolated blood cells, cells isolated from blood, or a combination thereof.

The present application has the following beneficial effects:

The present application screened out 9 liver cancer-related markers that can sensitively and specifically detect liver cancer, and identified the methylation regions of the related markers. By detecting the target sequence of the methylation gene of CHFR gene, SPINT2 gene, RNF135 gene, CHST2 gene, SPDYA gene, PAX5 gene, VASH2 gene, TSPYL5 gene or EMX1 gene, the methylation status of the gene can be sensitively and specifically detected, thereby allowing them to be used in the detection of free DNA in peripheral blood. The detection of peripheral blood samples from liver cancer patients and normal control individuals showed that the composition and detection method described herein can sensitively and specifically detect liver cancer, thereby ensuring the accuracy and reliability of the detection results. Therefore, the present application provides a composition, kit and detection method that can be used for in vitro detection of liver cancer, which can detect liver cancer conveniently, quickly and effectively and has important clinical application value.

The present application uses epigenomic and bioinformatics technologies to analyze the methylation data of liver cancer genome, finds multiple methylation genes related to liver cancer, and determines the target sequence of liver cancer methylation genes where methylation abnormalities occur. Through this target sequence of the methylation gene, the methylation status of the gene can be sensitively and specifically detected, which can be used for the detection of free DNA in peripheral blood.

The composition described herein is used to screen asymptomatic people in a non-invasive manner, reduces the harm caused by invasive testing. The described composition has higher sensitivity and accuracy and can achieve real-time monitoring.

### DETAIL DESCRIPTION OF THE INVENTION

The following is a detailed description of the present application. Although specific embodiments of the present application have been shown, it should be understood that the present application can be implemented in various forms and should not be limited to the embodiments set forth herein. On the contrary, these embodiments are provided to enable a more thorough understanding of the present application and to fully convey the scope of the present application to those skilled in the art. Unless otherwise indicated, the implementation of the present application will employ conventional techniques of molecular biology (including recombinant techniques), microbiology, cell biology, biochemistry and genetics, which are all within the scope of conventional technical means in the art. Such techniques are described in detail in the literature, such as Molecular Cloning: A Laboratory Manual, 2nd edition (Sambrook et al., 1989); Oligonucleotide Synthesis (M. J. Gait, 1984 edition); Animal Cell Culture (R. I. Freshney, 1987 edition); Methods in Enzymology series (Academic Press, Inc.); Current Protocols in Molecular Biology (F. M. Ausubel et al., 1987 edition, and periodic updates); PCR: The Polymerase Chain Reaction (Mullis et al., 1994 edition). The primers, probes, blockers and kits used herein can be prepared using standard techniques known in the art. Unless otherwise defined, technical and scientific terms used herein have the same meanings as those commonly understood by ordinary skilled in the art to which the present application belongs.

### Definition

As used herein, “precancerous cells” refer to cells that are in the early stages of transforming into cancer cells or that are prone to transforming into cancer cells. Such cells may exhibit one or more phenotypic traits of characteristic of cancer cells.

As used herein, “stringent hybridization conditions” and “high stringency hybridization conditions” refer to conditions under which a probe will hybridize to its target subsequence, typically in a complex mixture of nucleic acids. Stringent conditions are sequence-dependent and will be different in different circumstances. Longer sequences hybridize specifically at higher temperatures. For detailed guidance on nucleic acid hybridization, see Tijssen, “Overview of principles of hybridization and the strategy of nucleic acid probe assays” in Laboratory Techniquesin Biochemistry and Molecular Biology-Hybridization with Nucleic Acid Probes. Generally, stringent conditions are about 5-10° C. lower than the melting point (Tm) for the specific nucleic acid at a defined ionic strength pH. At the Tm temperature (under defined ionic strength, pH, and nucleic acid concentration), 50% of the probes complementary to the target hybridize uniformly to the target sequence. Stringent conditions may also be achieved by the addition of destabilizing agents. For selective or specific hybridization, a positive signal is twice, preferably 10 times, that of background hybridization. Exemplary stringent hybridization conditions are as follows: hybridization in a solution of 50% formamide, 5×SSC and 1% SDS at 42° C., or hybridization in a solution of 5×SSC and 1% SDS at 65° C., followed by washing in a solution of 0.2×SSC and 0.1% SDS at 65° C.

Furthermore, nucleic acids that do not hybridize under stringent conditions are still substantially similar if the polypeptides that they encode are substantially similar. In such cases, the nucleic acids are typically hybridized under moderately stringent hybridization conditions. Illustratively, “moderately stringent hybridization conditions” include hybridizing in a solution of 40% formamide, 1 M sodium chloride, and 1% SDS at 37° C., and washing in a solution of 1×SSC at 45° C. Guidance on achieving conditions that achieve the same stringency in prior art will be readily apparent to those skilled in the art. For PCR, a temperature of around 36° C. is typically suitable for low stringency amplification, while the annealing temperature ranges from 32° C. to 48° C. based on the length of the primers. For high stringency PCR amplification, it is generally at 62° C., while the annealing temperature for high stringency hybridization ranges from 50° C. to 65° C. based on the length and specificity of the primers. Cycling conditions for high stringency and low stringency amplifications typically include a denaturation phase at 90-95° C. for 30 seconds to 2 minutes, an annealing phase for 30 seconds to 2 minutes, and an extension phase at approximately 72° C. for 1 to 2 minutes. Tools and guidance for low and high stringency amplification reactions are available in the art.

As used herein, “oligonucleotide” refers to a molecule composed of two or more nucleotides, preferably a molecule composed of three or more nucleotides, the precise size of which may depend on many factors, which in turn are determined by the ultimate function and use of the oligonucleotide. In certain specific embodiments, an oligonucleotide can comprise a length of 10 nucleotides to 100 nucleotides. In certain specific embodiments, the oligonucleotide may comprise a length of 10 nucleotides to 30 nucleotides, or may have a length of 20 and 25 nucleotides. In some specific embodiments, oligonucleotides shorter than these lengths may also be suitable.

As used herein, a “primer” refers to an oligonucleotide, whether naturally occurring in a purified restriction digest or produced synthetically, that is capable of serving as a point of initiation of synthesis when being placed under conditions that induce the synthesis of a primer extension product complementary to a nucleic acid chain, i.e., in the presence of nucleotides and an inducing agent such as a DNA or RNA polymerase and at a suitable temperature and pH. The primer may be single-stranded or double-stranded and must be long enough to initiate the synthesis of the desired extension product in the presence of the inducing agent. The exact length of the primers depends on a variety of factors, including temperature, primer source, and the method used. For example, for diagnostic and prognostic applications, oligonucleotide primers typically contain at least or more than about 9, 10, or 15, or 20, or 25 or more nucleotides, but can contain fewer or more nucleotides, depending on the complexity of the target sequence. Factors involved in determining the appropriate length of a primer are well known to those skilled in the art.

As used herein, a “primer pair” refers to a primer pair that hybridizes to opposite strands of a target DNA molecule or to a region of the target DNA that flanks the nucleotide sequence to be amplified.

As used herein, a “primer site” refers to a region of a target DNA or other nucleic acid to which a primer hybridizes.

As used herein, “probe” when referring to a nucleic acid sequence is used in its ordinary sense to refer to a selected nucleic acid sequence that can hybridize to a target sequence under specified conditions and can be used to detect the presence of the target sequence. It will be understood by those skilled in the art that, in certain circumstances, probes can also serve as primers, and primers can serve as probes.

As used herein, “DNA methylation” refers to the addition of a methyl group to the position 5 of cytosine (C), typically (but not necessarily) in the case of a CpG (cytosine followed by guanine) dinucleotide. As used herein, “increased methylation level” or “significant methylation level” refers to the presence of at least one methylated cytosine nucleotide in a DNA sequence, wherein the corresponding C is unmethylated in a normal control sample (e.g., a DNA sample extracted from a non-cancerous cell or tissue sample or a DNA sample treated for methylation of DNA residues), and in certain embodiments, at least 2, 3, 4, 5, 6, 7, 8, 9, 10 or more Cs may be methylated, wherein the Cs at these positions in the control DNA sample are unmethylated.

In embodiments, a variety of different methods can be used to detect DNA methylation changes. Methods for detecting DNA methylation include, for example, methylation-sensitive restriction endonuclease (MSRE) assays using southern or polymerase chain reaction (PCR) analysis, methylation-specific or methylation-sensitive PCR (MS-PCR), methylation-sensitive single nucleotide primer extension (Ms-SnuPE), high-resolution melting (HRM) analysis, bisulfite sequencing, pyrosequencing, methylation-specific single-strand conformation analysis (MS-SSCA), combined bisulfite restriction analysis (COBRA), methylation-specific denaturing gradient gel electrophoresis (MS-DGGE), methylation-specific melting curve analysis (MS-MCA), methylation-specific denaturing high-performance liquid chromatography (MS-DHPLC), and methylation-specific microarray (MSO). These assays can be PCR analysis, quantitative analysis using fluorescent markers, or southern blot analysis.

As used herein, a “methylation assay” refers to any assay that determines the methylation status of one or more CpG dinucleotide sequences within a DNA sequence.

As used herein, “detection” refers to any process of observing a marker or marker change (e.g., a change in marker methylation status or expression level of a nucleic acid or protein sequence) in a sample, regardless of whether the marker or marker change is actually detected. In other words, the behavior of detecting a sample for a marker or a change in a marker is “detection,” even if the marker is determined to be absent or below a sensitivity level. Detection can be a quantitative, semi-quantitative, or non-quantitative observation and can be based on the comparison to one or more control samples. It is understood that detecting liver cancer as disclosed herein includes detecting precancerous cells that have begun to develop into liver cancer cells, or are about to develop into liver cancer cells, or have an increased propensity to develop into liver cancer cells. Detecting liver cancer may also include detecting the potential mortality possibility or the possible prognosis of the disease condition.

As used herein, the terms “homology,” “identity,” and “similarity” refer to the sequence similarity between two nucleic acid molecules. Positions within each sequence can be compared to determine “homology,” “identity,” or “similarity,” and the sequences can be aligned for comparison purposes. When equivalent positions in the compared sequences are occupied by the same base, the molecules are identical at that position; when equivalent sites are occupied by the same or similar amino acid residues (e.g., similar in spatial properties or charged properties), the molecules can be said to be homologous (similar) at that position. The expression of homology/similarity or identity percentage refers to a function of the number of identical or similar amino acids at positions shared by the compared sequences. “Unrelated” or “non-homologous” sequences share less than 40% identity, preferably less than 25% identity to the sequences of the present application. When comparing two sequences, the absence of residues (amino acid or nucleic acid) or the presence of extra residues also reduces identity and homology/similarity. In a specific embodiment, two or more sequences or subsequences are considered to be substantially or significantly homologous, similar or identical if their sequences have about 60%, or about 65%, 70%, 75%, 80%, 85%, 90%, 91%, 92%, 93%, 94%, 95%, 96%, 97%, 98%, 99% or more identity over a specified region, when comparing and aligning for maximum correspondence over a comparison window or designated region, as determined using the BLAST or BLAST 2.0 sequence comparison algorithm with the default parameters described below, or by manual alignment and visual inspection, for example, as provided online by the National Center for Biotechnology Information (NCBI). This definition also relates to or can be used to test the complement of a sequence. Thus, to the extent permitted by the context herein, for example, a nucleotide sequence that is complementary to a specified target sequence or a variant thereof per se is considered as “similar” to the target sequence if the nucleotide sequence can be predicted to occur naturally in a DNA duplex, or can occur naturally as one or both of the complementary strands, and references to “similar” nucleic acid sequences include single-stranded sequences, their complements, double-stranded complexes, sequences that are capable of encoding the same or similar polypeptide products, and any permitted variants of any of the foregoing. Instances where similarity must be restricted to the analysis of a single nucleic acid strand sequence may include, for example, the detection and quantification of the expression of specific RNA sequences or coding sequences in cells. The definition also includes sequences with deletions and/or additions, as well as sequences with substitutions. In embodiments, the identity or similarity can be over a region that is at least about 9, 10, 11, 12, 13, 14, 15, 16, 17, 18, 19, 10, 21, 22, 23, 24, 25, or more nucleotides in length, or over a region that is more than about 10, 15, 20, 25, 30, 35, 40, 45, 50, 55, 60, 65, 70, 75, 80, 85, 90, 95, or more than about 100 nucleotides in length.

As used herein, “amplification” refers to the process of generating multiple copies from a specific locus of a nucleic acid, such as genomic DNA or cDNA. Amplification can be achieved using any of a variety of known means including, but not limited to, polymerase chain reaction (PCR), transcription-based amplification, and strand displacement amplification (SDA).

As used herein, the “fluorescence-based real-time PCR” or “real-time fluorescence quantitative PCR” refers to a method in which a fluorescent group is added to the PCR reaction system, the entire PCR process is monitored in real time using the accumulation of fluorescent signals, and finally the unknown template is quantitatively analyzed using a standard curve. In this PCR technology, there is a very important concept, i.e., the cycle threshold, also known as the Ct value. C stands for Cycle, t stands for threshold, and the Ct value means the number of cycles it takes for the fluorescence signal in each reaction tube to reach the set threshold. For example, the fluorescence threshold is set as follows: the fluorescence signals of the first 15 cycles of the PCR reaction are used as the fluorescence background signal, and the default setting of the fluorescence threshold is 10 times the standard deviation of the fluorescence signals of cycles 3-15.

As used herein, “cut-off value of real-time PCR” refers to a critical Ct value for determining whether a sample is positive or negative for a certain biomarker. According to certain specific embodiments of the present application, “a critical Ct value (Cut Off value) is obtained based on a certain amount of sample data and statistical processing”, and the critical Ct value may vary depending on the requirements of desired sensitivity or specificity.

As used herein, “sensitivity” refers to the proportion of cancer detected from a certain cancer sample, and its calculation formula is: sensitivity=(detected cancer/all cancers), while “specificity” refers to the proportion of normal detected in a certain normal human sample, and its calculation formula is that specificity=(detected negative/total negative).

As used herein, a “label” or “detectable moiety” is a composition detectable by spectroscopic, photochemical, biochemical, immunochemical, chemical or other physical means. For example, useful labels include 32P, fluorescent dyes, electron-dense reagents, enzymes (e.g., those commonly used in ELISA), biotin, digoxigenin, or haptens and proteins that can be made detectable, for example, by incorporating a radioactive label into the peptide or an antibody that is used to detect the specific reaction with peptide.

Nucleic acid molecules can be detected using a variety of different methods. The detection methods of nucleic acid include, for example, PCR and nucleic acid hybridization (e.g., Southern blotting, Northern blotting, or in situ hybridization). Specifically, oligonucleotides (e.g., oligonucleotide primers) capable of amplifying a target nucleic acid can be used in a PCR reaction. PCR methods generally include the following steps: obtaining a sample, isolating nucleic acid (e.g., DNA, RNA, or both) from the sample, and contacting the nucleic acid with one or more oligonucleotide primers that specifically hybridize to the template nucleic acid under conditions such that amplification of the template nucleic acid occurs. In the presence of template nucleic acid, amplification products are produced. The conditions for nucleic acid amplification and detection of amplification products are known to those skilled in the art. A variety of modifications to the basic PCR technique have been developed, including, but not limited to, anchored PCR, RACE PCR, RT-PCR, and ligase chain reaction (LCR). The primer pairs in an amplification reaction must anneal to opposite strands of the template nucleic acid and should maintain an appropriate distance from each other to allow the polymerase to efficiently polymerize across the region and to allow easy detection of the amplified product, for example, using electrophoresis. For example, computer programs such as OLIGO (Molecular Biology Insights Inc., Cascade, Colo.) can be used to design oligonucleotide primers to help design primers with similar melting temperatures. Typically, oligonucleotide primers are 9-30, or 40, or 50 nucleotides in length (e.g., 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length), but oligonucleotide primers can be longer or shorter so long as appropriate amplification conditions are used.

Detection of the amplification product or hybridization complex is typically achieved using a detectable label. The term “label,” when referring to nucleic acids, is intended to include direct labeling of nucleic acids by coupling (i.e., physically linking) a detectable substance to the nucleic acid, as well as indirect labeling of nucleic acids by reaction with another reagent that is directly labeled with a detectable substance. Detectable substances include various enzymes, prosthetic groups, fluorescent materials, luminescent materials, bioluminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, or phycoerythrin; examples of luminescent materials include luminol; examples of bioluminescent materials include luciferase, luciferin, and aequorin. Examples of indirect labeling include end-labeling of a nucleic acid with biotin, such that the nucleic acid can be detected using fluorescently labeled streptavidin.

On the one hand, the present application provides a composition for detecting liver cancer in vitro, the composition comprising a nucleic acid for detecting the methylation status within a target sequence of a target gene, wherein the methylation status of the target gene is characterized by the methylation of the target sequence of the target gene, wherein the target gene is one or more of a CHFR gene, an SPINT2 gene, an RNF135 gene, a CHST2 gene, an SPDYA gene, a PAX5 gene, a VASH2 gene, a TSPYL5 gene and an EMX1 gene.

Among them, the CHFR gene (Checkpoint with forkhead and ring finger domains) is a mitotic stress checkpoint gene that has been cloned and located on chromosome 12q24.33. In mammalian cells exposed to drugs that disrupt microtubule structure, such as nocodazole or paclitaxel, the CHFR protein mediates a delay in entry into metaphase. Its microscopic hallmark is delayed chromosome condensation and cell cycle progression is delayed until cellular damage is repaired. Furthermore, CHFR promotes cell survival in response to mitotic stress.

The SPINT2 gene (Homo sapiens serine peptidase inhibitor, Kunitz type 2, SPINT2) encodes a transmembrane protein in the serine protease inhibitor. This gene inhibits the HGF/MET signaling pathway by inhibiting the activation of HGF and belongs to tumor suppressor gene. In addition, the SPINT2 gene can also inhibit the activity of hepatocyte growth factor by inhibiting the activation of hepatocyte growth activator. Hepatocyte growth factor can stimulate tumor cell growth and metastasis, and at the same time, stimulate tumor angiogenesis, thus plays an important role in the occurrence and development of tumors. Studies have shown that the SPINT2 gene is regulated by methylation and its expression is downregulated in tissues such as liver cancer, renal cell carcinoma, ovarian cancer, and glioma. SPINT2 is upregulated in breast cancer, and elevated SPINT2 expression is significantly associated with poor prognosis. This study found that SPINT2 is lowly expressed in most gastric cancer cell lines and gastric cancer tissues, and low SPINT2 expression is correlated with clinical indicators of gastric cancer patients (such as the degree of tumor cell differentiation and whether it has metastasized).

The RNF135 gene (Homo sapiens ring finger protein 135) is an E3 ubiquitin ligase containing a ring finger domain, and is involved in the regulation of gene transcription, translation, cell adhesion, epithelial development and cell cycle regulation. RING finger protein 135 (RNF135) consists of an N-terminal RING finger domain and a C-terminal SPRY and PRY motif 15. It belongs to the ring finger protein family E3 ubiquitin ligase and is located on chromosome 17q11.2. This gene ubiquitinates RIG-I in the early stages of viral infection and promotes its signal transduction, thereby producing interferon-β. This process plays a crucial role in a wide range of biological processes in development and disease pathogenesis. Studies have shown that the RNF135 gene is downregulated in Schwann cells of malignant peripheral nerve sheath tumors, and downregulation of RNF135 expression can cause inactivation of the Erk pathway in U87 and U251 glioma cells (the Erk pathway is involved in the regulation of cell cycle progression, cell growth, proliferation and migration), thereby inhibiting cell growth and migration.

The CHST2 gene (GlcNAc6ST-2, HEC-GlcNAc6ST) belongs to the carbohydrate 6-O-sulfur transferase family, which is involved in transferring sulfur groups to N-acetylglucosamine residues of galactose, N-acetylgalactosamine or other glycoproteins. Studies have shown that in normal tissues, CHST2 mRNA is only expressed in lymph node endothelial cells and liver. It is believed to be involved in the assembly of L-selectin ligands in lymph node endothelial cells. The binding of this ligand to L-selectin is the first step in lymphocyte homing, namely the adhesion of lymphocytes to lymphatic endothelial cells. In the analysis of gene expression profiles and cluster analysis of osteosarcoma cell lines with different metastatic potentials, it was found that EREG genes were commonly downregulated and CHST2 genes were commonly upregulated.

The SPDYA (SPY1) gene (Homo sapiens speedy/RINGO cell cycle regulator family member A, SPDYA (SPY1)) is a cell cycle protein and belongs to the Speedy/Ringo family of cell cycle regulators. This gene can promote cell proliferation and survival by activating cyclin-dependent kinase-2. In late G1 phase and throughout S phase, the expression of Spyl enhances CDK2-dependent degradation of p27kip1 by directly promoting the degradation of p27. Studies have confirmed that Spyl is expressed in a variety of human tissues. Overexpression of this gene promotes G1-S phase progression, p27kip degradation and cell proliferation by activating CDK2. The PAX5 gene (paired box protein 5) is a highly conserved gene in evolution and can encode B cell-specific activator protein (BSAP). Studies have shown that B cell differentiation and development is an orderly dynamic transcriptional regulation process. The differentiation and development of B cells play an important role in immune regulation and immune response. Transcription factors affect the directional differentiation and development of B cells. In particular, the transcription factor Pax5 plays a key regulatory role.

The VASH2 gene (Homo sapiens vasohibin 2, VASH2) is a member of the angiostatin family protein and can participate in the regulation of angiogenesis and EMT pathways by interacting with vascular endothelial growth factor (VEGF) family proteins. Studies have shown that VASH2 is associated with the proliferation, metastasis and invasion of various human tumors. Therefore, VASH2 can activate the TGF-β signaling pathway, participate in the regulation of EMT process, and thus enhance the migration and invasion ability of ovarian cancer and breast cancer cells.

The TSPYL5 gene (Homo sapiens TSPY-like 5, TSPYL5) is a member of the TSPYL family. Studies have shown that its expression is lost or downregulated in many tumors. It acts on ubiquitin-specific protease 7 to reduce the level of P53 protein, thereby inhibiting the activity of P53 target genes. Meanwhile, TSPYL5 exhibits high methylation of the promoter (nearly 100%) in primary gliomas and its expression is downregulated, but there is almost no methylation in normal brain tissues.

The EMX1 gene (Homo sapiens empty spiracles homeobox 1) is a member of the EMX transcription factor family. It may be involved in regulating various biological processes, such as cell proliferation, migration, and differentiation during brain and neural crest development. EMX1 plays a tumor suppressor role in sarcomas by inhibiting the activity of stem cell regulatory genes and effectors of the canonical Wnt pathway. The studies showed that overexpression of EMX1 reduced tumorigenicity, while knocking down the levels of these genes enhanced these properties. Moreover, restoration of EMX1 expression levels inhibits cell proliferation and invasion.

In a specific embodiment, the target gene is one or more of a CHFR gene, an SPINT2 gene, an RNF135 gene, an SPDYA gene, a PAX5 gene, and a VASH2 gene.

In a specific embodiment, the target genes are the six genes which are a CHFR gene, an SPINT2 gene, an RNF135 gene, an SPDYA gene, a PAX5 gene, and a VASH2 gene.

In a specific embodiment, the target gene is one, two or three of an RNF135 gene, a CHFR gene and a PAX5 gene.

In a specific embodiment, the target genes are the three genes which are an RNF135 gene, a CHFR gene and a PAX5 gene.

The present application provides a group of target sequences of target genes that exhibit abnormal methylation in liver cancer, including target sequences of one or more of a CHFR gene, an SPINT2 gene, an RNF135 gene, a CHST2 gene, an SPDYA gene, a PAX5 gene, a VASH2 gene, a TSPYL5 gene and an EMX1 gene. The target sequence of the CHFR gene is shown as any one of SEQ ID NOs: 1-4 or comprises a sequence shown as any one of SEQ ID NOs: 1-4, the target sequence of the SPINT2 gene is shown as any one of SEQ ID NOs: 5-8 or comprises a sequence shown as any one of SEQ ID NOs: 5-8, the target sequence of the RNF135 gene is shown as any one of SEQ ID NOs: 9-12 or comprises a sequence shown as any one of SEQ ID NOs: 9-12, the target sequence of the CHST2 gene is shown as any one of SEQ ID NOs: 13-16 or comprises a sequence shown as any one of SEQ ID NOs: 13-16, and the target sequence of the SPDYA gene is shown as SEQ ID NOs: 17-20 or comprises a sequence shown as any one of SEQ ID NOs: 17-20, the target sequence of the PAX5 gene is shown as any one of SEQ ID NOs: 21-24 or comprises a sequence shown as any one of SEQ ID NOs: 21-24, the target sequence of the VASH2 gene is shown as any one of SEQ ID NOs: 25-28 or comprises a sequence shown as any one of SEQ ID NOs: 25-28, the target sequence of the TSPYL5 gene is shown as any one of SEQ ID NOs: 29-32 or comprises a sequence shown as any one of SEQ ID NOs: 29-32, and the target sequence of the EMX1 gene is shown as any one of SEQ ID NOs: 33-36 or comprises a sequence shown as any one of SEQ ID NOs: 33-36.

The target sequence (5′-3′) of the RNF135 gene is as follows:

SEQ ID NO:1
TGGTTTCCCAGGGCAAGAGGCCGCCACTGAAGGTCAGCTCTGTCTTTCCAGCTGGGCAC
TGGAGGCTCTAAGTCTGTTTTCAGCAGGATCGGAGGAAGGAGACGGGGTGGCGCCAAG
GAAGGAGGAGAAAAGGCGGCCGAGAAAAGGAGGAGGGCAAGGGGAAGAGGAAGGGC
GAGGGAGGAGCCTGAGGAGACTCGCCCGGCTCAACCCCGACGTCCGCGCCCCGGCCGC
CTGTTGGCCATGGCGGGCCTGGGCCTGGGCTCCGCCGTTCCCGTGTGGCTGGCCGAGGA
CGACCTCGGCTGCATCATCTGCCAGGGGCTGCTGGACTGGCCCGCCACGCTGCCCTGCG
GCCACAGCTTCTGCCGCCACTGCCTGGAGGCCCTGTGGGGCGCCCGCGACGCCCGCCGC
TGGGCCTGCCCCACTTGCCGCCAGGGCGCCGCGCAGCAGCCGCACCTGCGGAAGAACA
CGCTACTGCAGGACCTGGCCGACAAGTACCGCCGCGCCGCACGCGAGATACAGGCGGG
CTCCGACCCTGCCCACTGCCCCTGCCCGGGCTCCAGTTCCCTCTCCAGCG

The sequence (5′-3′) after the bisulfite treatment of the target sequence of the RNF135 gene is as follows:

SEQ ID NO:2
TGGTTTTTTAGGGTAAGAGGTCGTTATTGAAGGTTAGTTTTGTTTTTTTAGTTGGGTATTG
GAGGTTTTAAGTTTGTTTTTAGTAGGATCGGAGGAAGGAGACGGGGTGGCGTTAAGGAA
GGAGGAGAAAAGGCGGTCGAGAAAAGGAGGAGGGTAAGGGGAAGAGGAAGGGCGAG
GGAGGAGTTTGAGGAGATTCGTTCGGTTTAATTTCGACGTTCGCGTTTCGGTCGTTTGTT
GGTTATGGCGGGTTTGGGTTTGGGTTTCGTCGTTTTCGTGTGGTTGGTCGAGGACGATTT
CGGTTGTATTATTTGTTAGGGGTTGTTGGATTGGTTCGTTACGTTGTTTTGCGGTTATAGT
TTTTGTCGTTATTGTTTGGAGGTTTTGTGGGGCGTTCGCGACGTTCGTCGTTGGGTTTGTT
TTATTTGTCGTTAGGGCGTCGCGTAGTAGTCGTATTTGCGGAAGAATACGTTATTGTAGG
ATTTGGTCGATAAGTATCGTCGCGTCGTACGCGAGATATAGGCGGGTTTCGATTTTGTTT
ATTGTTTTTGTTCGGGTTTTAGTTTTTTTTTTAGCG

The complementary sequence (5′-3′) of the target sequence of the RNF135 gene is as follows:

SEQ ID NO:3
CGCTGGAGAGGGAACTGGAGCCCGGGCAGGGGCAGTGGGCAGGGTCGGAGCCCGCCTG
TATCTCGCGTGCGGCGCGGCGGTACTTGTCGGCCAGGTCCTGCAGTAGCGTGTTCTTCCG
CAGGTGCGGCTGCTGCGCGGCGCCCTGGCGGCAAGTGGGGCAGGCCCAGCGGCGGGCG
TCGCGGGCGCCCCACAGGGCCTCCAGGCAGTGGCGGCAGAAGCTGTGGCCGCAGGGCA
GCGTGGCGGGCCAGTCCAGCAGCCCCTGGCAGATGATGCAGCCGAGGTCGTCCTCGGCC
AGCCACACGGGAACGGCGGAGCCCAGGCCCAGGCCCGCCATGGCCAACAGGCGGCCGG
GGCGCGGACGTCGGGGTTGAGCCGGGCGAGTCTCCTCAGGCTCCTCCCTCGCCCTTCCT
CTTCCCCTTGCCCTCCTCCTTTTCTCGGCCGCCTTTTCTCCTCCTTCCTTGGCGCCACCCC
GTCTCCTTCCTCCGATCCTGCTGAAAACAGACTTAGAGCCTCCAGTGCCCAGCTGGAAA
GACAGAGCTGACCTTCAGTGGCGGCCTCTTGCCCTGGGAAACCA

The sequence (5′-3′) after the bisulfite treatment of the complementary sequence of the target sequence of the RNF135 gene is as follows:

SEQ ID NO:4
CGTTGGAGAGGGAATTGGAGTTCGGGTAGGGGTAGTGGGTAGGGTCGGAGTTCGTTTGT
ATTTCGCGTGCGGCGCGGCGGTATTTGTCGGTTAGGTTTTGTAGTAGCGTGTTTTTTCGT
AGGTGCGGTTGTTGCGCGGCGTTTTGGCGGTAAGTGGGGTAGGTTTAGCGGCGGGCGTC
GCGGGCGTTTTATAGGGTTTTTAGGTAGTGGCGGTAGAAGTTGTGGTCGTAGGGTAGCG
TGGCGGGTTAGTTTAGTAGTTTTTGGTAGATGATGTAGTCGAGGTCGTTTTCGGTTAGTT
ATACGGGAACGGCGGAGTTTAGGTTTAGGTTCGTTATGGTTAATAGGCGGTCGGGGCGC
GGACGTCGGGGTTGAGTCGGGCGAGTTTTTTTAGGTTTTTTTTTCGTTTTTTTTTTTTTTTT
TGTTTTTTTTTTTTTTTCGGTCGTTTTTTTTTTTTTTTTTTTGGCGTTATTTCGTTTTTTTTTT
TCGATTTTGTTGAAAATAGATTTAGAGTTTTTAGTGTTTAGTTGGAAAGATAGAGTTGAT
TTTTAGTGGCGGTTTTTTGTTTTGGGAAATTA

The target sequence (5′-3′) of the CHFR gene is as follows:

SEQ ID NO:5
AGAGGGGGGGGTCGGGGCGGAGAGCCGCGCCCAAAGGCAATGGGAGCCGCACGCTGCT
AGGCAACATGCTGTCTCCCGGCAACGTAGAGGCGGGGAATTCGCCAACGTGTCTGAAA
CAGACAACAATTTTAGCCAACGGCTGCGTGGGCCTTGGGCGTCCTGGCCAATGGGAATA
GTGAGGGGCCTGTCACTAAGAAACCTGTCCCTGAGCAACGCATAGGAGGGGTGGAACC
CAGCAGCGCTGACTGAGCCGCCGCGAAGTGCCGGAACGTATTACCTGGGAGCCAAATTT
CAGAGCGTCGTTTGTCTTCCCAAGCCCACCAGCTTTCAGCGTTCTCCAGCCTCGCCACAC
CCCCGCCTCGGGCCGTCCCCGCCCAGACGCTTGTCCCCGCGAGAAGCTGAGCGCACTGA
AGTGAGCCCGGCGGCTGCCGCCGGAGGAGGAGCCCGGCTCTGGACACGGCCGTCA

The sequence (5′-3′) after the bisulfite treatment of the target sequence of the CHFR gene is as follows:

SEQ ID NO:6
AGAGGGGGGGGTCGGGGCGGAGAGTCGCGTTTAAAGGTAATGGGAGTCGTACGTTGTT
AGGTAATATGTTGTTTTTCGGTAACGTAGAGGGGGGAATTCGTTAACGTGTTTGAAAT
AGATAATAATTTTAGTTAACGGTTGCGTGGGTTTTGGGCGTTTTGGTTAATGGGAATAGT
GAGGGGTTTGTTATTAAGAAATTTGTTTTTGAGTAACGTATAGGAGGGGTGGAATTTAG
TAGCGTTGATTGAGTCGTCGCGAAGTGTCGGAACGTATTATTTGGGAGTTAAATTTTAG
AGCGTCGTTTGTTTTTTTAAGTTTATTAGTTTTTAGCGTTTTTTAGTTTCGTTATATTTTCG
TTTCGGGTCGTTTTCGTTTAGACGTTTGTTTTCGCGAGAAGTTGAGCGTATTGAAGTGAG
TTCGGCGGTTGTCGTCGGAGGAGGAGTTCGGTTTTGGATACGGTCGTTA

The complementary sequence (5′-3′) of the target sequence of the CHFR gene is as follows:

SEQ ID NO:7
TGACGGCCGTGTCCAGAGCCGGGCTCCTCCTCCGGCGGCAGCCGCCGGGCTCACTTCAG
TGCGCTCAGCTTCTCGCGGGGACAAGCGTCTGGGCGGGGACGGCCCGAGGCGGGGGTG
TGGCGAGGCTGGAGAACGCTGAAAGCTGGTGGGCTTGGGAAGACAAACGACGCTCTGA
AATTTGGCTCCCAGGTAATACGTTCCGGCACTTCGCGGCGGCTCAGTCAGCGCTGCTGG
GTTCCACCCCTCCTATGCGTTGCTCAGGGACAGGTTTCTTAGTGACAGGCCCCTCACTAT
TCCCATTGGCCAGGACGCCCAAGGCCCACGCAGCCGTTGGCTAAAATTGTTGTCTGTTT
CAGACACGTTGGCGAATTCCCCGCCTCTACGTTGCCGGGAGACAGCATGTTGCCTAGCA
GCGTGCGGCTCCCATTGCCTTTGGGCGCGGCTCTCCGCCCCGACCCGCCCCTCT

The sequence (5′-3′) after the bisulfite treatment of the complementary sequence of the target sequence of the CHFR gene is as follows:

SEQ ID NO: 8
TGACGGTCGTGTTTAGAGTCGGGTTTTTTTTTCGGCGGTAGTCGTCGGG
TTTATTTTAGTGCGTTTAGTTTTTCGCGGGGATAAGCGTTTGGGCGGGG
ACGGTTCGAGGCGGGGGTGTGGCGAGGTTGGAGAACGTTGAAAGTTGGT
GGGTTTGGGAAGATAAACGACGTTTTGAAATTTGGTTTTTAGGTAATAC
GTTTCGGTATTTCGCGGCGGTTTAGTTAGCGTTGTTGGGTTTTATTTTT
TTTATGCGTTGTTTAGGGATAGGTTTTTTAGTGATAGGTTTTTTATTAT
TTTTATTGGTTAGGACGTTTAAGGTTTACGTAGTCGTTGGTTAAAATTG
TTGTTTGTTTTAGATACGTTGGCGAATTTTTCGTTTTTACGTTGTCGGG
AGATAGTATGTTGTTTAGTAGCGTGCGGTTTTTATTGTTTTTGGGCGCG
GTTTTTCGTTTCGATTCGTTTTTTT

The target sequence (5′-3′) of the PAX5 gene is as follows:

SEQ ID NO: 9
TAATTCAAGCCTTCCGCTCCCCCGCCGAGCTGGGGTAGCTGATCACTGA
GCTGAAACTAAACGTTTTAGGTGGAAAAAAAGCGTCCGAAGGCACCGTG
AAATGATTAAGGAACTAAAGAGCTTCTCGCCATGTGAGATCATGTCCTG
TTCTCGCCAACATCACAAGATGTCCCCAGACACGCCGCGCCCCCAGCGC
GCCGCCCCACACTGCCGGCCCGGAGCGAGGAAAGGGTAGGCGCTGCGCG
GCCGGGCCTGCTCAGCGCGCCAGACGTGGCGGACCCGGCCCGGCCGGAG
TAGAGCGGGAAGCCGGGAGAGCAGCAGTGCTGCTGCCGCGCCGCCCCAG
ACTTTTATAGGGGTTGGGGGGAGGGAAGGAAGGCTTCAGCCTGCGCCGG
GCGCTAGCCAGCGCACCTACGGGAAG

The sequence (5′-3′) after the bisulfite treatment of the target sequence of the PAX5 gene is as follows:

SEQ ID NO: 10
TAATTTAAGTTTTTCGTTTTTTCGTCGAGTTGGGGTAGTTGATTATTGA
GTTGAAATTAAACGTTTTAGGTGGAAAAAAAGCGTTCGAAGGTATCGTG
AAATGATTAAGGAATTAAAGAGTTTTTCGTTATGTGAGATTATGTTTTG
TTTTCGTTAATATTATAAGATGTTTTTAGATACGTCGCGTTTTTAGCGC
GTCGTTTTATATTGTCGGTTCGGAGCGAGGAAAGGGTAGGCGTTGCGCG
GTCGGGTTTGTTTAGCGCGTTAGACGTGGCGGATTCGGTTCGGTCGGAG
TAGAGCGGGAAGTCGGGAGAGTAGTAGTGTTGTTGTCGCGTCGTTTTAG
ATTTTTATAGGGGTTGGGGGGAGGGAAGGAAGGTTTTAGTTTGCGTCGG
GCGTTAGTTAGCGTATTTACGGGAAG

The complementary sequence (5′-3′) of the target sequence of the PAX5 gene is as follows:

SEQ ID NO: 11
CTTCCCGTAGGTGCGCTGGCTAGCGCCCGGCGCAGGCTGAAGCCTTCCT
TCCCTCCCCCCAACCCCTATAAAAGTCTGGGGCGGCGCGGCAGCAGCAC
TGCTGCTCTCCCGGCTTCCCGCTCTACTCCGGCCGGGCCGGGTCCGCCA
CGTCTGGCGCGCTGAGCAGGCCCGGCCGCGCAGCGCCTACCCTTTCCTC
GCTCCGGGCCGGCAGTGTGGGGCGGCGCGCTGGGGGCGCGGCGTGTCTG
GGGACATCTTGTGATGTTGGCGAGAACAGGACATGATCTCACATGGCGA
GAAGCTCTTTAGTTCCTTAATCATTTCACGGTGCCTTCGGACGCTTTTT
TTCCACCTAAAACGTTTAGTTTCAGCTCAGTGATCAGCTACCCCAGCTC
GGCGGGGGAGCGGAAGGCTTGAATTA

The sequence (5′-3′) after the bisulfite treatment of the complementary sequence of the target sequence of the PAX5 gene is as follows:

SEQ ID NO: 12
TTTTTCGTAGGTGCGTTGGTTAGCGTTCGGCGTAGGTTGAAGTTTTTTT
TTTTTTTTTTTAATTTTTATAAAAGTTTGGGGCGGCGCGGTAGTAGTAT
TGTTGTTTTTTCGGTTTTTCGTTTTATTTCGGTCGGGTCGGGTTCGTTA
CGTTTGGCGCGTTGAGTAGGTTCGGTCGCGTAGCGTTTATTTTTTTTTC
GTTTCGGGTCGGTAGTGTGGGGCGGCGCGTTGGGGGCGCGGCGTGTTTG
GGGATATTTTGTGATGTTGGCGAGAATAGGATATGATTTTATATGGCGA
GAAGTTTTTTAGTTTTTTAATTATTTTACGGTGTTTTCGGACGTTTTTT
TTTTATTTAAAACGTTTAGTTTTAGTTTAGTGATTAGTTATTTTAGTTC
GGCGGGGGAGCGGAAGGTTTGAATTA

The target sequence (5′-3′) of the SPINT2 gene is as follows:

SEQ ID NO: 13
CGCCCCGCCCCGCCAGGTTCTGTTGGGGGCGAGGCCCGCGCAAGCCCCG
CCTCTTCCCCGGCACCAGGGGGGGGCCCAGGTGCGCCCAGGGCCGGGGA
GCGGCCGCGCAGGTGCCTGCCCTTTGCGCCTGCGCCCAGCTCGCCCTGC
CTAGCCAGGTGCGCCCCGCCCCCTGCCTGCCCGGCCACCTTCGGGAGCC
GCTTCCAATAGGCGTTCGCCATTGGCTCTGGCGACCTCCGCGCGTTGGG
AGGTGTAGCGCGGCTCTGAACGCGCTGAGGGCCGTTGAGTGTCGCAGGC
GGCGAGGGCGCGAGTGAGGAGCAGACCCAGGCATCGCGCGCCGAGAAGG
CCGGGCGTCCCCACACTGAAGGTCCGGAAAGGCGACTTCCGGGGGCTTT
GGCACCTGGCGGACCCTCCCGGAGCGTCGGCACCTGAACGCGAGGCGCT
CCATTGCGCGTGCGCGTTGAGGGGCT

The sequence (5′-3′) after the bisulfite treatment of the target sequence of the SPINT2 gene is as follows:

SEQ ID NO: 14
CGTTTCGTTTCGTTAGGTTTTGTTGGGGGCGAGGTTCGCGTAAGTTTCG
TTTTTTTTTCGGTATTAGGGGGGGGTTTAGGTGCGTTTAGGGTCGGGGA
GCGGTCGCGTAGGTGTTTGTTTTTTGCGTTTGCGTTTAGTTCGTTTTGT
TTAGTTAGGTGCGTTTCGTTTTTTGTTTGTTCGGTTATTTTCGGGAGTC
GTTTTTAATAGGCGTTCGTTATTGGTTTTGGCGATTTTCGCGCGTTGGG
AGGTGTAGCGCGGTTTTGAACGCGTTGAGGGTCGTTGAGTGTCGTAGGC
GGCGAGGGCGCGAGTGAGGAGTAGATTTAGGTATCGCGCGTCGAGAAGG
TCGGGCGTTTTTATATTGAAGGTTCGGAAAGGCGATTTTCGGGGGTTTT
GGTATTTGGCGGATTTTTTCGGAGCGTCGGTATTTGAACGCGAGGCGTT
TTATTGCGCGTGCGCGTTGAGGGGTT

The complementary sequence (5′-3′) of the target sequence of the SPINT2 gene is as follows:

SEQ ID NO: 15
AGCCCCTCAACGCGCACGCGCAATGGAGCGCCTCGCGTTCAGGTGCCGA
CGCTCCGGGAGGGTCCGCCAGGTGCCAAAGCCCCCGGAAGTCGCCTTTC
CGGACCTTCAGTGTGGGGACGCCCGGCCTTCTCGGCGCGCGATGCCTGG
GTCTGCTCCTCACTCGCGCCCTCGCCGCCTGCGACACTCAACGGCCCTC
AGCGCGTTCAGAGCCGCGCTACACCTCCCAACGCGCGGAGGTCGCCAGA
GCCAATGGCGAACGCCTATTGGAAGCGGCTCCCGAAGGTGGCCGGGCAG
GCAGGGGGCGGGGCGCACCTGGCTAGGCAGGGCGAGCTGGGCGCAGGCG
CAAAGGGCAGGCACCTGCGCGGCCGCTCCCCGGCCCTGGGCGCACCTGG
GCCCGCCCCTGGTGCCGGGGAAGAGGCGGGGCTTGCGCGGGCCTCGCCC
CCAACAGAACCTGGCGGGGGGGGCG

The sequence (5′-3′) after the bisulfite treatment of the complementary sequence of the target sequence of the SPINT2 gene is as follows:

SEQ ID NO: 16
AGTTTTTTAACGCGTACGCGTAATGGAGCGTTTCGCGTTTAGGTGTCGA
CGTTTCGGGAGGGTTCGTTAGGTGTTAAAGTTTTCGGAAGTCGTTTTTT
CGGATTTTTAGTGTGGGGACGTTCGGTTTTTTCGGCGCGCGATGTTTGG
GTTTGTTTTTTATTCGCGTTTTCGTCGTTTGCGATATTTAACGGTTTTT
AGCGCGTTTAGAGTCGCGTTATATTTTTTAACGCGCGGAGGTCGTTAGA
GTTAATGGCGAACGTTTATTGGAAGCGGTTTTCGAAGGTGGTCGGGTAG
GTAGGGGGCGGGGCGTATTTGGTTAGGTAGGGCGAGTTGGGCGTAGGCG
TAAAGGGTAGGTATTTGCGCGGTCGTTTTTCGGTTTTGGGCGTATTTGG
GTTCGTTTTTGGTGTCGGGGAAGAGGCGGGGTTTGCGCGGGTTTCGTTT
TTAATAGAATTTGGCGGGGCGGGGCG

The target sequence (5′-3′) of the SPDYA gene is as follows:

SEQ ID NO: 17
AAGGGAAGTAAACGGCCCCAACGCAAGCCTGACTGCGAGACGTGCCCAA
GGGAGGTAGGTCGAATGAAGAGGGTTGTGTGAGTTTTGCGCGGGCAGGC
GGGATAACGGAGGAGGGAGGCCCGCGGCCGAGGCTCGGGCGGGCGGGGG
GCAGGGAGGGGCGGGGTTCGCCGGCGCGCACTCCCAGGCAGGCCCCGCC
CCCTCGGCCGGCTGTGCGCGCTGATTGGCCCCTGCCGGCCTCGCGCTCC
CTCGCTCCGGGTTGGCGGGAGACCTTAGAGCGGGTACCGCTGCTGGCTA
GCGACCGACGAGCAACCGTCTGAGGCCAGGAGCGCTGCGACGGAGCCTT
GACCGCCGTTGCCCGGCCCTCTCCCGCGCAGCCCCGGGCTTCCGCAGGT
ACCTGTGCTCGCCCCCGGGAAGGGGCCTC

The sequence (5′-3′) after the bisulfite treatment of the target sequence of the SPDYA gene is as follows:

SEQ ID NO: 18
AAGGGAAGTAAACGGTTTTAACGTAAGTTTGATTGCGAGACGTGTTTAA
GGGAGGTAGGTCGAATGAAGAGGGTTGTGTGAGTTTTGCGCGGGTAGGC
GGGATAACGGAGGAGGGAGGTTCGCGGTCGAGGTTCGGGCGGGCGGGGG
GTAGGGAGGGGCGGGGTTCGTCGGCGCGTATTTTTAGGTAGGTTTCGTT
TTTTCGGTCGGTTGTGCGCGTTGATTGGTTTTTGTCGGTTTCGCGTTTT
TTCGTTTCGGGTTGGCGGGAGATTTTAGAGCGGGTATCGTTGTTGGTTA
GCGATCGACGAGTAATCGTTTGAGGTTAGGAGCGTTGCGACGGAGTTTT
GATCGTCGTTGTTCGGTTTTTTTTCGCGTAGTTTCGGGTTTTCGTAGGT
ATTTGTGTTCGTTTTCGGGAAGGGGTTTT

The complementary sequence (5′-3′) of the target sequence of the SPDYA gene is as follows:

SEQ ID NO: 19
GAGGCCCCTTCCCGGGGGCGAGCACAGGTACCTGCGGAAGCCCGGGGCT
GCGCGGGAGAGGGCCGGGCAACGGCGGTCAAGGCTCCGTCGCAGCGCTC
CTGGCCTCAGACGGTTGCTCGTCGGTCGCTAGCCAGCAGCGGTACCCGC
TCTAAGGTCTCCCGCCAACCCGGAGCGAGGGAGCGCGAGGCCGGCAGGG
GCCAATCAGCGCGCACAGCCGGCCGAGGGGGCGGGGCCTGCCTGGGAGT
GCGCGCCGGCGAACCCCGCCCCTCCCTGCCCCCCGCCCGCCCGAGCCTC
GGCCGCGGGCCTCCCTCCTCCGTTATCCCGCCTGCCCGCGCAAAACTCA
CACAACCCTCTTCATTCGACCTACCTCCCTTGGGCACGTCTCGCAGTCA
GGCTTGCGTTGGGGCCGTTTACTTCCCTT

The sequence (5′-3′) after the bisulfite treatment of the complementary sequence of the target sequence of the SPDYA gene is as follows:

SEQ ID NO: 20
GAGGTTTTTTTTCGGGGGCGAGTATAGGTATTTGCGGAAGTTCGGGGTT
GCGCGGGAGAGGGTCGGGTAACGGCGGTTAAGGTTTCGTCGTAGCGTTT
TTGGTTTTAGACGGTTGTTCGTCGGTCGTTAGTTAGTAGCGGTATTCGT
TTTAAGGTTTTTCGTTAATTCGGAGCGAGGGAGCGCGAGGTCGGTAGGG
GTTAATTAGCGCGTATAGTCGGTCGAGGGGGCGGGGTTTGTTTGGGAGT
GCGCGTCGGCGAATTTCGTTTTTTTTTGTTTTTCGTTCGTTCGAGTTTC
GGTCGCGGGTTTTTTTTTTTCGTTATTTCGTTTGTTCGCGTAAAATTTA
TATAATTTTTTTTATTCGATTTATTTTTTTTGGGTACGTTTCGTAGTTA
GGTTTGCGTTGGGGTCGTTTATTTTTTTT

The target sequence (5′-3′) of the VASH2 gene is as follows:

SEQ ID NO: 21
CAGCTGTGGCAGAACAGTCAGGGCCTGCCTGACCTCTTGAGTTTTATTG
TCTCCTCTCAACTTGGGCTTAAAGGGACTTTATTAACCATGACTAATAC
CAGAGGATCCAAAGCTGCACCCACTTGTTAATTTTGAGGAATTTTTAAA
TGCGGGATTTAAAAAAGATTTAATTTCACGCATAGGTATGAACAGCCCC
TCATCATCCCAGGTAGAGGATGAACAACTCATCATCCCGGGTAGAGGCA
GGTAAGTGTGTCCAGCGTCCCTCAGGGAATGCTCTTCTGATGCCAAACC
CTCTGCTTCCGCGGGGTCACTTCTCGGGTGGTTTTGTTTCTCCTCTGCT
TCCACAGTTGCAAACCCTCGAAGTGCCACGACCCCTGGGGAAACTCGAT
CTTAAGTCCCATGGCCAAACGCTCCAGCCCGCGGGGCTCTCAGGACACC
ACAGCGATCGGATTTCGCGTGGCCGCCAGGCTCCCCCTACTCCTGTCCC
CTCCCGCCAGAGAACCCCTGGAGCCAATCAGAGCGTCGCGGAGCCCGGG
GTTGTAGAGTCCCGAGCCCGGGACCCGCCCCTCTTTGTTGCGGTGGCCA
ATAGACGCATTCGGAACATCGG

The sequence (5′-3′) after the bisulfite treatment of the target sequence of the VASH2 gene is as follows:

SEQ ID NO: 22
TAGTTGTGGTAGAATAGTTAGGGTTTGTTTGATTTTTTGAGTTTTATTG
TTTTTTTTTAATTTGGGTTTAAAGGGATTTTATTAATTATGATTAATAT
TAGAGGATTTAAAGTTGTATTTATTTGTTAATTTTGAGGAATTTTTAAA
TGCGGGATTTAAAAAAGATTTAATTTTACGTATAGGTATGAATAGTTTT
TTATTATTTTAGGTAGAGGATGAATAATTTATTATTTCGGGTAGAGGTA
GGTAAGTGTGTTTAGCGTTTTTTAGGGAATGTTTTTTTGATGTTAAATT
TTTTGTTTTCGCGGGGTTATTTTTCGGGTGGTTTTGTTTTTTTTTTGTT
TTTATAGTTGTAAATTTTCGAAGTGTTACGATTTTTGGGGAAATTCGAT
TTTAAGTTTTATGGTTAAACGTTTTAGTTCGCGGGGTTTTTAGGATATT
ATAGCGATCGGATTTCGCGTGGTCGTTAGGTTTTTTTTATTTTTGTTTT
TTTTCGTTAGAGAATTTTTGGAGTTAATTAGAGCGTCGCGGAGTTCGGG
GTTGTAGAGTTTCGAGTTCGGGATTCGTTTTTTTTTGTTGCGGTGGTTA
ATAGACGTATTCGGAATATCGG

The complementary sequence (5′-3′) of the target sequence of the VASH2 gene is as follows:

SEQ ID NO: 23
CCGATGTTCCGAATGCGTCTATTGGCCACCGCAACAAAGAGGGGCGGGT
CCCGGGCTCGGGACTCTACAACCCCGGGCTCCGCGACGCTCTGATTGGC
TCCAGGGGTTCTCTGGCGGGAGGGGACAGGAGTAGGGGGAGCCTGGCGG
CCACGCGAAATCCGATCGCTGTGGTGTCCTGAGAGCCCCGCGGGCTGGA
GCGTTTGGCCATGGGACTTAAGATCGAGTTTCCCCAGGGGTCGTGGCAC
TTCGAGGGTTTGCAACTGTGGAAGCAGAGGAGAAACAAAACCACCCGAG
AAGTGACCCCGCGGAAGCAGAGGGTTTGGCATCAGAAGAGCATTCCCTG
AGGGACGCTGGACACACTTACCTGCCTCTACCCGGGATGATGAGTTGTT
CATCCTCTACCTGGGATGATGAGGGGCTGTTCATACCTATGCGTGAAAT
TAAATCTTTTTTAAATCCCGCATTTAAAAATTCCTCAAAATTAACAAGT
GGGTGCAGCTTTGGATCCTCTGGTATTAGTCATGGTTAATAAAGTCCCT
TTAAGCCCAAGTTGAGAGGAGACAATAAAACTCAAGAGGTCAGGCAGGC
CCTGACTGTTCTGCCACAGCTG

The sequence (5′-3′) after the bisulfite treatment of the complementary sequence of the target sequence of the VASH2 gene is as follows:

SEQ ID NO: 24
TCGATGTTTCGAATGCGTTTATTGGTTATCGTAATAAAGAGGGGCGGGT
TTCGGGTTCGGGATTTTATAATTTCGGGTTTCGCGACGTTTTGATTGGT
TTTAGGGGTTTTTTGGCGGGAGGGGATAGGAGTAGGGGGAGTTTGGCGG
TTACGCGAAATTCGATCGTTGTGGTGTTTTGAGAGTTTCGCGGGTTGGA
GCGTTTGGTTATGGGATTTAAGATCGAGTTTTTTTAGGGGTCGTGGTAT
TTCGAGGGTTTGTAATTGTGGAAGTAGAGGAGAAATAAAATTATTCGAG
AAGTGATTTCGCGGAAGTAGAGGGTTTGGTATTAGAAGAGTATTTTTTG
AGGGACGTTGGATATATTTATTTGTTTTTATTCGGGATGATGAGTTGTT
TATTTTTTATTTGGGATGATGAGGGGTTGTTTATATTTATGCGTGAAAT
TAAATTTTTTTTAAATTTCGTATTTAAAAATTTTTTAAAATTAATAAGT
GGGTGTAGTTTTGGATTTTTTGGTATTAGTTATGGTTAATAAAGTTTTT
TTAAGTTTAAGTTGAGAGGAGATAATAAAATTTAAGAGGTTAGGTAGGT
TTTGATTGTTTTGTTATAGTTG

The target sequence (5′-3′) of the CHST2 gene is as follows:

SEQ ID NO: 25
CAAGTATTTTTCTGCAAATTAGAACGGGGCGTTCCCCTCCCCCATCACC
CCAATTCCAGAGGACGTCAATCACAGTGGACGCAGCACCGCGAGCGGAA
ATGCGGAGAGCATCGGGGCTCCGCATGTGGGGCACAATGAAACAAGAGC
AGCCCCGGCCAGAGGTCCAGTAAAGCGGAAAGACGGTCCCGCCCGCACA
CCCCTCCGGAAAACTGAAGCGTCTCCTCTGACCCCCAAGGTCAGAAAGA
TTTATTACTTCCTTCGAGAGGGCGCAGCCACAGACCCACTAGAGAGGGC
GGGCAGGGTGTCGGAAGCCAGAGAACGGCGCTCCGCGTCCTCCCACCGC
TCCAGGGCGCAG

The sequence (5′-3′) after the bisulfite treatment of the target sequence of the CHST2 gene is as follows:

SEQ ID NO: 26
TAAGTATTTTTTTGTAAATTAGAACGGGGCGTTTTTTTTTTTTATTATT
TTAATTTTAGAGGACGTTAATTATAGTGGACGTAGTATCGCGAGCGGAA
ATGCGGAGAGTATCGGGGTTTCGTATGTGGGGTATAATGAAATAAGAGT
AGTTTCGGTTAGAGGTTTAGTAAAGCGGAAAGACGGTTTCGTTCGTATA
TTTTTTCGGAAAATTGAAGCGTTTTTTTTGATTTTTAAGGTTAGAAAGA
TTTATTATTTTTTTCGAGAGGGCGTAGTTATAGATTTATTAGAGAGGGC
GGGTAGGGTGTCGGAAGTTAGAGAACGGCGTTTCGCGTTTTTTTATCGT
TTTAGGGCGTAG

The complementary sequence (5′-3′) of the target sequence of the CHST2 gene is as follows:

SEQ ID NO: 27
CTGCGCCCTGGAGCGGTGGGAGGACGCGGAGCGCCGTTCTCTGGCTTCC
GACACCCTGCCCGCCCTCTCTAGTGGGTCTGTGGCTGCGCCCTCTCGAA
GGAAGTAATAAATCTTTCTGACCTTGGGGGTCAGAGGAGACGCTTCAGT
TTTCCGGAGGGGTGTGCGGGCGGGACCGTCTTTCCGCTTTACTGGACCT
CTGGCCGGGGCTGCTCTTGTTTCATTGTGCCCCACATGCGGAGCCCCGA
TGCTCTCCGCATTTCCGCTCGCGGTGCTGCGTCCACTGTGATTGACGTC
CTCTGGAATTGGGGTGATGGGGGAGGGGAACGCCCCGTTCTAATTTGCA
GAAAAATACTTG

The sequence (5′-3′) after the bisulfite treatment of the complementary sequence of the target sequence of the CHST2 gene is as follows:

SEQ ID NO: 28
TTGCGTTTTGGAGCGGTGGGAGGACGCGGAGCGTCGTTTTTTGGTTTTC
GATATTTTGTTCGTTTTTTTTAGTGGGTTTGTGGTTGCGTTTTTTCGAA
GGAAGTAATAAATTTTTTTGATTTTGGGGGTTAGAGGAGACGTTTTAGT
TTTTCGGAGGGGTGTGCGGGCGGGATCGTTTTTTCGTTTTATTGGATTT
TTGGTCGGGGTTGTTTTTGTTTTATTGTGTTTTATATGCGGAGTTTCGA
TGTTTTTCGTATTTTCGTTCGCGGTGTTGCGTTTATTGTGATTGACGTT
TTTTGGAATTGGGGTGATGGGGGAGGGGAACGTTTCGTTTTAATTTGTA
GAAAAATATTTG

The target sequence (5′-3′) of the TSPYL5 gene is as follows:

SEQ ID NO: 29
CGGACTCGGGCTTTGGCGCGGCCTTTGCCCCGGTTTTTGGCGCGGGAGG
ACTTTCGACCCCGACTTCGGCCGCTCATGGTGGCGGCGGAGGCAGCTTC
AAAGACACGCTGTGACCCTGCGGCTCCTGACGCCAGCTCTCGGTCGGGA
CCGAGCGGGTCTCTCCACGGCAACCGCCGACGTCACGAACGTACAACTG
TACCGTCGCGAGAGGACGTGATGCGCCCGGTGATTGGCGCCGCCGCTGC
GGCTGCGCAGGAGACGACCCCCGCGGGCGCTCCCACCCCCATCTCGCGC
GGACTCGCTTTA

The sequence (5′-3′) after the bisulfite treatment of the target sequence of the TSPYL5 gene is as follows:

SEQ ID NO: 30
CGGATTCGGGTTTTGGCGCGGTTTTTGTTTCGGTTTTTGGCGCGGGAGG
ATTTTCGATTTCGATTTCGGTCGTTTATGGTGGCGGCGGAGGTAGTTTT
AAAGATACGTTGTGATTTTGCGGTTTTTGACGTTAGTTTTCGGTCGGGA
TCGAGCGGGTTTTTTTACGGTAATCGTCGACGTTACGAACGTATAATTG
TATCGTCGCGAGAGGACGTGATGCGTTCGGTGATTGGCGTCGTCGTTGC
GGTTGCGTAGGAGACGATTTTCGCGGGCGTTTTTATTTTTATTTCGCGC
GGATTCGTTTTA

The complementary sequence (5′-3′) of the target sequence of the TSPYL5 gene is as follows:

SEQ ID NO: 31
TAAAGCGAGTCCGCGCGAGATGGGGGTGGGAGCGCCCGCGGGGGTCGTC
TCCTGCGCAGCCGCAGCGGCGGCGCCAATCACCGGGCGCATCACGTCCT
CTCGCGACGGTACAGTTGTACGTTCGTGACGTCGGCGGTTGCCGTGGAG
AGACCCGCTCGGTCCCGACCGAGAGCTGGCGTCAGGAGCCGCAGGGTCA
CAGCGTGTCTTTGAAGCTGCCTCCGCCGCCACCATGAGCGGCCGAAGTC
GGGGTCGAAAGTCCTCCCGCGCCAAAAACCGGGGCAAAGGCCGCGCCAA
AGCCCGAGTCCG

The sequence (5′-3′) after the bisulfite treatment of the complementary sequence of the target sequence of the TSPYL5 gene is as follows:

SEQ ID NO: 32
TAAAGCGAGTTCGCGCGAGATGGGGGTGGGAGCGTTCGCGGGGGTCGTT
TTTTGCGTAGTCGTAGCGGCGGCGTTAATTATCGGGCGTATTACGTTTT
TTCGCGACGGTATAGTTGTACGTTCGTGACGTCGGCGGTTGTCGTGGAG
AGATTCGTTCGGTTTCGATCGAGAGTTGGCGTTAGGAGTCGTAGGGTTA
TAGCGTGTTTTTGAAGTTGTTTTCGTCGTTATTATGAGCGGTCGAAGTC
GGGGTCGAAAGTTTTTTCGCGTTAAAAATCGGGGTAAAGGTCGCGTTAA
AGTTCGAGTTCG

The target sequence (5′-3′) of the EMX1 gene is as follows:

SEQ ID NO: 33
CCGAGGGCCGGGGGCGCCTGGAGAGAAATCCAGCTCCGGCTCTGAGCGT
CTCCAGTCAGGCGAGGCGGATAAATCCTTCGCAAAACCCTCTTGGAAAT
TGCCGCCGCTTCCTGAGCCATCAGTCCCAGCGGGTACGTTATCGAGTAG
CACAAACAGTTGGATTTTTCCCTCAAGAACCGAGTCTGGACGCGGAGAT
GGAGCCAAGTGTGGCTGCATTTTCGGACCCGGAAATCCGTTGGGCACTG
AAGGACTTTTCGAACCCTGTAGCGCTGTTGCTTCGCGGTCCATCGTCGC
CGCTGCAGACGGATGCGCTCCCCGGCGGCTCTACGCCCTCCAGTCCCGG
CCAGGCCTCTGGGCTGGGAGCCGAGCCGTCTCGGGCCCTCCGGCGCCGC
GTTTTCTA

The sequence (5′-3′) after the bisulfite treatment of the target sequence of the EMX1 gene is as follows:

SEQ ID NO: 34
TCGAGGGTCGGGGGCGTTTGGAGAGAAATTTAGTTTCGGTTTTGAGCGT
TTTTAGTTAGGCGAGGCGGATAAATTTTTCGTAAAATTTTTTTGGAAAT
TGTCGTCGTTTTTTGAGTTATTAGTTTTAGCGGGTACGTTATCGAGTAG
TATAAATAGTTGGATTTTTTTTTTAAGAATCGAGTTTGGACGCGGAGAT
GGAGTTAAGTGTGGTTGTATTTTCGGATTCGGAAATTCGTTGGGTATTG
AAGGATTTTTCGAATTTTGTAGCGTTGTTGTTTCGCGGTTTATCGTCGT
CGTTGTAGACGGATGCGTTTTTCGGCGGTTTTACGTTTTTTAGTTTCGG
TTAGGTTTTTGGGTTGGGAGTCGAGTCGTTTCGGGTTTTTCGGCGTCGC
GTTTTTTA

The complementary sequence (5′-3′) of the target sequence of the EMX1 gene is as follows:

SEQ ID NO: 35
TAGAAAACGCGGCGCCGGAGGGCCCGAGACGGCTCGGCTCCCAGCCCAG
AGGCCTGGCCGGGACTGGAGGGCGTAGAGCCGCCGGGGAGCGCATCCGT
CTGCAGCGGCGACGATGGACCGCGAAGCAACAGCGCTACAGGGTTCGAA
AAGTCCTTCAGTGCCCAACGGATTTCCGGGTCCGAAAATGCAGCCACAC
TTGGCTCCATCTCCGCGTCCAGACTCGGTTCTTGAGGGAAAAATCCAAC
TGTTTGTGCTACTCGATAACGTACCCGCTGGGACTGATGGCTCAGGAAG
CGGCGGCAATTTCCAAGAGGGTTTTGCGAAGGATTTATCCGCCTCGCCT
GACTGGAGACGCTCAGAGCCGGAGCTGGATTTCTCTCCAGGCGCCCCCG
GCCCTCGG

The sequence (5′-3′) after the bisulfite treatment of the complementary sequence of the target sequence of the EMX1 gene is as follows:

SEQ ID NO: 36
TAGAAAACGCGGCGTCGGAGGGTTCGAGACGGTTCGGTTTTTAGTTTAG
AGGTTTGGTCGGGATTGGAGGGCGTAGAGTCGTCGGGGAGCGTATTCGT
TTGTAGCGGCGACGATGGATCGCGAAGTAATAGCGTTATAGGGTTCGAA
AAGTTTTTTAGTGTTTAACGGATTTTCGGGTTCGAAAATGTAGTTATAT
TTGGTTTTATTTTCGCGTTTAGATTCGGTTTTTGAGGGAAAAATTTAAT
TGTTTGTGTTATTCGATAACGTATTCGTTGGGATTGATGGTTTAGGAAG
CGGCGGTAATTTTTAAGAGGGTTTTGCGAAGGATTTATTCGTTTCGTTT
GATTGGAGACGTTTAGAGTCGGAGTTGGATTTTTTTTTAGGCGTTTTCG
GTTTTCGG

The target sequences and related sequences of CHFR gene, SPINT2 gene, RNF135 gene, CHST2 gene, SPDYA gene, PAX5 gene, VASH2 gene, TSPYL5 gene and EMX1 gene are shown in Table 1:

TABLE 1
Target sequences and related sequences of
RNF135 gene, CHFR gene and PAX5 gene.

| | Sequence |
|---|---|
| Name of target sequence | number |
| The target sequence of the RNF135 gene | SEQ ID NO: 1 |
| The sequence obtained after bisulfite | SEQ ID NO: 2 |
| treatment of the target sequence of the RNF135 gene | |
| The complementary sequence of the target | SEQ ID NO: 3 |
| sequence of the RNF135 gene | |
| The sequence obtained after bisulfite treatment | SEQ ID NO: 4 |
| of the complementary sequence of the target | |
| sequence of the RNF135 gene | |
| The target sequence of the CHFR gene | SEQ ID NO: 5 |
| The sequence obtained after bisulfite treatment | SEQ ID NO: 6 |
| of the target sequence of the CHFR gene | |
| The complementary sequence of the target | SEQ ID NO: 7 |
| sequence of the CHFR gene | |
| The sequence obtained after bisulfite treatment | SEQ ID NO: 8 |
| of the complementary sequence of the target | |
| sequence of the CHFR gene | |
| The target sequence of the PAX5 gene | SEQ ID NO: 9 |
| The sequence obtained after bisulfite treatment | SEQ ID NO: 10 |
| of the target sequence of the PAX5 gene | |
| The complementary sequence of the target sequence | SEQ ID NO: 11 |
| of the PAX5 gene | |
| The sequence obtained after bisulfite treatment | SEQ ID NO: 12 |
| of the complementary sequence of the target | |
| sequence of the PAX5 gene | |
| The target sequence of the SPINT2 gene | SEQ ID NO: 13 |
| The sequence obtained after bisulfite treatment | SEQ ID NO: 14 |
| of the target sequence of the SPINT2 gene | |
| The complementary sequence of the target sequence | SEQ ID NO: 15 |
| of the SPINT2 gene | |
| The sequence obtained after bisulfite treatment | SEQ ID NO: 16 |
| of the complementary sequence of the target sequence | |
| of the SPINT2 gene | |
| The target sequence of the SPDYA gene | SEQ ID NO: 17 |
| The sequence obtained after bisulfite treatment | SEQ ID NO: 18 |
| of the target sequence of the SPDYA gene | |
| The complementary sequence of the target sequence | SEQ ID NO: 19 |
| of the SPDYA gene | |
| The sequence obtained after bisulfite treatment | SEQ ID NO: 20 |
| of the complementary sequence of the target | |
| sequence of the SPDYA gene | |
| The target sequence of the VASH2 gene | SEQ ID NO: 21 |
| The sequence obtained after bisulfite treatment | SEQ ID NO: 22 |
| of the target sequence of the VASH2 gene | |
| The complementary sequence of the target sequence | SEQ ID NO: 23 |
| of the VASH2 gene | |
| The sequence obtained after bisulfite treatment | SEQ ID NO: 24 |
| of the complementary sequence of the target | |
| sequence of the VASH2 gene | |
| The target sequence of the CHST2 gene | SEQ ID NO: 25 |
| The sequence obtained after bisulfite treatment | SEQ ID NO: 26 |
| of the target sequence of the CHST2 gene | |
| The complementary sequence of the target sequence | SEQ ID NO: 27 |
| of the CHST2 gene | |
| The sequence obtained after bisulfite treatment | SEQ ID NO: 28 |
| of the complementary sequence of the target | |
| sequence of the CHST2 gene | |
| The target sequence of the TSPYL5 gene | SEQ ID NO: 29 |
| The sequence obtained after bisulfite treatment | SEQ ID NO: 30 |
| of the target sequence of the TSPYL5 gene | |
| The complementary sequence of the target sequence | SEQ ID NO: 31 |
| of the TSPYL5 gene | |
| The sequence obtained after bisulfite treatment | SEQ ID NO: 32 |
| of the complementary sequence of the target | |
| sequence of the TSPYL5 gene | |
| The target sequence of the EMX1 gene | SEQ ID NO: 33 |
| The sequence obtained after bisulfite treatment | SEQ ID NO: 34 |
| of the target sequence of the EMX1 gene | |
| The complementary sequence of the target sequence | SEQ ID NO: 35 |
| of the EMX1 gene | |
| The sequence obtained after bisulfite treatment | SEQ ID NO: 36 |
| of the complementary sequence of the target | |
| sequence of the EMX1 gene | |

Those skilled in the art will also understand that the target sequences of the CHFR gene, SPINT2 gene, RNF135 gene, CHST2 gene, SPDYA gene, PAX5 gene, VASH2 gene, TSPYL5 gene and EMX1 gene are not limited to the specific sequences listed above. The target sequence of the CHFR gene should encompass: a sequence comprising one, two, or three or more nucleotide mutations compared to the sequence shown as any one of SEQ ID NOs: 1-4, but still having substantially the same essential function as the sequence shown as any one of SEQ ID NOs: 1-4; a sequence having 95%, 96%, 97%, 98% or 99% sequence identity to the sequence shown as any one of SEQ ID NOs: 1-4; and a sequence in which one or more nucleotides are deleted, one or more nucleotides are added, or one or more nucleotides are substituted from any of the nucleotide sequences shown as SEQ ID NO: 1-4 but still having 90%, 91%, 92%, 93%, 94%, 95%, 96%, 97%, 98% or 99% identity to the nucleotide sequence shown as any one of SEQ ID NOs: 1-4. The target sequence of the SPINT2 gene should encompass: a sequence comprising one, two, or three or more nucleotide mutations compared to the sequence shown as any one of SEQ ID NOs: 5-8, but still having substantially the same essential function as the sequence shown as any one of SEQ ID NOs: 5-8; a sequence having 95%, 96%, 97%, 98% or 99% sequence identity to the sequence shown as any one of SEQ ID NOs: 5-8; and a sequence in which one or more nucleotides are deleted, one or more nucleotides are added, or one or more nucleotides are substituted from any of the nucleotide sequences shown as SEQ ID NO: 5-8 but still having 90%, 91%, 92%, 93%, 94%, 95%, 96%, 97%, 98% or 99% identity to the nucleotide sequence shown as any one of SEQ ID NOs: 5-8. The target sequence of the RNF135 gene should encompass: a sequence comprising one, two, or three or more nucleotide mutations compared to the sequence shown as any one of SEQ ID NOs: 9-12, but still having substantially the same essential function as the sequence shown as any one of SEQ ID NOs: 9-12; a sequence having 95%, 96%, 97%, 98% or 99% sequence identity to the sequence shown as any one of SEQ ID NOs: 9-12; and a sequence in which one or more nucleotides are deleted, one or more nucleotides are added, or one or more nucleotides are substituted from any of the nucleotide sequences shown as SEQ ID NO:9-12 but still having 90%, 91%, 92%, 93%, 94%, 95%, 96%, 97%, 98% or 99% identity to the nucleotide sequence shown as any one of SEQ ID NOs: 9-12. The target sequence of the CHST2 gene should encompass: a sequence comprising one, two, or three or more nucleotide mutations compared to the sequence shown as any one of SEQ ID NOs: 13-16, but still having substantially the same essential function as the sequence shown as any one of SEQ ID NOs: 13-16; a sequence having 95%, 96%, 97%, 98% or 99% sequence identity to the sequence shown as any one of SEQ ID NOs: 13-16; and a sequence in which one or more nucleotides are deleted, one or more nucleotides are added, or one or more nucleotides are substituted from any of the nucleotide sequences shown as SEQ ID NO:13-16 but still having 90%, 91%, 92%, 93%, 94%, 95%, 96%, 97%, 98% or 99% identity to the nucleotide sequence shown as any one of SEQ ID NOs: 13-16. The target sequence of the SPDYA gene should encompass: a sequence comprising one, two, or three or more nucleotide mutations compared to the sequence shown as any one of SEQ ID NOs: 17-20, but still having substantially the same essential function as the sequence shown as any one of SEQ ID NOS: 17-20; a sequence having 95%, 96%, 97%, 98% or 99% sequence identity to the sequence shown as any one of SEQ ID NOs: 17-20; and a sequence in which one or more nucleotides are deleted, one or more nucleotides are added, or one or more nucleotides are substituted from any of the nucleotide sequences shown as SEQ ID NO: 17-20 but still having 90%, 91%, 92%, 93%, 94%, 95%, 96%, 97%, 98% or 99% identity to the nucleotide sequence shown as any one of SEQ ID NOs: 17-20. The target sequence of the PAX5 gene should encompass: a sequence comprising one, two, or three or more nucleotide mutations compared to the sequence shown as any one of SEQ ID NOs: 21-24, but still having substantially the same essential function as the sequence shown as any one of SEQ ID NOs: 21-24; a sequence having 95%, 96%, 97%, 98% or 99% sequence identity to the sequence shown as any one of SEQ ID NOs: 21-24; and a sequence in which one or more nucleotides are deleted, one or more nucleotides are added, or one or more nucleotides are substituted from any of the nucleotide sequences shown as SEQ ID NO:21-24 but still having 90%, 91%, 92%, 93%, 94%, 95%, 96%, 97%, 98% or 99% identity to the nucleotide sequence shown as any one of SEQ ID NOs: 21-24. The target sequence of the VASH2 gene should encompass: a sequence comprising one, two, or three or more nucleotide mutations compared to the sequence shown as any one of SEQ ID NOs: 25-28, but still having substantially the same essential function as the sequence shown as any one of SEQ ID NOs: 25-28; a sequence having 95%, 96%, 97%, 98% or 99% sequence identity to the sequence shown as any one of SEQ ID NOs: 25-28; and a sequence in which one or more nucleotides are deleted, one or more nucleotides are added, or one or more nucleotides are substituted from any of the nucleotide sequences shown as SEQ ID NO:25-28 but still having 90%, 91%, 92%, 93%, 94%, 95%, 96%, 97%, 98% or 99% identity to the nucleotide sequence shown as any one of SEQ ID NOs: 25-28. The target sequence of the TSPYL5 gene should encompass: a sequence comprising one, two, or three or more nucleotide mutations compared to the sequence shown as any one of SEQ ID NOs: 29-32, but still having substantially the same essential function as the sequence shown as any one of SEQ ID NOS: 29-32; a sequence having 95%, 96%, 97%, 98% or 99% sequence identity to the sequence shown as any one of SEQ ID NOs: 29-32; and a sequence in which one or more nucleotides are deleted, one or more nucleotides are added, or one or more nucleotides are substituted from any of the nucleotide sequences shown as SEQ ID NO:29-32 but still having 90%, 91%, 92%, 93%, 94%, 95%, 96%, 97%, 98% or 99% identity to the nucleotide sequence shown as any one of SEQ ID NOs: 29-32. The target sequence of the EMX1 gene should encompass: a sequence comprising one, two, or three or more nucleotide mutations compared to the sequence shown as any one of SEQ ID NOs: 33-36, but still having substantially the same essential function as the sequence shown as any one of SEQ ID NOs: 33-36; a sequence having 95%, 96%, 97%, 98% or 99% sequence identity to the sequence shown as any one of SEQ ID NOs: 33-36; and a sequence in which one or more nucleotides are deleted, one or more nucleotides are added, or one or more nucleotides are substituted from any of the nucleotide sequences shown as SEQ ID NO:33-36 but still having 90%, 91%, 92%, 93%, 94%, 95%, 96%, 97%, 98% or 99% identity to the nucleotide sequence shown as any one of SEQ ID NOs: 33-36.

Preferably, the nucleic acid used to detect the methylation status of the target gene comprises a fragment of at least 9 nucleotides in the target sequence of the target gene, wherein the fragment comprises at least one CpG dinucleotide sequence. In certain preferred embodiments, if bisulfite is used to convert the DNA of the sample to be tested, the nucleic acid used to detect the methylation status of the target gene includes a fragment of at least 9 nucleotides in the sequence obtained after bisulfite conversion of the target sequence of the target gene, preferably a fragment of at least 10, 11, 12, 13, 14, 15, 16, 17, 18, 19, 20, 21, 22 or more nucleotides, wherein the nucleotide fragment comprises at least one CpG dinucleotide sequence.

More preferably, the nucleic acid used to detect the methylation status of the target gene includes a fragment that hybridizes to at least 15 nucleotides of the target sequence of the target gene under moderately stringent or stringent conditions, wherein the nucleotide fragment comprises at least one CpG dinucleotide sequence. In certain preferred embodiments, if bisulfite is used to convert the DNA of the sample to be tested, the nucleic acid used to detect the methylation status of the target gene includes a fragment that hybridizes to at least 15 nucleotides (preferably at least 16, 17, 18, 19, 20, 21, 22 or more nucleotides) in the sequence obtained after bisulfite conversion of the target sequence of the target gene under moderately stringent or stringent conditions, wherein the nucleotide fragment comprises at least one CpG dinucleotide sequence.

Preferably, the composition further comprises a reagent for converting the 5-unmethylated cytosine base of the target sequence of the target gene to uracil. More preferably, the reagent is bisulfite.

The nucleic acid used to detect the methylation status of the target gene may further include a blocker that preferentially binds to DNA in its unmethylated state.

Preferably, the composition comprises one or more of the primers and probes as shown in Table 2:

TABLE 2
Sequences of primer and probe used in the present application.

| Sequence | Sequence | Specific nucleotide sequence |
|---|---|---|
| number | name | (5′-3′) |
| SEQ ID NO: 37 | CHFR_1F | GGGTGGAATTTAGTAGCG |
| SEQ ID NO: 38 | CHFR_1R | CTCTAAAATTTAACTCCCAAATAATACG |
| SEQ ID NO: 39 | CHFR_1P | TTGAGTCGTCGCGAAGTGTCGG |
| SEQ ID NO: 40 | SPINT2_1F | TATTGGTTTTGGCGATTTTC |
| SEQ ID NO: 41 | SPINT2_1R | ACTCAACGACCCTCAACG |
| SEQ ID NO: 42 | SPINT2_1P | AGGTGTAGCGCGGTTTTGAAC |
| SEQ ID NO: 43 | RNF135_1F | TAGGGGTTGTTGGATTGGTTC |
| SEQ ID NO: 44 | RNF135_1R | CACAAAACCTCCAAACAATAACG |
| SEQ ID NO: 45 | RNF135_1P | TACGTTGTTTTGCGGTTATAGTTTTTGTC |
| SEQ ID NO: 46 | CHST2_1F | TAAGATGTTTTTAGATACGTCGC |
| SEQ ID NO: 47 | CHST2_1R | GCCTACCCTTTCCTCG |
| SEQ ID NO: 48 | CHST2_1P | CGACAATATAAAACGACGCGCTA |
| SEQ ID NO: 49 | SPDYA_1F | TTTTAAGTTTATTAGTTTTTAGCGT |
| SEQ ID NO: 50 | SPDYA_1R | TTCTCGCGAAAACAAACG |
| SEQ ID NO: 51 | SPDYA_1P | AAACGAAAACGACCCGAAACGA |
| SEQ ID NO: 52 | PAX5_1F | AGATACGTCGCGTTTTTAGC |
| SEQ ID NO: 53 | PAX5_1R | CGCCTACCCTTTCCTCG |
| SEQ ID NO: 54 | PAX5_1P | TCGTTTTATATTGTCGGTTCGGAGC |
| SEQ ID NO: 55 | VASH2_1F | GCGGGGTTTTTAGGATATTATAGC |
| SEQ ID NO: 56 | VASH2_1R | AAAAAACAAAAATAAAAAAAACCTAACG |
| SEQ ID NO: 57 | VASH2_1P | GCGATCGGATTTCGCGTGGT |
| SEQ ID NO: 58 | TSPYL5_1F | CGTTAGTTTTCGGTCGG |
| SEQ ID NO: 59 | TSPYL5_1R | CGACGATACAATTATACGTT |
| SEQ ID NO: 60 | TSPYL5_1P | ACGGTAATCGTCGACGTT |
| SEQ ID NO: 61 | EMX1_1F | TTCGAATTTTGTAGCGTTG |
| SEQ ID NO: 62 | EMX1_1R | GCCGAAAAACGCATCC |
| SEQ ID NO: 63 | EMX1_1P | CTACAACGACGACGATAAACC |

In Table 2, “F” represents a forward primer; “R” represents a reverse primer; and “P” represents a probe.

Preferably, the fluorescent labeling method of the probe sequence used herein is as shown in Table 3.

TABLE 3
A fluorescent labeling method for the probe
sequences used in the present application.

| Sequence changes | Sequence name | 5′ label | 3′ label |
|---|---|---|---|
| SEQ ID NO: 39 | CHFR_1P | FAM | BHQ1 |
| SEQ ID NO: 42 | SPINT2_1P | FAM | BHQ1 |
| SEQ ID NO: 45 | RNF135_1P | FAM | BHQ1 |
| SEQ ID NO: 48 | CHST2_1P | FAM | BHQ1 |
| SEQ ID NO: 51 | SPDYA_1P | FAM | BHQ1 |
| SEQ ID NO: 54 | PAX5_1P | FAM | BHQ1 |
| SEQ ID NO: 57 | VASH2_1P | FAM | BHQ1 |
| SEQ ID NO: 60 | TSPYL5_1P | FAM | BHQ1 |
| SEQ ID NO: 63 | EMX1_1P | FAM | BHQ1 |

In certain embodiments, the composition further comprises an agent that converts the 5-unmethylated cytosine base of the gene to uracil. Preferably, the reagent is bisulfite. Bisulfite modification of DNA is a known tool for assessing CpG methylation status. 5-Methylcytosine is the most common covalent base modification in eukaryotic DNA. 5-Methylcytosine cannot be identified by sequencing because 5-methylcytosine has the same base pairing behavior as cytosine. In addition, the epigenetic information carried by 5-methylcytosine is completely lost during PCR amplification. The method most commonly used to analyze the presence of 5-methylcytosine in DNA is based on the specific reaction of bisulfite with cytosine; after subsequent alkaline hydrolysis, unmethylated cytosine is converted to uracil, which corresponds to thymine in terms of pairing behavior; however, under these conditions, 5-methylcytosine remains unmodified. The original DNA is thus converted in such a way that the 5-methylcytosine, which was previously indistinguishable from cytosine in terms of its hybridization behavior, can now be detected as the only remaining cytosine by conventional, known molecular biology techniques, for example by amplification and hybridization. All of these technologies are based on different base-pairing properties that can now be fully made use of. Thus, typically, the present application provides for the use of bisulfite technology in combination with one or more methylation assays to determine the methylation status of CpG dinucleotide sequences within the target sequence of the target gene. In addition, the method of the present application is suitable for analyzing heterogeneous samples, e.g., low-concentration tumor cells in blood or feces. Therefore, when analyzing the methylation status of CpG dinucleotide sequences in such samples, one skilled in the art can use quantitative assays to determine the methylation level (e.g., percentage, fraction, ratio, proportion, or degree) of a particular CpG dinucleotide sequence, rather than the methylation status. Accordingly, the term methylation status or methylation state should also be considered to refer to a value reflecting the methylation status of a CpG dinucleotide sequence.

In another aspect, the present application provides an oligonucleotide for detecting liver cancer in vitro, comprising: a fragment of at least 9 nucleotides of SEQ ID NO: 1 or SEQ ID NO: 2 or SEQ ID NO: 3 or SEQ ID NO: 4 or a complementary sequence thereof and comprising at least one CpG dinucleotide sequence; and/or a fragment of at least 9 nucleotides of SEQ ID NO: 5 or SEQ ID NO: 6 or SEQ ID NO: 7 or SEQ ID NO: 8 a complementary sequence thereof and comprising at least one CpG dinucleotide sequence; and/or a fragment of at least 9 nucleotides of SEQ ID NO: 9 or SEQ ID NO: 10 or SEQ ID NO: 11 or SEQ ID NO: 12 or a complementary sequence thereof and comprising at least one CpG dinucleotide sequence; and/or a fragment of at least 9 nucleotides of SEQ ID NO: 13 or SEQ ID NO: 14 or SEQ ID NO: 15 or SEQ ID NO: 16 or a complementary sequence thereof and comprising at least one CpG dinucleotide sequence; and/or a fragment of at least 9 nucleotides of SEQ ID NO: 17 or SEQ ID NO: 18 or SEQ ID NO: 19 or SEQ ID NO: NO: 20 or a complementary sequence thereof and comprising at least one CpG dinucleotide sequence; and/or a fragment of at least 9 nucleotides of SEQ ID NO: 21 or SEQ ID NO: 22 or SEQ ID NO: 23 or SEQ ID NO: 24 or a complementary sequence thereof and comprising at least one CpG dinucleotide sequence; and/or a fragment of at least 9 nucleotides of SEQ ID NO: 25 or SEQ ID NO: 26 or SEQ ID NO: 27 or SEQ ID NO: 28 or a complementary sequence thereof and comprising at least one CpG dinucleotide sequence; and/or a fragment of at least 9 nucleotides of SEQ ID NO: 29 or SEQ ID NO: 30 or SEQ ID NO: 31 or SEQ ID NO: 32 or a complementary sequence thereof and comprising at least one CpG dinucleotide sequence; and/or a fragment of at least 9 nucleotides of SEQ ID NO: 33 or SEQ ID NO: 34 or SEQ ID NO: 35 or SEQ ID NO: 36 or a complementary sequence thereof and comprising at least one CpG dinucleotide sequence.

Preferably, the oligonucleotide for detecting liver cancer in vitro comprises: a fragment of at least 9 nucleotides in a sequence obtained after bisulfite conversion of SEQ ID NO: 1 or SEQ ID NO: 2 or SEQ ID NO: 3 or SEQ ID NO: 4 or a complementary sequence thereof; and/or a fragment of at least 9 nucleotides in a sequence obtained after bisulfite conversion of SEQ ID NO: 5 or SEQ ID NO: 6 or SEQ ID NO: 7 or SEQ ID NO: 8 or a complementary sequence thereof and comprising at least one CpG dinucleotide sequence; and/or a fragment of at least 9 nucleotides in a sequence obtained after bisulfite conversion of SEQ ID NO: 9 or SEQ ID NO: 10 or SEQ ID NO: 11 or SEQ ID NO: 12 or a complementary sequence thereof and comprising at least one CpG dinucleotide sequence; a fragment of at least 9 nucleotides in a sequence obtained after bisulfite conversion of SEQ ID NO: 13 or SEQ ID NO: 14 or SEQ ID NO: 15 or SEQ ID NO: 16 or a complementary sequence thereof; and/or a fragment of at least 9 nucleotides in a sequence obtained after bisulfite conversion of SEQ ID NO: 17 or SEQ ID NO: 18 or SEQ ID NO: 19 or SEQ ID NO: NO: 20 or a complementary sequence thereof and comprising at least one CpG dinucleotide sequence; and/or a fragment of at least 9 nucleotides in a sequence obtained after bisulfite conversion of SEQ ID NO:21 or SEQ ID NO:22 or SEQ ID NO:23 or SEQ ID NO:24 or a complementary sequence thereof and comprising at least one CpG dinucleotide sequence; and/or a fragment of at least 9 nucleotides in a sequence obtained after bisulfite conversion of SEQ ID NO:25 or SEQ ID NO:26 or SEQ ID NO:27 or SEQ ID NO:28 or a complementary sequence thereof; and/or a fragment of at least 9 nucleotides in a sequence obtained after bisulfite conversion of SEQ ID NO:29 or SEQ ID NO:30 or SEQ ID NO:31 or SEQ ID NO:32 or a complementary sequence thereof and comprising at least one CpG dinucleotide sequence; and/or a fragment of at least 9 nucleotides in a sequence obtained after bisulfite conversion of SEQ ID NO: 33 or SEQ ID NO:34 or SEQ ID NO:35 or SEQ ID NO:36 or a complementary sequence thereof and comprising at least one CpG dinucleotide sequence.

The oligonucleotides for detecting liver cancer in vitro of the present application also include: a fragment that hybridizes to at least 15 nucleotides in SEQ ID NO: 1 or SEQ ID NO: 2 or SEQ ID NO: 3 or SEQ ID NO: 4 or a complementary sequence thereof under moderate stringency or stringent conditions and comprises at least one CpG dinucleotide sequence; and/or a fragment that hybridizes to at least 15 nucleotides in SEQ ID NO: 5 or SEQ ID NO: 6 or SEQ ID NO: 7 or SEQ ID NO: 8 or a complementary sequence thereof under moderate stringency or stringent conditions and comprises at least one CpG dinucleotide sequence; and/or a fragment that hybridizes to at least 15 nucleotides in SEQ ID NO: 9 or SEQ ID NO: 10 or SEQ ID NO: 11 or SEQ ID NO: 12 or a complementary sequence thereof under moderate stringency or stringent conditions and comprises at least one CpG dinucleotide sequence; and/or a fragment that hybridizes to at least 15 nucleotides in SEQ ID NO: 13 or SEQ ID NO: 14 or SEQ ID NO: 15 or SEQ ID NO: 16 or a complementary sequence thereof under moderate stringency or stringent conditions and comprises at least one CpG dinucleotide sequence; and/or a fragment that hybridizes to at least 15 nucleotides in SEQ ID NO: 17 or SEQ ID NO: 18 or SEQ ID NO: 19 or SEQ ID NO: 20 or a complementary sequence thereof under moderately stringent or stringent conditions and comprises at least one CpG dinucleotide sequence; and/or a fragment that hybridizes to at least 15 nucleotides in SEQ ID NO: 21 or SEQ ID NO: 22 or SEQ ID NO: 23 or SEQ ID NO: 24 or a complementary sequence thereof under moderately stringent or stringent conditions and comprises at least one CpG dinucleotide sequence; and/or a fragment that hybridizes to at least 15 nucleotides in SEQ ID NO: 25 or SEQ ID NO: 26 or SEQ ID NO: 27 or SEQ ID NO: 28 or a complementary sequence thereof under moderately stringent or stringent conditions and comprises at least one CpG dinucleotide sequence; and/or a fragment that hybridizes to at least 15 nucleotides in SEQ ID NO: 29 or SEQ ID NO: 30 or SEQ ID NO: 31 or SEQ ID NO: NO: 32 or a complementary sequence thereof under moderately stringent or stringent conditions and comprises at least one CpG dinucleotide sequence, and/or a fragment that hybridizes to at least 15 nucleotides in SEQ ID NO: 33 or SEQ ID NO: 34 or SEQ ID NO: 35 or SEQ ID NO: 36 or a complementary sequence thereof under moderately stringent or stringent conditions and comprises at least one CpG dinucleotide sequence.

Preferably, the oligonucleotide for detecting liver cancer in vitro comprises: a fragment that hybridizes to at least 15 nucleotides in a sequence obtained after bisulfite conversion of SEQ ID NO: 1 or SEQ ID NO: 2 or SEQ ID NO: 3 or SEQ ID NO: 4 or a complementary sequence thereof under moderate stringency or stringent conditions and comprises at least one CpG dinucleotide sequence; and/or a fragment that hybridizes to at least 15 nucleotides in a sequence obtained after bisulfite conversion of SEQ ID NO: 5 or SEQ ID NO: 6 or SEQ ID NO: 7 or SEQ ID NO: 8 or a complementary sequence thereof under moderate stringency or stringent conditions and comprises at least one CpG dinucleotide sequence; and/or a fragment that hybridizes to at least 15 nucleotides in a sequence obtained after bisulfite conversion of SEQ ID NO: 9 or SEQ ID NO: 10 or SEQ ID NO: 11 or SEQ ID NO: 12 or a complementary sequence thereof under moderate stringency or stringent conditions and comprises at least one CpG dinucleotide sequence; and/or a fragment that hybridizes to at least 15 nucleotides in a sequence obtained after bisulfite conversion of SEQ ID NO: 13 or SEQ ID NO: 14 or SEQ ID NO: 15 or SEQ ID NO: 16 or a complementary sequence thereof under moderate stringency or stringent conditions and comprises at least one CpG dinucleotide sequence; and/or a fragment that hybridizes to at least 15 nucleotides in a sequence obtained after bisulfite conversion of SEQ ID NO: 17 or SEQ ID NO: 18 or SEQ ID NO: 19 or SEQ ID NO: 20 or a complementary sequence thereof under moderate stringency or stringent conditions and comprises at least one CpG dinucleotide sequence; and/or a fragment that hybridizes to at least 15 nucleotides in a sequence obtained after bisulfite conversion of SEQ ID NO: 21 or SEQ ID NO: 22 or SEQ ID NO: 23 or SEQ ID NO: 24 or a complementary sequence thereof under moderate stringency or stringent conditions and comprises at least one CpG dinucleotide sequence; and/or a fragment that hybridizes to at least 15 nucleotides in a sequence obtained after bisulfite conversion of SEQ ID NO: 25 or SEQ ID NO: 26 or SEQ ID NO: 27 or SEQ ID NO: 28 or a complementary sequence thereof under moderate stringency or stringent conditions and comprises at least one CpG dinucleotide sequence; and/or a fragment that hybridizes to at least 15 nucleotides in a sequence obtained after bisulfite conversion of SEQ ID NO: 29 or SEQ ID NO: 30 or SEQ ID NO: 31 or SEQ ID NO: 32 or a complementary sequence thereof under moderate stringency or stringent conditions and comprises at least one CpG dinucleotide sequence; and/or a fragment that hybridizes to at least 15 nucleotides in a sequence obtained after bisulfite conversion of SEQ ID NO: 33 or SEQ ID NO: 34 or SEQ ID NO: 35 or SEQ ID NO: 36 or a complementary sequence thereof under moderate stringency or stringent conditions and comprises at least one CpG dinucleotide sequence.

The oligonucleotide for detecting liver cancer in vitro of the present application may further comprise: a blocker that preferentially binds to DNA in its unmethylated state.

In a specific embodiment, the oligonucleotide for detecting liver cancer in vitro comprises the sequences of SEQ ID NO: 37 and SEQ ID NO: 38, and further comprises the sequence of SEQ ID NO: 39.

In another specific embodiment, the oligonucleotide for detecting liver cancer in vitro comprises the sequences of SEQ ID NO: 40 and SEQ ID NO: 41, and further comprises the sequence of SEQ ID NO: 42.

In another specific embodiment, the oligonucleotide for detecting liver cancer in vitro comprises the sequences of SEQ ID NO: 43 and SEQ ID NO: 44, and further comprises the sequence of SEQ ID NO: 45.

In another specific embodiment, the oligonucleotide for detecting liver cancer in vitro comprises the sequences of SEQ ID NO: 46 and SEQ ID NO: 47, and further comprises the sequence of SEQ ID NO: 48.

In another specific embodiment, the oligonucleotide for detecting liver cancer in vitro comprises the sequences of SEQ ID NO: 49 and SEQ ID NO: 50, and further comprises the sequence of SEQ ID NO: 51.

In another specific embodiment, the oligonucleotide for detecting liver cancer in vitro comprises the sequences of SEQ ID NO: 52 and SEQ ID NO: 53, and further comprises the sequence of SEQ ID NO: 54.

In another specific embodiment, the oligonucleotide for detecting liver cancer in vitro comprises the sequences of SEQ ID NO: 55 and SEQ ID NO: 56, and further comprises the sequence of SEQ ID NO: 57.

In another specific embodiment, the oligonucleotide for detecting liver cancer in vitro comprises the sequences of SEQ ID NO: 58 and SEQ ID NO: 59, and further comprises the sequence of SEQ ID NO: 60.

In another specific embodiment, the oligonucleotide for detecting liver cancer in vitro comprises the sequences of SEQ ID NO: 61 and SEQ ID NO: 62, and further comprises the sequence of SEQ ID NO: 63.

In another aspect, the present application provides a kit comprising the composition. The kit further comprises at least one other component selected from the group consisting of: a nucleoside triphosphate, a DNA polymerase, and a buffer required for the function of the DNA polymerase.

Typically, the kit also includes a container for holding the sample from patient. Furthermore, the kit also includes instructions for use and interpretation of the testing results.

The present application also relates to the use of the above-described composition and oligonucleotide in preparing a kit for detecting liver cancer in vitro.

In another aspect, the present application provides a method for detecting liver cancer in vitro, the method comprising the following steps:
1) isolating the target sequence of the target gene in the sample to be tested;
2) determining the methylation status of the target sequence of the target gene;
3) determining the status of the sample by the detection result of the methylation status of the target sequence of the target gene, thereby realizing the in vitro detection of liver cancer.

According to certain preferred embodiments, the method further comprises the following steps:
1) extracting genomic DNA from the sample to be tested;
2) treating the DNA sample obtained in step 1) with a reagent to convert the 5-unmethylated cytosine base to uracil or other bases, that is, converting the 5-unmethylated cytosine base of the target sequence of the target gene to uracil or other bases, wherein the converted base has a different hybridization property from the 5-unmethylated cytosine base and is detectable;
3) contacting the DNA sample treated in step 2) with a DNA polymerase and a primer for the target sequence of the target gene, so that the treated target sequence of the target gene is amplified to produce an amplified product or is not amplified; if the treated target sequence of the target gene undergoes a DNA polymerization reaction, an amplified product is produced; if the treated target sequence of the target gene does not undergo a DNA polymerization reaction, it is not amplified;
4) detecting the amplified product with a probe; and
5) determining the methylation status of at least one CpG dinucleotide of the target sequence of the target gene based on whether the amplified product is present or not.

Preferably, a typical primer includes a fragment of the target sequence of the target gene, and the fragment of the target sequence of the target gene comprises a fragment that is identical to, is complementary to, or hybridizes under moderately stringent or stringent conditions at least 9 nucleotides in any one of SEQ ID NOs: 1-4, any one of SEQ ID NOs: 5-8, any one of SEQ ID NOs: 9-12, any one of SEQ ID NOs: 13-16, any one of SEQ ID NOs: 17-20, any one of SEQ ID NOs: 21-24, any one of SEQ ID NOs: 25-28, any one of SEQ ID NOs: 29-32, or any one of SEQ ID NOs: 33-36, respectively.

Preferably, a typical probe includes a fragment of the target sequence of the target gene, and the fragment of the target sequence of the target gene comprises a fragment that is identical to, is complementary to, or hybridizes under moderately stringent or stringent conditions at least 15 nucleotides in any one of SEQ ID NOs: 1-4, any one of SEQ ID NOs: 5-8, any one of SEQ ID NOs: 9-12, any one of SEQ ID NOs: 13-16, any one of SEQ ID NOs: 17-20, any one of SEQ ID NOS: 21-24, any one of SEQ ID NOs: 25-28, any one of SEQ ID NOs: 29-32, or any one of SEQ ID NOS: 33-36, respectively.

Preferably, one or more of the primers and probes are as shown in Table 2 above.

Furthermore, the contacting or amplifying includes using at least one of the following methods: using a thermostable DNA polymerase as the amplification enzyme, using a polymerase lacking 5′-3′ exonuclease activity, using polymerase chain reaction (PCR), and generating amplification product nucleic acid molecules with a detectable label.

Preferably, the methylation status is determined by PCR, and the measurement methods such as “fluorescence-based real-time PCR technology”, methylation-sensitive single nucleotide primer extension reaction (Ms-SNuPE), methylation-specific PCR (MSP), and methylated CpG island amplification (MCA) are used to determine the methylation status of at least one CpG dinucleotide in the target sequence of the target gene. Among them, the “fluorescence-based real-time PCR” assay is a high-throughput quantitative methylation assay that uses fluorescence-based real-time PCR (TaqMan) technology and does not require further manipulation after the PCR step. Briefly, the “fluorescence-based real-time PCR” method begins with a mixed sample of genomic DNA, which is converted into a mixing pool of methylation-dependent sequence differences in a sodium bisulfite reaction according to standard operations. Fluorescence-based PCR was then performed in a “biased” reaction (employing PCR primers that overlap known CpG dinucleotides). Sequence differentiation can be generated at the level of amplification as well as at the level of fluorescence-detected amplification. “Fluorescence-based real-time PCR” assays can be used as quantitative tests of methylation status of genomic DNA samples, wherein sequence discrimination occurs at the level of probe hybridization. In this quantitative manner, a PCR reaction provides methylation-specific amplification in the presence of fluorescent probes that overlap specific CpG dinucleotides. A non-bias control for initial DNA amounts was provided by reactions in which neither the primer nor the probe covered any CpG dinucleotides. The “fluorescence-based real-time PCR” method can be used with any suitable probes, such as “TaqMan”, “Lightcycler”, etc. TaqMan probes are dual-labeled with a fluorescent reporter (RCHFRrter) and a quencher molecule (Quencher) and are designed to be specific for regions of relatively high GC content so that they melt at a temperature approximately 10° C. higher than that of the forward or reverse primer during PCR cycles. This allows the TaqMan probe to remain fully hybridized during the PCR annealing/extension step. When the Taq polymerase enzyme synthesizes a new strand in PCR, it eventually encounters the annealed TaqMan probe. The 5′ to 3′ endonuclease activity of Taq polymerase will then replace the TaqMan probe by digesting it, thereby releasing the fluorescent reporter molecule for quantitative detection of its now unquenched signal using a real-time fluorescence detection system. Typical reagents used for “fluorescence-based real-time PCR” analysis may include, but are not limited to: target sequence PCR primers for the target gene; non-specific amplification blockers; TaqMan or Lightcycler probes; optimized PCR buffer and deoxynucleotides; and Taq polymerase, etc.

In certain preferred embodiments, the methylation status of at least one CpG dinucleotide in the target sequence of the target gene is determined by a critical Ct value of a real-time PCR reaction. By using real-time PCR reactions to analyze DNA in biological samples, the methylation status of the target sequence of the target gene can be conveniently detected, and the critical Ct value of the PCR reaction can be used to rapidly and conveniently determine whether the tested sample is positive, thus providing a non-invasive and rapid in vitro detection method for liver cancer.

The sample is selected from: cell lines, histological sections, tissue biopsies/paraffin-embedded tissues, body fluids, feces, colon effluents, urine, plasma, serum, whole blood, isolated blood cells, cells isolated from blood, or a combination thereof. Preferred samples are plasma and tissues.

The present application also provides a system for detecting liver cancer, wherein the system comprises:
a data acquisition module for acquiring data on the methylation marker levels in subject samples; and
a calculation module of the probability of suffering from liver cancer for calculating the data obtained in the data acquisition module to calculate the probability of the subject suffering from liver cancer,
wherein the methylation marker(s) is/are one, two or three of an RNF135 gene, a CHFR gene and a PAX5 gene.

Furthermore, for the target sequences of the RNF135 gene, CHFR gene and PAX5 gene, the primers and probes used are as described above.

In a specific embodiment, the data acquisition module comprises the following submodules: an extraction module, for extracting genomic DNA from a sample;
a reagent treatment module, for treating the extracted genomic DNA with a bisulfite reagent to convert the 5-unmethylated cytosine base to uracil or other bases;
a detection module, for detecting the genomic DNA treated with a bisulfite reagent to obtain the methylation marker level of the sample.

In a specific embodiment, in the detection module, the genomic DNA treated with a bisulfite reagent is detected using real-time fluorescent quantitative PCR.

In a specific embodiment, in the calculation module for the probability of suffering from liver cancer, a pre-constructed formula for calculating the probability of a subject suffering from liver cancer based on data fitting of methylation marker levels is pre-stored.

In a specific embodiment, the formula is obtained by fitting data on the methylation level of RNF135 based on an existing database.

In a specific embodiment, the formula is obtained by fitting the data on the methylation level of RNF135 and the methylation level of PAX5 in an existing database.

In a specific embodiment, the formula is obtained by fitting data on the methylation level of RNF135, the methylation level of PAX5, and the methylation level of CHFR in an existing database.

In a specific embodiment, the formula is obtained by fitting the data on the methylation level RNF135, the methylation level of PAX5, and the methylation level of CHFR in the existing database using generalized linear regression. The generalized linear model is an extension of the linear model, which establishes the relationship between the mathematical expectation of the response variable and the linear combination of predictor variables through the link function. The main feature is that it does not forcibly change the natural measure of the data. It is a commonly used binary classification strategy.

In a specific embodiment, the formula is:

$$P = {e}^{( a * M 1 + b * M 2 + c * M 3 + 81 )} / ( {e}^{( a * M 1 + b * M 2 + c * M 3 + 81 )} + 1 ) ,$$

wherein P represents the probability of the subject suffering from liver cancer, M1 represents the Ct value of RNF135 determined by real-time fluorescence quantitative PCR, M2 represents the Ct value of PAX5 determined by real-time fluorescence quantitative PCR, and M3 represents the Ct value of CHFR determined by real-time fluorescence quantitative PCR.
a is selected from any value between 0.8 and 1.2, preferably 0.9;
b is selected from any value between 0.6 and 0.9, preferably 0.7;
c is selected from any value between 0.1 and 0.5, preferably 0.3.

In a specific embodiment, when the probability that the subject suffers from liver cancer, that is, P, is greater than or equal to 0.24, the subject is determined to have liver cancer.

The present application also provides a method for detecting liver cancer, wherein the method comprises:
a data acquisition step, for acquiring data on the methylation marker levels of a subject sample; and
a step of calculating the probability of suffering from liver cancer, for calculating the data obtained in the data acquisition step, thereby calculating the probability of the subject suffering from liver cancer,
wherein the methylation marker(s) is/are one, two or three of an RNF135 gene, a CHFR gene and a PAX5 gene.

Furthermore, the target sequences of the RNF135 gene, CHFR gene and PAX5 gene, and the primers and probes used are as described above.

In a specific embodiment, the data acquisition step comprises the following sub-steps: an extraction step, for extracting genomic DNA from a sample;
a reagent treatment step, for treating the genomic DNA extracted with a bisulfite reagent to convert the 5-unmethylated cytosine base to uracil or other bases;
a detection step, for detecting the genomic DNA treated with a bisulfite reagent to obtain the methylation marker level of the sample.

In a specific embodiment, in the detection step, the genomic DNA treated with a bisulfite reagent is detected using real-time fluorescent quantitative PCR.

In a specific embodiment, in the step of calculating the probability of suffering from liver cancer, a pre-constructed formula for calculating the probability of a subject suffering from liver cancer based on data fitting of methylation marker levels is pre-stored.

In a specific embodiment, the formula is obtained by fitting data on the methylation level of RNF135 in an existing database.

In a specific embodiment, the formula is obtained by fitting the data on the methylation level of RNF135 and the methylation level of PAX5 in an existing database.

In a specific embodiment, the formula is obtained by fitting the data on the methylation level of RNF135, the methylation level of PAX5, and the methylation level of CHFR in an existing database.

In a specific embodiment, the formula is obtained by fitting the data on the methylation level of RNF135, the methylation level of PAX5, and the methylation level of CHFR in the existing database using generalized linear regression. The generalized linear model is an extension of the linear model, which establishes the relationship between the mathematical expectation of the response variable and the linear combination of predictor variables through the link function. The main feature is that it does not forcibly change the natural measurement of the data. It is a commonly used binary classification strategy.

In a specific embodiment, the formula is:

$$P = {e}^{( a * M 1 + b * M 2 + c * M 3 + 81 )} / ( {e}^{( a * M 1 + b * M 2 + c * M 3 + 81 )} + 1 ) ,$$

wherein P represents the probability of the subject suffering from liver cancer, M1 represents the Ct value of RNF135 determined by real-time fluorescence quantitative PCR, M2 represents the Ct value of PAX5 determined by real-time fluorescence quantitative PCR, and M3 represents the Ct value of CHFR determined by real-time fluorescence quantitative PCR,
a is selected from any value between 0.8 and 1.2, preferably 0.9;
b is selected from any value between 0.6 and 0.9, preferably 0.7;
c is selected from any value between 0.1 and 0.5, preferably 0.3.

In a specific embodiment, when the probability that the subject suffers from liver cancer, that is, P, is greater than or equal to 0.24, the subject is determined to have liver cancer.

The inventors of the present application have found that there are significant differences between the methylation status of the target sequences of the CHFR gene, SPINT2 gene, RNF135 gene, CHST2 gene, SPDYA gene, PAX5 gene, VASH2 gene, TSPYL5 gene and EMX1 gene in liver cancer tissue and the methylation status of the target sequences of the above genes in normal liver tissue: in liver cancer tissue, the target sequences of the CHFR gene, SPINT2 gene, RNF135 gene, CHST2 gene, SPDYA gene, PAX5 gene, VASH2 gene, TSPYL5 gene and EMX1 gene will be methylated; while in normal liver tissue, the target sequences of the CHFR gene, SPINT2 gene, RNF135 gene, CHST2 gene, SPDYA gene, PAX5 gene, VASH2 gene, TSPYL5 gene and EMX1 gene will not be methylated. Therefore, the present application provides a method for detecting liver cancer in vitro by detecting the methylation status of target sequences of one or more genes of the CHFR gene, SPINT2 gene, RNF135 gene, CHST2 gene, SPDYA gene, PAX5 gene, VASH2 gene, TSPYL5 gene and EMX1 gene in a sample. The method provided in the present application can detect liver cancer non-invasively and rapidly.

### EXAMPLES

The present application provides a general and/or specific description of the materials and experimental methods used in the experiments. In the following Examples, unless otherwise specified, % represents wt %, i.e., weight percentage. The reagents or instruments used without indicating the manufacturer are all conventional reagent products that can be obtained through commercial purchase.

### Example 1 Primer and Probe Test

Firstly, sites with low methylation levels were screened in the next-generation sequencing information of 340 healthy human WBC samples and 100 healthy human plasma cfDNA samples. Secondly, the low methylation level sites screened out were analyzed for plasma and liver cancer tissues, and the sites that showed differences between the plasma of 377 liver cancer patients and the plasma of 200 normal people, and showed differences between 377 liver cancer tissues and 53 adjacent cancer tissues were screened out as candidate markers. Then, the candidate markers are traced back to the source of plasma. Finally, through sensitivity and specificity verification of liver cancer tissues and normal human plasma, nine specificity markers were identified in total, including CHFR gene, SPINT2 gene, RNF135 gene, CHST2 gene, SPDYA gene, PAX5 gene, VASH2 gene, TSPYL5 gene and EMX1 gene.

Primers and probes were designed based on the target sequences of the above nine genes. The designed primer and probe sequences are shown in Table 2 above.

Normal human leukocyte cell line DNA is usually in a low methylated/unmethylated state and can be used as a negative control. The amount of DNA used in this embodiment was 15.75 ng/reaction; fully methylated DNA is in a high/fully methylated state and can be used as a positive control. The amount of DNA used in this embodiment was 200 μg/reaction. The DNA sample was first subjected to bisulfite conversion, and the converted BisDNA was used as a template to perform real-time PCR amplification using the above primer probes. The β-actin (ACTB) gene was used as an internal reference, and a β-actin gene amplicon was created by using primers complementary to the β-actin gene sequence, and the β-actin gene amplicon was detected with a specific probe. Real-time PCR was performed at least once for each sample, and in some specific embodiments, twice or three times. The PCR system for the primer-probe test is shown in Table 4 below.

TABLE 4
PCR system for primer and probe test.

| | Volume (μL) | Final concentration |
|---|---|---|
| Taq DNA Polymerase (Biochain) | 1.2 | / |
| 4.2 × buffer (Biochain) | 11.9 | 1× |
| forward primer F (10 μM) | 1.0 | 200 nM |
| reverse primer R (10 μM) | 1.0 | 200 nM |
| Probe P (10 μM) | 1.0 | 200 nM |
| ACTB_F (10 μM) | 0.5 | 100 nM |
| ACTB_R (10 μM) | 0.5 | 100 nM |
| ACTB_P (10 μM) | 0.375 | 75 nM |
| BisDNA | 4.0 | / |
| H₂O | 28.525 | / |
| Total | 50.0 | / |

Note:
“F” indicates forward primer; “R” indicates reverse primer; “P” indicates probe.

The PCR amplification program used was: 94° C., 20 min; 45 cycles (93° C., 30 s; 57° C., 35 s-read the fluorescence signal); 40° C., 5 s.

The results are shown in Table 5. When BisDNA of fully methylated DNA was used as a template, the CHFR gene, SPINT2 gene, RNF135 gene, CHST2 gene, SPDYA gene, PAX5 gene, VASH2 gene, TSPYL5 gene and EMX1 gene were all effectively amplified; however, when BisDNA of WBC was used as a template, except the internal reference ACTB gene, all the other target genes were not amplified.

TABLE 5
Test results of primers and probes for each gene.

| Sample type | Test primer and probe | Ct value |
|---|---|---|
| Positive control | CHFR_1 | 34.09 |
| | ACTB | 33.67 |
| Negative control | CHFR_1 | NoCt |
| | ACTB | 26.95 |
| Positive control | SPINT2_1 | 33.36 |
| | ACTB | 33.45 |
| Negative control | SPINT2_1 | NoCt |
| | ACTB | 26.47 |
| Positive control | RNF135_1 | 33.21 |
| | ACTB | 33.67 |
| Negative control | RNF135_1 | NoCt |
| | ACTB | 26.57 |
| Positive control | CHST2_1 | 34.70 |
| | ACTB | 35.71 |
| Negative control | CHST2_1 | NoCt |
| | ACTB | 26.33 |
| Positive control | SPDYA_1 | 33.31 |
| | ACTB | 32.62 |
| Negative control | SPDYA_1 | NoCt |
| | ACTB | 26.27 |
| Positive control | PAX5_1 | 33.67 |
| | ACTB | 36.11 |
| Negative control | PAX5_1 | NoCt |
| | ACTB | 26.94 |
| Positive control | VASH2_1 | 32.89 |
| | ACTB | 33.97 |
| Negative control | VASH2_1 | NoCt |
| | ACTB | 26.93 |
| Positive control | TSPYL5_1 | 33.64 |
| | ACTB | 35.19 |
| Negative control | TSPYL5_1 | NoCt |
| | ACTB | 26.62 |
| Positive control | EMX1_1 | 33.44 |
| | ACTB | 35.15 |
| Negative control | EMX1_1 | NoCt |
| | ACTB | 26.80 |

wherein “No Ct” means no Ct value was detected.

### Example 2

16 Liver cancer tissue samples (10 ng/reaction) and 16 normal human plasma samples (3.5 mL) were selected, and genomic DNA was extracted. After converting to BisDNA via bisulfite, the methylation of the CHFR gene, SPINT2 gene, RNF135 gene, CHST2 gene, SPDYA gene, PAX5 gene, VASH2 gene, TSPYL5 gene, and EMX1 gene was detected according to the PCR reaction system and reaction procedure in Example 1. The threshold of each marker was set at 41, and the Ct values of real-time PCR for the target sequence of the target gene were finally measured for 16 liver cancer tissues and 16 normal human plasma.

The test results are shown in Table 6, wherein two or more of the six genes, including RNF135 gene, CHFR gene, SPINT2 gene, SPDYA gene, PAX5 gene and VASH2 gene, were detected jointly and any one of the genes which were tested to be positive was interpreted as positive.

TABLE 6
Sensitivity/specificity of each gene in liver
cancer tissue and normal human plasma.

| | Liver | Normal |
|---|---|---|
| | cancer | human |
| | tissue | plasma |
| Total number of samples | 16 | 16 |
| Number of CHFR positive detections | 15 | 1 |
| Number of SPINT2 positive detections | 12 | 1 |
| Number of RNF135 positive detections | 14 | 0 |
| Number of CHST2 positive detections | 14 | 2 |
| Number of SPDYA positive detections | 14 | 0 |
| Number of PAX5 positive detections | 9 | 1 |
| Number of VASH2 positive detections | 14 | 0 |
| Number of TSPYL5 positive detections | 12 | 3 |
| Number of EMX1 positive detections | 11 | 3 |
| Number of RNF135 + CHFR positive detections | 16 | 1 |
| Number of RNF135 + PAX5 positive detections | 15 | 1 |
| Number of CHFR + PAX5 positive detections | 15 | 2 |
| Number of RNF135 + CHFR + PAX5 positive | 16 | 2 |
| detections | | |
| Number of RNF135 + CHFR + PAX5 + | 16 | 3 |
| SPINT2 + SPDYA + VASH2 positive detections | | |
| CHFR detection sensitivity/specificity | 93.75% | 93.75% |
| SPINT2 detection sensitivity/specificity | 75% | 93.75% |
| RNF135 detection sensitivity/specificity | 87.50% | 100% |
| CHST2 detection sensitivity/specificity | 87.50% | 87.50% |
| SPDYA detection sensitivity/specificity | 87.50% | 100% |
| PAX5 detection sensitivity/specificity | 56.25% | 93.75% |
| VASH2 detection sensitivity/specificity | 87.50% | 100% |
| TSPYL5 detection sensitivity/specificity | 75% | 81.25% |
| EMX1 detection sensitivity/specificity | 68.75% | 81.25% |
| RNF135 + CHFR detection sensitivity/specificity | 100% | 100% |
| RNF135 + PAX5 detection sensitivity/specificity | 93.75% | 93.75% |
| CHFR + PAX5 detection sensitivity/specificity | 93.75% | 93.75% |
| RNF135 + CHFR + PAX5 detection sensitivity/ | 100% | 100% |
| specificity | | |
| RNF135 + CHFR + PAX5 + SPINT2 + | 100% | 81.25% |
| SPDYA + VASH2 detection sensitivity/specificity | | |

The results in Table 6 showed that the sensitivity of detecting liver cancer tissue using CHFR gene, SPINT2 gene, RNF135 gene, CHST2 gene, SPDYA gene, PAX5 gene, VASH2 gene, TSPYL5 gene and EMX1 gene were 93.75%, 75%, 87.5%, 87.5%, 87.5%, 56.25%, 87.5%, 75% and 68.75% respectively; the sensitivity of joint detection of six genes including CHFR gene, SPINT2 gene, RNF135 gene, CHST2 gene, SPDYA gene, PAX5 gene, VASH2 gene, TSPYL5 gene and EMX1 gene reached 100%; the sensitivity of joint detection of three genes including RNF135 gene, CHFR gene and PAX5 gene reached 100%.

The methylation of the target sequence of the target gene had good specificity. The specificities of CHFR gene, SPINT2 gene, RNF135 gene, CHST2 gene, SPDYA gene, PAX5 gene, VASH2 gene, TSPYL5 gene and EMX1 gene in normal human plasma were 93.75%, 93.75%, 100%, 87.5%, 100%, 93.75%, 100%, 81.25% and 81.25%, respectively; the specificity of joint detection of six genes, including CHFR gene, SPINT2 gene, RNF135 gene, SPDYA gene, PAX5 gene and VASH2 gene, was 81.25%; the specificity of joint detection of three genes, including RNF135 gene, CHFR gene and PAX5 gene, was 87.5%.

Among the above-mentioned 9 markers related to liver cancer, it can be seen from the tissue detection results that, 5 markers had a tissue sensitivity of more than 80%, i.e., CHFR gene, RNF135 gene, CHST2 gene, SPDYA gene and VASH2 gene. The CHFR gene had the best tissue sensitivity, reaching 93.75%. It can be seen from the test results of normal human plasma that, 6 markers had a plasma specificity of above 90%, i.e., CHFR gene, SPINT2 gene, RNF135 gene, SPDYA gene, PAX5 gene and VASH2 gene. Under the conditions of small sample size verification, a plasma specificity reaching 80% represents a relatively reasonable and high indicator level.

### Example 3

Ten liver cancer patient plasma samples (3.5 mL) and 16 normal human plasma samples (3.5 mL) were selected, and genomic DNA was extracted. After converting to BisDNA via bisulfite, the methylation of the RNF135, CHFR, SPINT2, SPDYA, PAX5, and VASH2 genes was detected using the PCR reaction system described in Example 2. The threshold for each gene was set at 41, and the Ct values of real-time PCR for the target sequence of the target gene were finally measured for 10 liver cancer patient plasma samples and 16 normal human plasma samples. The PCR results are shown in Table 7.

TABLE 7
Test results of each gene in cancer patient
plasma and normal human plasma.

| | Liver cancer | Normal human | Liver | |
|---|---|---|---|---|
| | patient plasma | plasma | cancer | Normal |
| | Number of | Number of | patient | human |
| | positive | positive | plasma | plasma |
| | detected | detected | Sensi- | Speci- |
| | cases | cases | tivity | ficity |
| Total number of | 10 | 16 | / | / |
| samples | | | | |
| RNF135 | 8 | 0 | 80% | 100% |
| CHFR | 7 | 1 | 70% | 93.75% |
| SPINT2 | 6 | 0 | 60% | 100% |
| SPDYA | 7 | 1 | 70% | 93.75% |
| PAX5 | 4 | 1 | 40% | 93.75% |
| VASH2 | 4 | 0 | 40% | 100% |
| the joint detection | 9 | 3 | 90% | 81.25% |
| of 6 genes, | | | | |
| RNF135 + CHFR + | | | | |
| PAX5 + SPINT2 + | | | | |
| SPDYA + VASH2 | | | | |

The results in Table 7 showed that the sensitivities of RNF135 gene, CHFR gene, SPINT2 gene, SPDYA gene, PAX5 gene and VASH2 gene alone for detecting liver cancer patient plasma were 80%, 70%, 60%, 70%, 40% and 40%, respectively. The specificities of the six genes alone for detecting normal human plasma are 100%, 93.75%, 100%, 93.75%, 93.75% and 100%, respectively.

It can be seen from the above-mentioned liver cancer patient plasma sensitivity and normal human plasma specificity test results that, in the case that the specificity remained above 90%, three markers showed a sensitivity of above 70%, but only the RNF135 gene showed a sensitivity of reaching 80%. The sensitivity of the joint detection of 6 genes can reach 90%. Therefore, the joint detection of 6 genes can improve the sensitivity to a certain extent.

### Example 4

26 Liver cancer patient plasma samples (3.5 mL) and 38 normal human plasma samples (3.5 mL) were selected, genomic DNA was extracted. After converting to BisDNA via bisulfite, the methylation of the RNF135, CHFR, and PAX5 genes was detected using the PCR reaction system described in Example 2. The threshold of the three genes was set at 41, and the Ct values of the real-time PCR for the target sequences of the target genes were finally measured for 26 liver cancer patient plasma samples and 38 normal human plasma samples.

The test results are shown in Table 8. The sensitivity of RNF135 gene, CHFR gene and PAX5 gene alone for detecting liver cancer patient plasma samples were 80.77%, 65.39% and 30.77% respectively. Among them, the sensitivity of RNF135 gene for detecting liver cancer patient plasma samples was the best, reaching 80.77%. Although the sensitivity of CHFR gene and PAX5 gene alone for detecting liver cancer plasma samples was only 30%-65%, the sensitivity of RNF135 gene, CHFR gene and PAX5 gene for joint detecting liver cancer patient plasma samples can reach 92.31%. As shown in Table 9, the specificities of RNF135 gene, CHFR gene and PAX5 gene alone for detecting normal human plasma were 94.74%, 92.11% and 94.74%, respectively. Among them, RNF135 gene and PAX5 gene had the highest specificities, reaching 94.74%. The specificity of the joint detection of the three genes reached 84.21%.

In summary, the specificity of joint detection of RNF135, CHFR and PAX5 was lower than that of single gene detection, but its specificity also reached 84%. Particularly importantly, the sensitivity of RNF135, CHFR and PAX5 genes for joint detecting liver cancer patient plasma samples can reach 92.31%, which was significantly improved compared to the sensitivity of single gene for testing liver cancer patient plasma samples. Therefore, when RNF135 gene, CHFR gene and PAX5 gene were used for detecting liver cancer in vitro in combination, they can effectively improve the sensitivity of in vitro detection of liver cancer.

TABLE 8
Sensitivity/specificity of RNF135, CHFR and PAX5 genes
in liver cancer patient plasma and normal human plasma.

| | Liver cancer | Normal |
|---|---|---|
| | patient | human |
| | plasma | plasma |
| Number of samples | 26 | 38 |
| Number of RNF135 positive detections | 21 | 2 |
| Number of CHFR positive detections | 17 | 3 |
| Number of PAX5 positive detections | 8 | 2 |
| Number of RNF135 + CHFR positive detections | 23 | 5 |
| Number of RNF135 + PAX5 positive detections | 23 | 4 |
| Number of CHFR + PAX5 positive detections | 19 | 5 |
| Number of RNF135 + CHFR + PAX5 positive | 24 | 6 |
| detections | | |
| RNF135 detection sensitivity/specificity | 80.77% | 94.74% |
| CHFR detection sensitivity/specificity | 65.39% | 92.11% |
| PAX5 detection sensitivity/specificity | 30.77% | 94.74% |
| RNF135 + CHFR detection sensitivity/specificity | 88.46% | 86.84% |
| RNF135 + PAX5 detection sensitivity/specificity | 88.46% | 89.47% |
| CHFR + PAX5 detection sensitivity/specificity | 73.08% | 86.84% |
| RNF135 + CHFR + PAX5 detection | 92.31% | 84.21% |
| sensitivity/specificity | | |

### Example 5

The Ct value data of 64 samples (26 liver cancer patient plasma samples and 38 normal human plasma samples) in Example 4 were used as the training data set for the model fitting. All the samples were split into training test and test set at a proportion of 75:25. A 5-fold cross-validation was performed on the training set. Different marker types (including differentially methylated regions, methylation haplotypes, etc.) were combined with different marker screening strategies (including screening indicators such as AUC and mutual information) and modeling methods (such as random forest, support vector machine, generalized linear model, XGboost, etc.) to perform marker screening and modeling. The above method combinations were evaluated based on AUC as a standard. Finally, the formula was fitted in a generalized linear regression manner, resulting in the following formula:

that is, P=e^{(0.9*M1+0.7*M2+0.3*M3+81)}/(e^{(0.9*M1+0.7*M2+0.3*M3+81)}+1),

Among them, P represents the probability of the subject suffering from liver cancer, M1 represents the Ct value of RNF135 in real-time fluorescence quantitative PCR detection, M2 represents the Ct value of PAX5 in real-time fluorescence quantitative PCR detection, and M3 represents the Ct value of CHFR in real-time fluorescence quantitative PCR detection.

The Ct values of the RNF135, PAX5, and CHFR test samples were respectively substituted into the above interpretation formula. The specified threshold (Cut-off value) of the interpretation formula was 0.24, that is, when P<0.24, the patient was negative, and when P≥0.24, the patient was positive.

### Example 6

A total of 116 samples (62 liver cancer patient plasma samples and 54 normal human plasma samples) were collected, and methylation detection of the RNF135 gene, CHFR gene, and PAX5 gene was performed according to the PCR reaction system in Example 2. The threshold of the Ct value of the three genes was set at 41, and finally the Ct values for the target sequence of the target gene were measured by real-time PCR in 116 samples. The Ct value data of RNF135, PAX5 and CHFR were calculated for P value by using the interpretation formula of Example 5, and the P value was compared with the specified threshold of the formula. The results of the solutions which only used joint detection of genes (Method 1, i.e., as for the joint interpretation of the three genes RNF135, CHFR and PAX5, if any one of the genes was detected to be positive, it was determined as positive), and the solutions which used joint detection of genes+formula interpretation (Method 2, i.e., joint detection of the three genes RNF135, CHFR and PAX5, and interpretation according to the formula in Example 5) are shown in Table 9.

TABLE 9
Detection sensitivity and specificity of Method 1 and Method
2 in liver cancer patient plasma and normal human plasma.

| | Liver cancer | Normal |
|---|---|---|
| | patient | human |
| | plasma | plasma |
| Number of samples | 62 | 54 |
| Method 1: Number of positive detections | 58 | 9 |
| Method 2: Number of positive detections | 58 | 5 |
| Method 1: Detection sensitivity/specificity | 93.55% | 83.33% |
| Method 2: Detection sensitivity/specificity | 93.55% | 90.74% |

It can be seen from the above test results that the sensitivity of RNF135, CHFR and PAX5 for joint detecting liver cancer patient plasma was 93.55%, and the specificity of RNF135, CHFR and PAX5 for joint detecting normal human plasma was 83.33%. After introducing the interpretation formula, the sensitivity of detecting liver cancer patient plasma was 93.55%, and the specificity of detecting normal human plasma was 90.74%. Compared to using only joint detection, adding an interpretation formula to interpret the test results can improve its specificity without reducing the sensitivity.

The above tests showed that the interpretation formula can be used for early diagnosis of liver cancer, and its reference value will improve specificity to a certain extent.

The above description is only preferred Examples of the present application and is not intended to impose any other form of limitation on the present application. Any skilled technician familiar with the art may use the technical content disclosed above to change or modify it into an equivalent embodiment with equivalent changes. However, any simple modifications, equivalent changes and modifications made to the above examples based on the technical essence of the present application without departing from the content of the technical solution of the present application still fall within the scope of protection of the technical solution of the present application.

1-78. (canceled)

79. A composition for detecting liver cancer in vitro, comprising:
a nucleic acid used for detecting the methylation status of a target gene,
wherein, the methylation status of the target gene is characterized by the methylation of the target sequence of the target gene,
wherein, the target gene is one or more of: a CHFR gene, an SPINT2 gene, an RNF135 gene, a CHST2 gene, an SPDYA gene, a PAX5 gene, a VASH2 gene, a TSPYL5 gene, and an EMX1 gene.

80. The composition according to claim 79, wherein the target sequence of the CHFR gene is shown as SEQ ID NO: 1 or SEQ ID NO: 2 or SEQ ID NO: 3 or SEQ ID NO: 4, or comprises the sequence shown as SEQ ID NO: 1 or SEQ ID NO: 2 or SEQ ID NO: 3 or SEQ ID NO: 4; or
wherein the target sequence of the SPINT2 gene is shown as SEQ ID NO: 5 or SEQ ID NO: 6 or SEQ ID NO: 7 or SEQ ID NO: 8, or comprises the sequence shown as SEQ ID NO: 5 or SEQ ID NO: 6 or SEQ ID NO: 7 or SEQ ID NO: 8; or
wherein the target sequence of the RNF135 gene is shown as SEQ ID NO: 9 or SEQ ID NO: 10 or SEQ ID NO: 11 or SEQ ID NO: 12, or comprises the sequence shown as SEQ ID NO: 9 or SEQ ID NO: 10 or SEQ ID NO: 11 or SEQ ID NO: 12; or
wherein the target sequence of the CHST2 gene is shown as SEQ ID NO: 13 or SEQ ID NO: 14 or SEQ ID NO: 15 or SEQ ID NO: 16, or comprises the sequence shown as SEQ ID NO: 13 or SEQ ID NO: 14 or SEQ ID NO: 15 or SEQ ID NO: 16; or
wherein the target sequence of the SPDYA gene is shown as SEQ ID NO: 17 or SEQ ID NO: 18 or SEQ ID NO: 19 or SEQ ID NO: 20, or comprises the sequence shown as SEQ ID NO: 17 or SEQ ID NO: 18 or SEQ ID NO: 19 or SEQ ID NO: 20; or
wherein the target sequence of the PAX5 gene is shown as SEQ ID NO: 21 or SEQ ID NO: 22 or SEQ ID NO: 23 or SEQ ID NO: 24, or comprises the sequence shown as SEQ ID NO: 21 or SEQ ID NO: 22 or SEQ ID NO: 23 or SEQ ID NO: 24; or
wherein the target sequence of the VASH2 gene is shown as SEQ ID NO: 25 or SEQ ID NO: 26 or SEQ ID NO: 27 or SEQ ID NO: 28, or comprises the sequence shown as SEQ ID NO: 25 or SEQ ID NO: 26 or SEQ ID NO: 27 or SEQ ID NO: 28; or
wherein the target sequence of the TSPYL5 gene is shown as SEQ ID NO: 29 or SEQ ID NO: 30 or SEQ ID NO: 31 or SEQ ID NO: 32, or comprises the sequence shown as SEQ ID NO: 29 or SEQ ID NO: 30 or SEQ ID NO: 31 or SEQ ID NO: 32; or
wherein the target sequence of the EMX1 gene is shown as SEQ ID NO: 33 or SEQ ID NO: 34 or SEQ ID NO: 35 or SEQ ID NO: 36, or comprises the sequence shown as SEQ ID NO: 33 or SEQ ID NO: 34 or SEQ ID NO: 35 or SEQ ID NO: 36.

81. The composition according to claim 79, wherein the nucleic acid for detecting the methylation status of the target gene comprises:
a primer, wherein the primer is a fragment of at least 9 nucleotides in the target sequence of the target gene,
the fragment comprises at least one CpG dinucleotide sequence;
preferably, wherein the nucleic acid for detecting the methylation status of the target gene further comprises:
a probe, wherein the probe is a fragment that hybridizes to at least 15 nucleotides of the target sequence of the target gene under moderately stringent or stringent conditions, the fragment comprises at least one CpG dinucleotide sequence.

82. The composition according to claim 79, further comprising:
a reagent that converts 5-unmethylated cytosine base of the target sequence of the target gene to uracil,
preferably, wherein the nucleic acid for detecting the methylation status of the target gene further comprises:
a blocker that preferentially binds to the target sequence in its unmethylated state.

83. The composition according to claim 81, wherein
the fragment of at least 9 nucleotides is a sequence of SEQ ID NO:37 and SEQ ID NO:38, or a sequence of SEQ ID NO:40 and SEQ ID NO:41, or a sequence of SEQ ID NO:43 and SEQ ID NO: 44, or a sequence of SEQ ID NO:46 and SEQ ID NO:47, or a sequence of SEQ ID NO: 49 and SEQ ID NO:50, or a sequence of SEQ ID NO:52 and SEQ ID NO:53, a sequence of SEQ ID NO:55 and SEQ ID NO:56, or a sequence of SEQ ID NO:58 and SEQ ID NO:59, or a sequence of SEQ ID NO:61 and SEQ ID NO:62;
the fragment of at least 15 nucleotides is a sequence of SEQ ID NO:39, or a sequence of SEQ ID NO:42, or a sequence of SEQ ID NO:45, or a sequence of SEQ ID NO:48, or a sequence of SEQ ID NO:51, or a sequence of SEQ ID NO:54, or a sequence of SEQ ID NO:57, or a sequence of SEQ ID NO:60, or a sequence of SEQ ID NO:63.

84. An oligonucleotide for detecting liver cancer in vitro, comprising:
a fragment of at least 9 nucleotides of SEQ ID NO: 1 or SEQ ID NO: 2 or SEQ ID NO: 3 or SEQ ID NO: 4 or a complementary sequence thereof and comprising at least one CpG dinucleotide sequence; and/or
a fragment of at least 9 nucleotides of SEQ ID NO: 5 or SEQ ID NO: 6 or SEQ ID NO: 7 or SEQ ID NO: 8 or a complementary sequence thereof and comprising at least one CpG dinucleotide sequence; and/or
a fragment of at least 9 nucleotides of SEQ ID NO: 9 or SEQ ID NO: 10 or SEQ ID NO: 11 or SEQ ID NO: 12 or a complementary sequence thereof and comprising at least one CpG dinucleotide sequence; and/or
a fragment of at least 9 nucleotides of SEQ ID NO: 13 or SEQ ID NO: 14 or SEQ ID NO: 15 or SEQ ID NO: 16 or a complementary sequence thereof and comprising at least one CpG dinucleotide sequence; and/or
a fragment of at least 9 nucleotides of SEQ ID NO: 17 or SEQ ID NO: 18 or SEQ ID NO: 19 or SEQ ID NO: 20 or a complementary sequence thereof and comprising at least one CpG dinucleotide sequence; and/or
a fragment of at least 9 nucleotides of SEQ ID NO: 21 or SEQ ID NO: 22 or SEQ ID NO: 23 or SEQ ID NO: 24 or a complementary sequence thereof and comprising at least one CpG dinucleotide sequence; and/or
a fragment of at least 9 nucleotides of SEQ ID NO: 25 or SEQ ID NO: 26 or SEQ ID NO: 27 or SEQ ID NO: 28 or a complementary sequence thereof and comprising at least one CpG dinucleotide sequence; and/or
a fragment of at least 9 nucleotides of SEQ ID NO: 29 or SEQ ID NO: 30 or SEQ ID NO: 31 or SEQ ID NO: 32 or a complementary sequence thereof and comprising at least one CpG dinucleotide sequence; and/or
a fragment of at least 9 nucleotides of SEQ ID NO: 33 or SEQ ID NO: 34 or SEQ ID NO: 35 or SEQ ID NO: 36 or a complementary sequence thereof and comprising at least one CpG dinucleotide sequence;
preferably further comprising:
a fragment that hybridizes to at least 15 nucleotides of SEQ ID NO: 1 or SEQ ID NO: 2 or SEQ ID NO: 3 or SEQ ID NO: 4 or a complementary sequence thereof under moderately stringent or stringent conditions and comprises at least one CpG dinucleotide sequence; and/or
a fragment that hybridizes to at least 15 nucleotides of SEQ ID NO: 5 or SEQ ID NO: 6 or SEQ ID NO: 7 or SEQ ID NO: 8 or a complementary sequence thereof under moderately stringent or stringent conditions and comprises at least one CpG dinucleotide sequence; and/or
a fragment that hybridizes to at least 15 nucleotides of SEQ ID NO: 9 or SEQ ID NO: 10 or SEQ ID NO: 11 or SEQ ID NO: 12 or a complementary sequence thereof under moderately stringent or stringent conditions and comprises at least one CpG dinucleotide sequence; and/or
a fragment that hybridizes to at least 15 nucleotides of SEQ ID NO: 13 or SEQ ID NO: 14 or SEQ ID NO: 15 or SEQ ID NO: 16 or a complementary sequence thereof under moderately stringent or stringent conditions and comprises at least one CpG dinucleotide sequence; and/or
a fragment that hybridizes to at least 15 nucleotides of SEQ ID NO: 17 or SEQ ID NO: 18 or SEQ ID NO: 19 or SEQ ID NO: 20 or a complementary sequence thereof under moderately stringent or stringent conditions and comprises at least one CpG dinucleotide sequence; and/or
a fragment that hybridizes to at least 15 nucleotides of SEQ ID NO: 21 or SEQ ID NO: 22 or SEQ ID NO: 23 or SEQ ID NO: 24 or a complementary sequence thereof under moderately stringent or stringent conditions and comprises at least one CpG dinucleotide sequence; and/or
a fragment that hybridizes to at least 15 nucleotides of SEQ ID NO: 25 or SEQ ID NO: 26 or SEQ ID NO: 27 or SEQ ID NO: 28 or a complementary sequence thereof under moderately stringent or stringent conditions and comprises at least one CpG dinucleotide sequence; and/or
a fragment that hybridizes to at least 15 nucleotides of SEQ ID NO: 29 or SEQ ID NO: 30 or SEQ ID NO: 31 or SEQ ID NO: 32 or a complementary sequence thereof under moderately stringent or stringent conditions and comprises at least one CpG dinucleotide sequence; and/or
a fragment that hybridizes to at least 15 nucleotides of SEQ ID NO: 33, SEQ ID NO: 34, SEQ ID NO: 35, or SEQ ID NO: 36 or a complementary sequence thereof under moderately stringent or stringent conditions and comprises at least one CpG dinucleotide sequence;
more preferably further comprising:
a blocker that preferentially binds to the target sequence in its unmethylated state.

85. The oligonucleotide according to claim 84, comprising:
the sequences of SEQ ID NO: 37 and SEQ ID NO: 38; or the sequences of SEQ ID NO: 40 and SEQ ID NO: 41; or the sequences of SEQ ID NO: 43 and SEQ ID NO: 44; or the sequences of SEQ ID NO: 46 and SEQ ID NO: 47; or the sequences of SEQ ID NO: 49 and SEQ ID NO: 50; or the sequences of SEQ ID NO: 52 and SEQ ID NO: 53; or the sequences of SEQ ID NO: 55 and SEQ ID NO: 56; or the sequences of SEQ ID NO: 58 and SEQ ID NO: 59; or the sequences of SEQ ID NO: 61 and SEQ ID NO: 62;
preferably further comprising:
the sequence of SEQ ID NO: 39; or the sequence of SEQ ID NO: 42; or the sequence of SEQ ID NO: 45; or the sequence of SEQ ID NO: 48; or the sequence of SEQ ID NO: 51; or the sequence of SEQ ID NO: 54; or the sequence of SEQ ID NO: 57; or the sequence of SEQ ID NO: 60; or the sequence of SEQ ID NO: 63.

86. A method for detecting liver cancer, comprising the following steps:
isolating a DNA sample comprising a target sequence of a target gene or a fragment thereof from a biological sample to be tested;
determining the methylation status of the target sequence of the target gene; and
determining the state of the biological sample based on the detection result of the methylation status of the target sequence of the target gene, thereby realizing in vitro detection of liver cancer; or
comprising the following steps:
extracting genomic DNA from the biological sample to be tested;
treating the extracted genomic DNA with a reagent to convert the 5-unmethylated cytosine base to uracil or other bases;
contacting the DNA sample treated with the reagent with a DNA polymerase and a primer of a target sequence of a target gene to initiate a DNA polymerization reaction;
detecting the amplified product with a probe; and
determining the methylation status of at least one CpG dinucleotide of the target sequence of the target gene based on whether the amplified product is present or not,
preferably wherein the target gene is one or more of: a CHFR gene, an SPINT2 gene, an RNF135 gene, a CHST2 gene, an SPDYA gene, a PAX5 gene, a VASH2 gene, a TSPYL5 gene and an EMX1 gene.

87. The method according to claim 86, wherein the target sequence of the CHFR gene is shown as SEQ ID NO: 1 or SEQ ID NO: 2 or SEQ ID NO: 3 or SEQ ID NO: 4, or comprises the sequence shown as SEQ ID NO: 1 or SEQ ID NO: 2 or SEQ ID NO: 3 or SEQ ID NO: 4; or
wherein the target sequence of the SPINT2 gene is shown as SEQ ID NO: 5 or SEQ ID NO: 6 or SEQ ID NO: 7 or SEQ ID NO: 8, or comprises the sequence shown as SEQ ID NO: 5 or SEQ ID NO: 6 or SEQ ID NO: 7 or SEQ ID NO: 8; or
wherein the target sequence of the RNF135 gene is shown as SEQ ID NO: 9 or SEQ ID NO: 10 or SEQ ID NO: 11 or SEQ ID NO: 12, or comprises the sequence shown as SEQ ID NO: 9 or SEQ ID NO: 10 or SEQ ID NO: 11 or SEQ ID NO: 12; or
wherein the target sequence of the CHST2 gene is shown as SEQ ID NO: 13 or SEQ ID NO: 14 or SEQ ID NO: 15 or SEQ ID NO: 16, or comprises the sequence shown as SEQ ID NO: 13 or SEQ ID NO: 14 or SEQ ID NO: 15 or SEQ ID NO: 16; or
wherein the target sequence of the SPDYA gene is shown as SEQ ID NO: 17 or SEQ ID NO: 18 or SEQ ID NO: 19 or SEQ ID NO: 20, or comprises the sequence shown as SEQ ID NO: 17 or SEQ ID NO: 18 or SEQ ID NO: 19 or SEQ ID NO: 20; or
wherein the target sequence of the PAX5 gene is shown as SEQ ID NO: 21 or SEQ ID NO: 22 or SEQ ID NO: 23 or SEQ ID NO: 24, or comprises the sequence shown as SEQ ID NO: 21 or SEQ ID NO: 22 or SEQ ID NO: 23 or SEQ ID NO: 24; or
wherein the target sequence of the VASH2 gene is shown as SEQ ID NO: 25 or SEQ ID NO: 26 or SEQ ID NO: 27 or SEQ ID NO: 28, or comprises the sequence shown as SEQ ID NO: 25 or SEQ ID NO: 26 or SEQ ID NO: 27 or SEQ ID NO: 28; or
wherein the target sequence of the TSPYL5 gene is shown as SEQ ID NO: 29 or SEQ ID NO: 30 or SEQ ID NO: 31 or SEQ ID NO: 32, or comprises the sequence shown as SEQ ID NO: 29 or SEQ ID NO: 30 or SEQ ID NO: 31 or SEQ ID NO: 32; or
wherein the target sequence of the EMX1 gene is shown as SEQ ID NO: 33 or SEQ ID NO: 34 or SEQ ID NO: 35 or SEQ ID NO: 36, or comprises the sequence shown as SEQ ID NO: 33 or SEQ ID NO: 34 or SEQ ID NO: 35 or SEQ ID NO: 36.

88. The method according to claim 86, wherein the reagent is a bisulfite reagent.

89. The method according to claim 86, wherein the primer is:
a fragment of at least 9 nucleotides of SEQ ID NO: 1 or SEQ ID NO: 2 or SEQ ID NO: 3 or SEQ ID NO: 4 or a complementary sequence thereof and comprising at least one CpG dinucleotide sequence; and/or
a fragment of at least 9 nucleotides of SEQ ID NO: 5 or SEQ ID NO: 6 or SEQ ID NO: 7 or SEQ ID NO: 8 or a complementary sequence thereof and comprising at least one CpG dinucleotide sequence; and/or
a fragment of at least 9 nucleotides of SEQ ID NO: 9 or SEQ ID NO: 10 or SEQ ID NO: 11 or SEQ ID NO: 12 or a complementary sequence thereof and comprising at least one CpG dinucleotide sequence; and/or
a fragment of at least 9 nucleotides of SEQ ID NO: 13 or SEQ ID NO: 14 or SEQ ID NO: 15 or SEQ ID NO: 16 or a complementary sequence thereof and comprising at least one CpG dinucleotide sequence; and/or
a fragment of at least 9 nucleotides of SEQ ID NO: 17 or SEQ ID NO: 18 or SEQ ID NO: 19 or SEQ ID NO: 20 or a complementary sequence thereof and comprising at least one CpG dinucleotide sequence; and/or
a fragment of at least 9 nucleotides of SEQ ID NO: 21 or SEQ ID NO: 22 or SEQ ID NO: 23 or SEQ ID NO: 24 or a complementary sequence thereof and comprising at least one CpG dinucleotide sequence; and/or
a fragment of at least 9 nucleotides of SEQ ID NO: 25 or SEQ ID NO: 26 or SEQ ID NO: 27 or SEQ ID NO: 28 or a complementary sequence thereof and comprising at least one CpG dinucleotide sequence; and/or
a fragment of at least 9 nucleotides of SEQ ID NO: 29 or SEQ ID NO: 30 or SEQ ID NO: 31 or SEQ ID NO: 32 or a complementary sequence thereof and comprising at least one CpG dinucleotide sequence; and/or
a fragment of at least 9 nucleotides of SEQ ID NO: 33 or SEQ ID NO: 34 or SEQ ID NO: 35 or SEQ ID NO: 36 or a complementary sequence thereof and comprising at least one CpG dinucleotide sequence;
preferably, wherein the probe is:
a fragment that hybridizes to at least 15 nucleotides of SEQ ID NO: 1 or SEQ ID NO: 2 or SEQ ID NO: 3 or SEQ ID NO: 4 or a complementary sequence thereof under moderately stringent or stringent conditions and comprises at least one CpG dinucleotide sequence; and/or
a fragment that hybridizes to at least 15 nucleotides of SEQ ID NO: 5 or SEQ ID NO: 6 or SEQ ID NO: 7 or SEQ ID NO: 8 or a complementary sequence thereof under moderately stringent or stringent conditions and comprises at least one CpG dinucleotide sequence; and/or
a fragment that hybridizes to at least 15 nucleotides of SEQ ID NO: 9 or SEQ ID NO: 10 or SEQ ID NO: 11 or SEQ ID NO: 12 or a complementary sequence thereof under moderately stringent or stringent conditions and comprises at least one CpG dinucleotide sequence; and/or
a fragment that hybridizes to at least 15 nucleotides of SEQ ID NO: 13 or SEQ ID NO: 14 or SEQ ID NO: 15 or SEQ ID NO: 16 or a complementary sequence thereof under moderately stringent or stringent conditions and comprises at least one CpG dinucleotide sequence; and/or
a fragment that hybridizes to at least 15 nucleotides of SEQ ID NO: 17 or SEQ ID NO: 18 or SEQ ID NO: 19 or SEQ ID NO: 20 or a complementary sequence thereof under moderately stringent or stringent conditions and comprises at least one CpG dinucleotide sequence; and/or
a fragment that hybridizes to at least 15 nucleotides of SEQ ID NO: 21 or SEQ ID NO: 22 or SEQ ID NO: 23 or SEQ ID NO: 24 or a complementary sequence thereof under moderately stringent or stringent conditions and comprises at least one CpG dinucleotide sequence; and/or
a fragment that hybridizes to at least 15 nucleotides of SEQ ID NO: 25 or SEQ ID NO: 26 or SEQ ID NO: 27 or SEQ ID NO: 28 or a complementary sequence thereof under moderately stringent or stringent conditions and comprises at least one CpG dinucleotide sequence; and/or
a fragment that hybridizes to at least 15 nucleotides of SEQ ID NO: 29 or SEQ ID NO: 30 or SEQ ID NO: 31 or SEQ ID NO: 32 or a complementary sequence thereof under moderately stringent or stringent conditions and comprises at least one CpG dinucleotide sequence; and/or
a fragment that hybridizes to at least 15 nucleotides of SEQ ID NO: 33, SEQ ID NO: 34, SEQ ID NO: 35, or SEQ ID NO: 36 or a complementary sequence thereof under moderately stringent or stringent conditions and comprises at least one CpG dinucleotide sequence.

90. The method according to claim 89, wherein the primers are of sequences of SEQ ID NO: 37 and SEQ ID NO: 38, or sequences of SEQ ID NO: 40 and SEQ ID NO: 41, or sequences of SEQ ID NO: 43 and SEQ ID NO: 44, or sequences of SEQ ID NO: 46 and SEQ ID NO: 47, or sequences of SEQ ID NO: 49 and SEQ ID NO: 50, or sequences of SEQ ID NO: 52 and SEQ ID NO: 53, sequences of SEQ ID NO: 55 and SEQ ID NO: 56, or sequences of SEQ ID NO: 58 and SEQ ID NO: 59, or sequences of SEQ ID NO: 61 and SEQ ID NO: 62;
preferably, the probe is a sequence of SEQ ID NO: 39, or a sequence of SEQ ID NO: 42, or a sequence of SEQ ID NO: 45, or a sequence of SEQ ID NO: 48, or a sequence of SEQ ID NO: 51, or a sequence of SEQ ID NO: 54, or a sequence of SEQ ID NO: 57, or a sequence of SEQ ID NO: 60, or a sequence of SEQ ID NO: 63.

91. A method for detecting liver cancer, comprising:
a data acquisition step, for acquiring data on the methylation marker levels in a subject sample; and
a step of calculating the probability of suffering from liver cancer, for calculating the data obtained in the data acquisition step, thereby calculating the probability of the subject suffering from liver cancer,
wherein the methylation marker(s) is/are one, two or three of an RNF135 gene, a CHFR gene and a PAX5 gene.

92. The method according to claim 91, wherein the target sequence of the RNF135 gene is shown as SEQ ID NO: 1 or SEQ ID NO: 2 or SEQ ID NO: 3 or SEQ ID NO: 4, or comprises the sequence shown as SEQ ID NO: 1 or SEQ ID NO: 2 or SEQ ID NO: 3 or SEQ ID NO: 4; or
wherein the target sequence of the CHFR gene is shown as SEQ ID NO: 5 or SEQ ID NO: 6 or SEQ ID NO: 7 or SEQ ID NO: 8, or comprises the sequence shown as SEQ ID NO: 5 or SEQ ID NO: 6 or SEQ ID NO: 7 or SEQ ID NO: 8; or
wherein the target sequence of the PAX5 gene is shown as SEQ ID NO: 9 or SEQ ID NO: 10 or SEQ ID NO: 11 or SEQ ID NO: 12, or comprises the sequence shown as SEQ ID NO: 9 or SEQ ID NO: 10 or SEQ ID NO: 11 or SEQ ID NO: 12.

93. The method according to claim 91, wherein the data acquisition step comprises the following sub-steps:
an extraction step, for extracting genomic DNA from a sample;
a reagent treatment step, for treating the extracted genomic DNA with a bisulfite reagent to convert the 5-unmethylated cytosine base to uracil or other bases;
a detection step, for detecting the genomic DNA treated with a bisulfite reagent to obtain the methylation marker level of the sample,
preferably the genomic DNA treated with a bisulfite reagent is detected using real-time fluorescent quantitative PCR.

94. The method according to claim 93, wherein the primers used are of sequences of SEQ ID NO: 13 and SEQ ID NO: 14, or sequences of SEQ ID NO: 16 and SEQ ID NO: 17, or sequences of SEQ ID NO: 19 and SEQ ID NO: 20; or
the probe used is of the sequence of SEQ ID NO: 15, or the sequence of SEQ ID NO: 18, or the sequence of SEQ ID NO: 21.

95. The method according to claim 91, wherein in the step of calculating the probability of suffering from liver cancer, a pre-constructed formula for calculating the probability of a subject suffering from liver cancer based on data fitting of methylation marker levels is pre-stored,
preferably wherein the formula is obtained by fitting the data of the methylation level of RNF135 in an existing database.
more preferably wherein the formula is obtained by fitting the data of the methylation level of RNF135 and the methylation level of PAX5 in an existing database.

96. The method according to claim 95, wherein the formula is obtained by fitting the data of the methylation level of RNF135, the methylation level of PAX5, and the methylation level of CHFR in an existing database, preferably wherein the formula is obtained by fitting the data of the methylation level of RNF135, the methylation level of PAX5, and the methylation level of CHFR in the existing database using generalized linear regression.

97. The method according to claim 95, wherein the formula is:
$$P = {e}^{( a * M 1 + b * M 2 + c * M 3 + 81 )} / ( {e}^{( a * M 1 + b * M 2 + c * M 3 + 81 )} + 1 ) ,$$
wherein P represents the probability of the subject suffering from liver cancer, M1 represents the Ct value of RNF135 determined by real-time fluorescence quantitative PCR, M2 represents the Ct value of PAX5 determined by real-time fluorescence quantitative PCR, and M3 represents the Ct value of CHFR determined by real-time fluorescence quantitative PCR,
a is selected from any value between 0.8 and 1.2, preferably 0.9;
b is selected from any value between 0.6 and 0.9, preferably 0.7;
c is selected from any value between 0.1 and 0.5, preferably 0.3,

98. The method according to claim 91, wherein when the probability of the subject suffering from liver cancer is greater than or equal to 0.24, the subject is determined to have liver cancer,
preferably, wherein the sample comprises: cell lines, histological sections, tissue biopsies/paraffin-embedded tissues, body fluid, feces, colon effluents, urine, plasma, serum, whole blood, isolated blood cells, cells isolated from blood, or a combination thereof.


SEQUENCE LISTING (63 sequences)

SEQ ID NO: 1 - DNA, 574 bp, synthetic construct
  source 1..574: mol_type = other DNA
tggtttcccagggcaagaggccgccactgaaggtcagctctgtctttccagctgggcactggaggctctaagtctgttttcagcaggatcggaggaaggagacggggtggcgccaaggaaggaggagaaaaggcggccgagaaaaggaggagggcaaggggaagaggaagggcgagggaggagcctgaggagactcgcccggctcaaccccgacgtccgcgccccggccgcctgttggccatggcgggcctgggcctgggctccgccgttcccgtgtggctggccgaggacgacctcggctgcatcatctgccaggggctgctggactggcccgccacgctgccctgcggccacagcttctgccgccactgcctggaggccctgtggggcgcccgcgacgcccgccgctgggcctgccccacttgccgccagggcgccgcgcagcagccgcacctgcggaagaacacgctactgcaggacctggccgacaagtaccgccgcgccgcacgcgagatacaggcgggctccgaccctgcccactgcccctgcccgggctccagttccctctccagcg

SEQ ID NO: 2 - DNA, 574 bp, synthetic construct
  source 1..574: mol_type = other DNA
tggttttttagggtaagaggtcgttattgaaggttagttttgtttttttagttgggtattggaggttttaagtttgtttttagtaggatcggaggaaggagacggggtggcgttaaggaaggaggagaaaaggcggtcgagaaaaggaggagggtaaggggaagaggaagggcgagggaggagtttgaggagattcgttcggtttaatttcgacgttcgcgtttcggtcgtttgttggttatggcgggtttgggtttgggtttcgtcgttttcgtgtggttggtcgaggacgatttcggttgtattatttgttaggggttgttggattggttcgttacgttgttttgcggttatagtttttgtcgttattgtttggaggttttgtggggcgttcgcgacgttcgtcgttgggtttgttttatttgtcgttagggcgtcgcgtagtagtcgtatttgcggaagaatacgttattgtaggatttggtcgataagtatcgtcgcgtcgtacgcgagatataggcgggtttcgattttgtttattgtttttgttcgggttttagttttttttttagcg

SEQ ID NO: 3 - DNA, 574 bp, synthetic construct
  source 1..574: mol_type = other DNA
cgctggagagggaactggagcccgggcaggggcagtgggcagggtcggagcccgcctgtatctcgcgtgcggcgcggcggtacttgtcggccaggtcctgcagtagcgtgttcttccgcaggtgcggctgctgcgcggcgccctggcggcaagtggggcaggcccagcggcgggcgtcgcgggcgccccacagggcctccaggcagtggcggcagaagctgtggccgcagggcagcgtggcgggccagtccagcagcccctggcagatgatgcagccgaggtcgtcctcggccagccacacgggaacggcggagcccaggcccaggcccgccatggccaacaggcggccggggcgcggacgtcggggttgagccgggcgagtctcctcaggctcctccctcgcccttcctcttccccttgccctcctccttttctcggccgccttttctcctccttccttggcgccaccccgtctccttcctccgatcctgctgaaaacagacttagagcctccagtgcccagctggaaagacagagctgaccttcagtggcggcctcttgccctgggaaacca

SEQ ID NO: 4 - DNA, 574 bp, synthetic construct
  source 1..574: mol_type = other DNA
cgttggagagggaattggagttcgggtaggggtagtgggtagggtcggagttcgtttgtatttcgcgtgcggcgcggcggtatttgtcggttaggttttgtagtagcgtgttttttcgtaggtgcggttgttgcgcggcgttttggcggtaagtggggtaggtttagcggcgggcgtcgcgggcgttttatagggtttttaggtagtggcggtagaagttgtggtcgtagggtagcgtggcgggttagtttagtagtttttggtagatgatgtagtcgaggtcgttttcggttagttatacgggaacggcggagtttaggtttaggttcgttatggttaataggcggtcggggcgcggacgtcggggttgagtcgggcgagtttttttaggtttttttttcgtttttttttttttttttgtttttttttttttttcggtcgtttttttttttttttttttggcgttatttcgtttttttttttcgattttgttgaaaatagatttagagtttttagtgtttagttggaaagatagagttgatttttagtggcggttttttgttttgggaaatta

SEQ ID NO: 5 - DNA, 466 bp, synthetic construct
  source 1..466: mol_type = other DNA
agaggggcgggtcggggcggagagccgcgcccaaaggcaatgggagccgcacgctgctaggcaacatgctgtctcccggcaacgtagaggcggggaattcgccaacgtgtctgaaacagacaacaattttagccaacggctgcgtgggccttgggcgtcctggccaatgggaatagtgaggggcctgtcactaagaaacctgtccctgagcaacgcataggaggggtggaacccagcagcgctgactgagccgccgcgaagtgccggaacgtattacctgggagccaaatttcagagcgtcgtttgtcttcccaagcccaccagctttcagcgttctccagcctcgccacacccccgcctcgggccgtccccgcccagacgcttgtccccgcgagaagctgagcgcactgaagtgagcccggcggctgccgccggaggaggagcccggctctggacacggccgtca

SEQ ID NO: 6 - DNA, 466 bp, synthetic construct
  source 1..466: mol_type = other DNA
agaggggcgggtcggggcggagagtcgcgtttaaaggtaatgggagtcgtacgttgttaggtaatatgttgtttttcggtaacgtagaggcggggaattcgttaacgtgtttgaaatagataataattttagttaacggttgcgtgggttttgggcgttttggttaatgggaatagtgaggggtttgttattaagaaatttgtttttgagtaacgtataggaggggtggaatttagtagcgttgattgagtcgtcgcgaagtgtcggaacgtattatttgggagttaaattttagagcgtcgtttgtttttttaagtttattagtttttagcgttttttagtttcgttatattttcgtttcgggtcgttttcgtttagacgtttgttttcgcgagaagttgagcgtattgaagtgagttcggcggttgtcgtcggaggaggagttcggttttggatacggtcgtta

SEQ ID NO: 7 - DNA, 466 bp, synthetic construct
  source 1..466: mol_type = other DNA
tgacggccgtgtccagagccgggctcctcctccggcggcagccgccgggctcacttcagtgcgctcagcttctcgcggggacaagcgtctgggcggggacggcccgaggcgggggtgtggcgaggctggagaacgctgaaagctggtgggcttgggaagacaaacgacgctctgaaatttggctcccaggtaatacgttccggcacttcgcggcggctcagtcagcgctgctgggttccacccctcctatgcgttgctcagggacaggtttcttagtgacaggcccctcactattcccattggccaggacgcccaaggcccacgcagccgttggctaaaattgttgtctgtttcagacacgttggcgaattccccgcctctacgttgccgggagacagcatgttgcctagcagcgtgcggctcccattgcctttgggcgcggctctccgccccgacccgcccctct

SEQ ID NO: 8 - DNA, 466 bp, synthetic construct
  source 1..466: mol_type = other DNA
tgacggtcgtgtttagagtcgggtttttttttcggcggtagtcgtcgggtttattttagtgcgtttagtttttcgcggggataagcgtttgggcggggacggttcgaggcgggggtgtggcgaggttggagaacgttgaaagttggtgggtttgggaagataaacgacgttttgaaatttggtttttaggtaatacgtttcggtatttcgcggcggtttagttagcgttgttgggttttattttttttatgcgttgtttagggataggttttttagtgataggttttttattatttttattggttaggacgtttaaggtttacgtagtcgttggttaaaattgttgtttgttttagatacgttggcgaatttttcgtttttacgttgtcgggagatagtatgttgtttagtagcgtgcggtttttattgtttttgggcgcggtttttcgtttcgattcgttttttt

SEQ ID NO: 9 - DNA, 418 bp, synthetic construct
  source 1..418: mol_type = other DNA
taattcaagccttccgctcccccgccgagctggggtagctgatcactgagctgaaactaaacgttttaggtggaaaaaaagcgtccgaaggcaccgtgaaatgattaaggaactaaagagcttctcgccatgtgagatcatgtcctgttctcgccaacatcacaagatgtccccagacacgccgcgcccccagcgcgccgccccacactgccggcccggagcgaggaaagggtaggcgctgcgcggccgggcctgctcagcgcgccagacgtggcggacccggcccggccggagtagagcgggaagccgggagagcagcagtgctgctgccgcgccgccccagacttttataggggttggggggagggaaggaaggcttcagcctgcgccgggcgctagccagcgcacctacgggaag

SEQ ID NO: 10 - DNA, 418 bp, synthetic construct
  source 1..418: mol_type = other DNA
taatttaagtttttcgttttttcgtcgagttggggtagttgattattgagttgaaattaaacgttttaggtggaaaaaaagcgttcgaaggtatcgtgaaatgattaaggaattaaagagtttttcgttatgtgagattatgttttgttttcgttaatattataagatgtttttagatacgtcgcgtttttagcgcgtcgttttatattgtcggttcggagcgaggaaagggtaggcgttgcgcggtcgggtttgtttagcgcgttagacgtggcggattcggttcggtcggagtagagcgggaagtcgggagagtagtagtgttgttgtcgcgtcgttttagatttttataggggttggggggagggaaggaaggttttagtttgcgtcgggcgttagttagcgtatttacgggaag

SEQ ID NO: 11 - DNA, 418 bp, synthetic construct
  source 1..418: mol_type = other DNA
cttcccgtaggtgcgctggctagcgcccggcgcaggctgaagccttccttccctccccccaacccctataaaagtctggggcggcgcggcagcagcactgctgctctcccggcttcccgctctactccggccgggccgggtccgccacgtctggcgcgctgagcaggcccggccgcgcagcgcctaccctttcctcgctccgggccggcagtgtggggcggcgcgctgggggcgcggcgtgtctggggacatcttgtgatgttggcgagaacaggacatgatctcacatggcgagaagctctttagttccttaatcatttcacggtgccttcggacgctttttttccacctaaaacgtttagtttcagctcagtgatcagctaccccagctcggcgggggagcggaaggcttgaatta

SEQ ID NO: 12 - DNA, 418 bp, synthetic construct
  source 1..418: mol_type = other DNA
tttttcgtaggtgcgttggttagcgttcggcgtaggttgaagttttttttttttttttttaatttttataaaagtttggggcggcgcggtagtagtattgttgttttttcggtttttcgttttatttcggtcgggtcgggttcgttacgtttggcgcgttgagtaggttcggtcgcgtagcgtttatttttttttcgtttcgggtcggtagtgtggggcggcgcgttgggggcgcggcgtgtttggggatattttgtgatgttggcgagaataggatatgattttatatggcgagaagttttttagttttttaattattttacggtgttttcggacgttttttttttatttaaaacgtttagttttagtttagtgattagttattttagttcggcgggggagcggaaggtttgaatta

SEQ ID NO: 13 - DNA, 467 bp, synthetic construct
  source 1..467: mol_type = other DNA
cgccccgccccgccaggttctgttgggggcgaggcccgcgcaagccccgcctcttccccggcaccaggggcgggcccaggtgcgcccagggccggggagcggccgcgcaggtgcctgccctttgcgcctgcgcccagctcgccctgcctagccaggtgcgccccgccccctgcctgcccggccaccttcgggagccgcttccaataggcgttcgccattggctctggcgacctccgcgcgttgggaggtgtagcgcggctctgaacgcgctgagggccgttgagtgtcgcaggcggcgagggcgcgagtgaggagcagacccaggcatcgcgcgccgagaaggccgggcgtccccacactgaaggtccggaaaggcgacttccgggggctttggcacctggcggaccctcccggagcgtcggcacctgaacgcgaggcgctccattgcgcgtgcgcgttgaggggct

SEQ ID NO: 14 - DNA, 467 bp, synthetic construct
  source 1..467: mol_type = other DNA
cgtttcgtttcgttaggttttgttgggggcgaggttcgcgtaagtttcgtttttttttcggtattaggggcgggtttaggtgcgtttagggtcggggagcggtcgcgtaggtgtttgttttttgcgtttgcgtttagttcgttttgtttagttaggtgcgtttcgttttttgtttgttcggttattttcgggagtcgtttttaataggcgttcgttattggttttggcgattttcgcgcgttgggaggtgtagcgcggttttgaacgcgttgagggtcgttgagtgtcgtaggcggcgagggcgcgagtgaggagtagatttaggtatcgcgcgtcgagaaggtcgggcgtttttatattgaaggttcggaaaggcgattttcgggggttttggtatttggcggattttttcggagcgtcggtatttgaacgcgaggcgttttattgcgcgtgcgcgttgaggggtt

SEQ ID NO: 15 - DNA, 467 bp, synthetic construct
  source 1..467: mol_type = other DNA
agcccctcaacgcgcacgcgcaatggagcgcctcgcgttcaggtgccgacgctccgggagggtccgccaggtgccaaagcccccggaagtcgcctttccggaccttcagtgtggggacgcccggccttctcggcgcgcgatgcctgggtctgctcctcactcgcgccctcgccgcctgcgacactcaacggccctcagcgcgttcagagccgcgctacacctcccaacgcgcggaggtcgccagagccaatggcgaacgcctattggaagcggctcccgaaggtggccgggcaggcagggggcggggcgcacctggctaggcagggcgagctgggcgcaggcgcaaagggcaggcacctgcgcggccgctccccggccctgggcgcacctgggcccgcccctggtgccggggaagaggcggggcttgcgcgggcctcgcccccaacagaacctggcggggcggggcg

SEQ ID NO: 16 - DNA, 467 bp, synthetic construct
  source 1..467: mol_type = other DNA
agttttttaacgcgtacgcgtaatggagcgtttcgcgtttaggtgtcgacgtttcgggagggttcgttaggtgttaaagttttcggaagtcgttttttcggatttttagtgtggggacgttcggttttttcggcgcgcgatgtttgggtttgttttttattcgcgttttcgtcgtttgcgatatttaacggtttttagcgcgtttagagtcgcgttatattttttaacgcgcggaggtcgttagagttaatggcgaacgtttattggaagcggttttcgaaggtggtcgggtaggtagggggcggggcgtatttggttaggtagggcgagttgggcgtaggcgtaaagggtaggtatttgcgcggtcgtttttcggttttgggcgtatttgggttcgtttttggtgtcggggaagaggcggggtttgcgcgggtttcgtttttaatagaatttggcggggcggggcg

SEQ ID NO: 17 - DNA, 421 bp, synthetic construct
  source 1..421: mol_type = other DNA
aagggaagtaaacggccccaacgcaagcctgactgcgagacgtgcccaagggaggtaggtcgaatgaagagggttgtgtgagttttgcgcgggcaggcgggataacggaggagggaggcccgcggccgaggctcgggcgggcggggggcagggaggggcggggttcgccggcgcgcactcccaggcaggccccgccccctcggccggctgtgcgcgctgattggcccctgccggcctcgcgctccctcgctccgggttggcgggagaccttagagcgggtaccgctgctggctagcgaccgacgagcaaccgtctgaggccaggagcgctgcgacggagccttgaccgccgttgcccggccctctcccgcgcagccccgggcttccgcaggtacctgtgctcgcccccgggaaggggcctc

SEQ ID NO: 18 - DNA, 421 bp, synthetic construct
  source 1..421: mol_type = other DNA
aagggaagtaaacggttttaacgtaagtttgattgcgagacgtgtttaagggaggtaggtcgaatgaagagggttgtgtgagttttgcgcgggtaggcgggataacggaggagggaggttcgcggtcgaggttcgggcgggcggggggtagggaggggcggggttcgtcggcgcgtatttttaggtaggtttcgttttttcggtcggttgtgcgcgttgattggtttttgtcggtttcgcgttttttcgtttcgggttggcgggagattttagagcgggtatcgttgttggttagcgatcgacgagtaatcgtttgaggttaggagcgttgcgacggagttttgatcgtcgttgttcggttttttttcgcgtagtttcgggttttcgtaggtatttgtgttcgttttcgggaaggggtttt

SEQ ID NO: 19 - DNA, 421 bp, synthetic construct
  source 1..421: mol_type = other DNA
gaggccccttcccgggggcgagcacaggtacctgcggaagcccggggctgcgcgggagagggccgggcaacggcggtcaaggctccgtcgcagcgctcctggcctcagacggttgctcgtcggtcgctagccagcagcggtacccgctctaaggtctcccgccaacccggagcgagggagcgcgaggccggcaggggccaatcagcgcgcacagccggccgagggggcggggcctgcctgggagtgcgcgccggcgaaccccgcccctccctgccccccgcccgcccgagcctcggccgcgggcctccctcctccgttatcccgcctgcccgcgcaaaactcacacaaccctcttcattcgacctacctcccttgggcacgtctcgcagtcaggcttgcgttggggccgtttacttccctt

SEQ ID NO: 20 - DNA, 421 bp, synthetic construct
  source 1..421: mol_type = other DNA
gaggttttttttcgggggcgagtataggtatttgcggaagttcggggttgcgcgggagagggtcgggtaacggcggttaaggtttcgtcgtagcgtttttggttttagacggttgttcgtcggtcgttagttagtagcggtattcgttttaaggtttttcgttaattcggagcgagggagcgcgaggtcggtaggggttaattagcgcgtatagtcggtcgagggggcggggtttgtttgggagtgcgcgtcggcgaatttcgtttttttttgtttttcgttcgttcgagtttcggtcgcgggtttttttttttcgttatttcgtttgttcgcgtaaaatttatataattttttttattcgatttattttttttgggtacgtttcgtagttaggtttgcgttggggtcgtttatttttttt

SEQ ID NO: 21 - DNA, 610 bp, synthetic construct
  source 1..610: mol_type = other DNA
cagctgtggcagaacagtcagggcctgcctgacctcttgagttttattgtctcctctcaacttgggcttaaagggactttattaaccatgactaataccagaggatccaaagctgcacccacttgttaattttgaggaatttttaaatgcgggatttaaaaaagatttaatttcacgcataggtatgaacagcccctcatcatcccaggtagaggatgaacaactcatcatcccgggtagaggcaggtaagtgtgtccagcgtccctcagggaatgctcttctgatgccaaaccctctgcttccgcggggtcacttctcgggtggttttgtttctcctctgcttccacagttgcaaaccctcgaagtgccacgacccctggggaaactcgatcttaagtcccatggccaaacgctccagcccgcggggctctcaggacaccacagcgatcggatttcgcgtggccgccaggctccccctactcctgtcccctcccgccagagaacccctggagccaatcagagcgtcgcggagcccggggttgtagagtcccgagcccgggacccgcccctctttgttgcggtggccaatagacgcattcggaacatcgg

SEQ ID NO: 22 - DNA, 610 bp, synthetic construct
  source 1..610: mol_type = other DNA
tagttgtggtagaatagttagggtttgtttgattttttgagttttattgtttttttttaatttgggtttaaagggattttattaattatgattaatattagaggatttaaagttgtatttatttgttaattttgaggaatttttaaatgcgggatttaaaaaagatttaattttacgtataggtatgaatagttttttattattttaggtagaggatgaataatttattatttcgggtagaggtaggtaagtgtgtttagcgttttttagggaatgtttttttgatgttaaattttttgttttcgcggggttatttttcgggtggttttgttttttttttgtttttatagttgtaaattttcgaagtgttacgatttttggggaaattcgattttaagttttatggttaaacgttttagttcgcggggtttttaggatattatagcgatcggatttcgcgtggtcgttaggttttttttatttttgttttttttcgttagagaatttttggagttaattagagcgtcgcggagttcggggttgtagagtttcgagttcgggattcgtttttttttgttgcggtggttaatagacgtattcggaatatcgg

SEQ ID NO: 23 - DNA, 610 bp, synthetic construct
  source 1..610: mol_type = other DNA
ccgatgttccgaatgcgtctattggccaccgcaacaaagaggggcgggtcccgggctcgggactctacaaccccgggctccgcgacgctctgattggctccaggggttctctggcgggaggggacaggagtagggggagcctggcggccacgcgaaatccgatcgctgtggtgtcctgagagccccgcgggctggagcgtttggccatgggacttaagatcgagtttccccaggggtcgtggcacttcgagggtttgcaactgtggaagcagaggagaaacaaaaccacccgagaagtgaccccgcggaagcagagggtttggcatcagaagagcattccctgagggacgctggacacacttacctgcctctacccgggatgatgagttgttcatcctctacctgggatgatgaggggctgttcatacctatgcgtgaaattaaatcttttttaaatcccgcatttaaaaattcctcaaaattaacaagtgggtgcagctttggatcctctggtattagtcatggttaataaagtccctttaagcccaagttgagaggagacaataaaactcaagaggtcaggcaggccctgactgttctgccacagctg

SEQ ID NO: 24 - DNA, 610 bp, synthetic construct
  source 1..610: mol_type = other DNA
tcgatgtttcgaatgcgtttattggttatcgtaataaagaggggcgggtttcgggttcgggattttataatttcgggtttcgcgacgttttgattggttttaggggttttttggcgggaggggataggagtagggggagtttggcggttacgcgaaattcgatcgttgtggtgttttgagagtttcgcgggttggagcgtttggttatgggatttaagatcgagtttttttaggggtcgtggtatttcgagggtttgtaattgtggaagtagaggagaaataaaattattcgagaagtgatttcgcggaagtagagggtttggtattagaagagtattttttgagggacgttggatatatttatttgtttttattcgggatgatgagttgtttattttttatttgggatgatgaggggttgtttatatttatgcgtgaaattaaattttttttaaatttcgtatttaaaaattttttaaaattaataagtgggtgtagttttggattttttggtattagttatggttaataaagtttttttaagtttaagttgagaggagataataaaatttaagaggttaggtaggttttgattgttttgttatagttg

SEQ ID NO: 25 - DNA, 355 bp, synthetic construct
  source 1..355: mol_type = other DNA
caagtatttttctgcaaattagaacggggcgttcccctcccccatcaccccaattccagaggacgtcaatcacagtggacgcagcaccgcgagcggaaatgcggagagcatcggggctccgcatgtggggcacaatgaaacaagagcagccccggccagaggtccagtaaagcggaaagacggtcccgcccgcacacccctccggaaaactgaagcgtctcctctgacccccaaggtcagaaagatttattacttccttcgagagggcgcagccacagacccactagagagggcgggcagggtgtcggaagccagagaacggcgctccgcgtcctcccaccgctccagggcgcag

SEQ ID NO: 26 - DNA, 355 bp, synthetic construct
  source 1..355: mol_type = other DNA
taagtatttttttgtaaattagaacggggcgttttttttttttattattttaattttagaggacgttaattatagtggacgtagtatcgcgagcggaaatgcggagagtatcggggtttcgtatgtggggtataatgaaataagagtagtttcggttagaggtttagtaaagcggaaagacggtttcgttcgtatattttttcggaaaattgaagcgttttttttgatttttaaggttagaaagatttattatttttttcgagagggcgtagttatagatttattagagagggcgggtagggtgtcggaagttagagaacggcgtttcgcgtttttttatcgttttagggcgtag

SEQ ID NO: 27 - DNA, 355 bp, synthetic construct
  source 1..355: mol_type = other DNA
ctgcgccctggagcggtgggaggacgcggagcgccgttctctggcttccgacaccctgcccgccctctctagtgggtctgtggctgcgccctctcgaaggaagtaataaatctttctgaccttgggggtcagaggagacgcttcagttttccggaggggtgtgcgggcgggaccgtctttccgctttactggacctctggccggggctgctcttgtttcattgtgccccacatgcggagccccgatgctctccgcatttccgctcgcggtgctgcgtccactgtgattgacgtcctctggaattggggtgatgggggaggggaacgccccgttctaatttgcagaaaaatacttg

SEQ ID NO: 28 - DNA, 355 bp, synthetic construct
  source 1..355: mol_type = other DNA
ttgcgttttggagcggtgggaggacgcggagcgtcgttttttggttttcgatattttgttcgttttttttagtgggtttgtggttgcgttttttcgaaggaagtaataaatttttttgattttgggggttagaggagacgttttagtttttcggaggggtgtgcgggcgggatcgttttttcgttttattggatttttggtcggggttgtttttgttttattgtgttttatatgcggagtttcgatgtttttcgtattttcgttcgcggtgttgcgtttattgtgattgacgttttttggaattggggtgatgggggaggggaacgtttcgttttaatttgtagaaaaatatttg

SEQ ID NO: 29 - DNA, 306 bp, synthetic construct
  source 1..306: mol_type = other DNA
cggactcgggctttggcgcggcctttgccccggtttttggcgcgggaggactttcgaccccgacttcggccgctcatggtggcggcggaggcagcttcaaagacacgctgtgaccctgcggctcctgacgccagctctcggtcgggaccgagcgggtctctccacggcaaccgccgacgtcacgaacgtacaactgtaccgtcgcgagaggacgtgatgcgcccggtgattggcgccgccgctgcggctgcgcaggagacgacccccgcgggcgctcccacccccatctcgcgcggactcgcttta

SEQ ID NO: 30 - DNA, 306 bp, synthetic construct
  source 1..306: mol_type = other DNA
cggattcgggttttggcgcggtttttgtttcggtttttggcgcgggaggattttcgatttcgatttcggtcgtttatggtggcggcggaggtagttttaaagatacgttgtgattttgcggtttttgacgttagttttcggtcgggatcgagcgggtttttttacggtaatcgtcgacgttacgaacgtataattgtatcgtcgcgagaggacgtgatgcgttcggtgattggcgtcgtcgttgcggttgcgtaggagacgattttcgcgggcgtttttatttttatttcgcgcggattcgtttta

SEQ ID NO: 31 - DNA, 306 bp, synthetic construct
  source 1..306: mol_type = other DNA
taaagcgagtccgcgcgagatgggggtgggagcgcccgcgggggtcgtctcctgcgcagccgcagcggcggcgccaatcaccgggcgcatcacgtcctctcgcgacggtacagttgtacgttcgtgacgtcggcggttgccgtggagagacccgctcggtcccgaccgagagctggcgtcaggagccgcagggtcacagcgtgtctttgaagctgcctccgccgccaccatgagcggccgaagtcggggtcgaaagtcctcccgcgccaaaaaccggggcaaaggccgcgccaaagcccgagtccg

SEQ ID NO: 32 - DNA, 306 bp, synthetic construct
  source 1..306: mol_type = other DNA
taaagcgagttcgcgcgagatgggggtgggagcgttcgcgggggtcgttttttgcgtagtcgtagcggcggcgttaattatcgggcgtattacgttttttcgcgacggtatagttgtacgttcgtgacgtcggcggttgtcgtggagagattcgttcggtttcgatcgagagttggcgttaggagtcgtagggttatagcgtgtttttgaagttgttttcgtcgttattatgagcggtcgaagtcggggtcgaaagttttttcgcgttaaaaatcggggtaaaggtcgcgttaaagttcgagttcg

SEQ ID NO: 33 - DNA, 400 bp, synthetic construct
  source 1..400: mol_type = other DNA
ccgagggccgggggcgcctggagagaaatccagctccggctctgagcgtctccagtcaggcgaggcggataaatccttcgcaaaaccctcttggaaattgccgccgcttcctgagccatcagtcccagcgggtacgttatcgagtagcacaaacagttggatttttccctcaagaaccgagtctggacgcggagatggagccaagtgtggctgcattttcggacccggaaatccgttgggcactgaaggacttttcgaaccctgtagcgctgttgcttcgcggtccatcgtcgccgctgcagacggatgcgctccccggcggctctacgccctccagtcccggccaggcctctgggctgggagccgagccgtctcgggccctccggcgccgcgttttcta

SEQ ID NO: 34 - DNA, 400 bp, synthetic construct
  source 1..400: mol_type = other DNA
tcgagggtcgggggcgtttggagagaaatttagtttcggttttgagcgtttttagttaggcgaggcggataaatttttcgtaaaatttttttggaaattgtcgtcgttttttgagttattagttttagcgggtacgttatcgagtagtataaatagttggattttttttttaagaatcgagtttggacgcggagatggagttaagtgtggttgtattttcggattcggaaattcgttgggtattgaaggatttttcgaattttgtagcgttgttgtttcgcggtttatcgtcgtcgttgtagacggatgcgtttttcggcggttttacgttttttagtttcggttaggtttttgggttgggagtcgagtcgtttcgggtttttcggcgtcgcgtttttta

SEQ ID NO: 35 - DNA, 400 bp, synthetic construct
  source 1..400: mol_type = other DNA
tagaaaacgcggcgccggagggcccgagacggctcggctcccagcccagaggcctggccgggactggagggcgtagagccgccggggagcgcatccgtctgcagcggcgacgatggaccgcgaagcaacagcgctacagggttcgaaaagtccttcagtgcccaacggatttccgggtccgaaaatgcagccacacttggctccatctccgcgtccagactcggttcttgagggaaaaatccaactgtttgtgctactcgataacgtacccgctgggactgatggctcaggaagcggcggcaatttccaagagggttttgcgaaggatttatccgcctcgcctgactggagacgctcagagccggagctggatttctctccaggcgcccccggccctcgg

SEQ ID NO: 36 - DNA, 400 bp, synthetic construct
  source 1..400: mol_type = other DNA
tagaaaacgcggcgtcggagggttcgagacggttcggtttttagtttagaggtttggtcgggattggagggcgtagagtcgtcggggagcgtattcgtttgtagcggcgacgatggatcgcgaagtaatagcgttatagggttcgaaaagttttttagtgtttaacggattttcgggttcgaaaatgtagttatatttggttttattttcgcgtttagattcggtttttgagggaaaaatttaattgtttgtgttattcgataacgtattcgttgggattgatggtttaggaagcggcggtaatttttaagagggttttgcgaaggatttattcgtttcgtttgattggagacgtttagagtcggagttggatttttttttaggcgttttcggttttcgg

SEQ ID NO: 37 - DNA, 18 bp, synthetic construct
  source 1..18: mol_type = other DNA
gggtggaatttagtagcg

SEQ ID NO: 38 - DNA, 28 bp, synthetic construct
  source 1..28: mol_type = other DNA
ctctaaaatttaactcccaaataatacg

SEQ ID NO: 39 - DNA, 22 bp, synthetic construct
  source 1..22: mol_type = other DNA
ttgagtcgtcgcgaagtgtcgg

SEQ ID NO: 40 - DNA, 20 bp, synthetic construct
  source 1..20: mol_type = other DNA
tattggttttggcgattttc

SEQ ID NO: 41 - DNA, 18 bp, synthetic construct
  source 1..18: mol_type = other DNA
actcaacgaccctcaacg

SEQ ID NO: 42 - DNA, 21 bp, synthetic construct
  source 1..21: mol_type = other DNA
aggtgtagcgcggttttgaac

SEQ ID NO: 43 - DNA, 21 bp, synthetic construct
  source 1..21: mol_type = other DNA
taggggttgttggattggttc

SEQ ID NO: 44 - DNA, 23 bp, synthetic construct
  source 1..23: mol_type = other DNA
cacaaaacctccaaacaataacg

SEQ ID NO: 45 - DNA, 29 bp, synthetic construct
  source 1..29: mol_type = other DNA
tacgttgttttgcggttatagtttttgtc

SEQ ID NO: 46 - DNA, 23 bp, synthetic construct
  source 1..23: mol_type = other DNA
taagatgtttttagatacgtcgc

SEQ ID NO: 47 - DNA, 16 bp, synthetic construct
  source 1..16: mol_type = other DNA
gcctaccctttcctcg

SEQ ID NO: 48 - DNA, 23 bp, synthetic construct
  source 1..23: mol_type = other DNA
cgacaatataaaacgacgcgcta

SEQ ID NO: 49 - DNA, 25 bp, synthetic construct
  source 1..25: mol_type = other DNA
ttttaagtttattagtttttagcgt

SEQ ID NO: 50 - DNA, 18 bp, synthetic construct
  source 1..18: mol_type = other DNA
ttctcgcgaaaacaaacg

SEQ ID NO: 51 - DNA, 22 bp, synthetic construct
  source 1..22: mol_type = other DNA
aaacgaaaacgacccgaaacga

SEQ ID NO: 52 - DNA, 20 bp, synthetic construct
  source 1..20: mol_type = other DNA
agatacgtcgcgtttttagc

SEQ ID NO: 53 - DNA, 17 bp, synthetic construct
  source 1..17: mol_type = other DNA
cgcctaccctttcctcg

SEQ ID NO: 54 - DNA, 25 bp, synthetic construct
  source 1..25: mol_type = other DNA
tcgttttatattgtcggttcggagc

SEQ ID NO: 55 - DNA, 24 bp, synthetic construct
  source 1..24: mol_type = other DNA
gcggggtttttaggatattatagc

SEQ ID NO: 56 - DNA, 28 bp, synthetic construct
  source 1..28: mol_type = other DNA
aaaaaacaaaaataaaaaaaacctaacg

SEQ ID NO: 57 - DNA, 20 bp, synthetic construct
  source 1..20: mol_type = other DNA
gcgatcggatttcgcgtggt

SEQ ID NO: 58 - DNA, 17 bp, synthetic construct
  source 1..17: mol_type = other DNA
cgttagttttcggtcgg

SEQ ID NO: 59 - DNA, 20 bp, synthetic construct
  source 1..20: mol_type = other DNA
cgacgatacaattatacgtt

SEQ ID NO: 60 - DNA, 18 bp, synthetic construct
  source 1..18: mol_type = other DNA
acggtaatcgtcgacgtt

SEQ ID NO: 61 - DNA, 19 bp, synthetic construct
  source 1..19: mol_type = other DNA
ttcgaattttgtagcgttg

SEQ ID NO: 62 - DNA, 16 bp, synthetic construct
  source 1..16: mol_type = other DNA
gccgaaaaacgcatcc

SEQ ID NO: 63 - DNA, 21 bp, synthetic construct
  source 1..21: mol_type = other DNA
ctacaacgacgacgataaacc